(19) United States
(12) Patent Application Publication
(10) Pub. No.: US 2011/0269652 A1
(43) Pub. Date: Nov. 3, 2011

(54) GEMINI SURFACTANTS

(75) Inventors: D. Gerrard Marangoni, Antigonish (CA); T. Bruce Grindley, Halifax (CA); Nusrat Jahan, Halifax (CA); Christian Petropolis, Halifax (CA); Thomas Tran, North York (CA); Nawal Paul, Halifax (CA)

(73) Assignee: ST. FRANCIS XAVIER UNIVERSITY, Antigonish, NS (CA)

(21) Appl. No.: 13/126,632

(22) PCT Filed: Oct. 29, 2009
(86) PCT No.: PCT/CA09/01549

§ 371 Date: Jul. 12, 2011

Related U.S. Application Data
(60) Provisional application No. 61/197,760, filed on Oct. 30, 2008.
(60) Provisional application No. 61/199,640, filed on Nov. 19, 2008.

(51) Int. Cl. C09K 8/60; C07C 305/08; C07C 233/02; C07C 211/09; C07C 55/02; C07C 255/14; C07D 403/06; C07C 43/04; C07C 229/26; C07D 319/06; C11D 1/72; C11D 1/66; C11D 1/62; C11D 1/90; C11D 1/04; C11D 1/29; C07C 211/63

(52) U.S. Cl. 507/240; 564/295; 558/26; 562/103; 564/160; 564/463; 562/590; 558/447; 548/524; 568/614; 560/169; 549/374; 510/506; 510/499; 510/504; 510/490; 510/500; 510/501; 510/495; 507/261; 507/246; 507/260; 507/254; 507/255

(57) ABSTRACT

The invention relates to gemini surfactants of formula IA wherein A is a core derived from an organic polyhydroxy compound; R¹ and R² are each independently a hydrophobic group; and R³ and R⁴ are each independently a surfactant head group. Such surfactants can be used as components of fluids used in the petroleum industry or used in formulating cleansing compositions or detergent compositions.

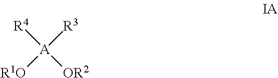

### FIELD OF THE INVENTION

The present invention relates to surface active agents derived from organic polyhydroxy compounds, more particularly to gemini surfactants derived from organic polyhydroxy compounds. The present invention further relates to the use of these surfactants as cleansing agents or in industry, including in fluid systems utilized in the petroleum industry.

### BACKGROUND OF THE INVENTION

Surfactants, or surface active agents, either dispersed in solution as monomers or as aggregates (e.g., spherical micelles), are used widely in a number of industrial and pharmaceutical processes. In addition, surfactants are used as cleansers, detergents and emulsifying agents and are found in a wide range of personal care and household products such as shampoos, laundry detergents and dishwashing detergents. Surfactants also find use in a variety of fluid and remediation technologies used in the oil-services industry. For example, surfactants are routinely used as wetting agents and emulsifiers in both water based and oil based drilling fluids, and are effective in preventing accretion, the process by which drilled cuttings and the metal tools used in the drilling process often become coated with a gummy, resinous film when wells are drilled through oil sands.

In addition, surfactants are useful in hydraulic fracturing, a process used to treat either depleted wells in later stages of production or wells in reservoirs of low permeability. In this process, the well is treated with a fluid system at a pressure high enough to fracture the formation, which creates new channels permitting the flow of oil and gas to the well bore. In many cases, the fracturing fluid is a polymeric solution of sufficient viscosity to suspend a large quantity of particulate matter (known as the proppant), the purpose of which is to prop open the fractures and maintain the flow pathways after the fluid solution is either removed to the surface or is subsequently lost to the formation. Surfactants forming worm-like micelles are especially useful as a component in fracturing fluids, because of their favourable viscoelastic properties.

Surfactants are also used in stimulation fluids, which are injected into a formation at a distance from the producing well under relatively high pressures to create a driving force to squeeze more oil from the production zone. The surfactants act to reduce the interfacial energy between the near well bore and the producing fluid and to help solubilize waxy materials that often precipitate out in the near well bore area and reduce the permeability of the zone. The selection and use of a suitable surfactant can result in vastly improved recoveries from underground reservoirs. The correlation between the ability to reduce the energy required to create new surfaces and interfaces and the ability to mobilize reservoir entrapped petroleum reserves is described by Schramm et al (Schramm, L. L.; Smith, R. G.; Stone, J. A. Colloids and Surfaces 1984, 11, 247-263).

Because of their ability to increase the bioavailability of the oil to bacteria in the remediation cycle, surfactants are also used to accelerate bioremediation, or the bacterial removal of oil from cuttings formed during the drilling of oil wells. A critical element in the application of bioremediation technologies is the tendency of surfactant solutions to lower the energy required to create new interfacial area. The ability of the surfactant to lift grease and oil from a solid matrix is directly related to its wetting ability; hence the wetting ability, and the ability of the surfactant to lower surface and interfacial tension, are both key parameters in assessing the utility of surfactants in bioremediation applications. Wetting abilities are closely related to the efficiency with which the surfactant molecules preferentially adsorb at solid surfaces and liquid interfaces.

The performance of common surfactants in various applications has been investigated (for example, see Detergency of Specialty Surfactants; Marcel Dekker: New York, 2001; Vol. 98 and Guyot, A. Adv. Colloid Interf. Sci. 2004, 108, 3-22). Common conventional surfactants generally contain a single polar or ionic hydrophilic headgroup (e.g., sulfate or carboxylate) covalently bound to a single hydrophobic linear or branched hydrocarbon or fluorocarbon chain. The polar or ionic headgroup interacts strongly with an aqueous environment and is solvated via dipole-dipole or ion-dipole interactions, while the nonpolar hydrophobic chains interact only very weakly with water, resulting in the formation of ordered water molecules in the vicinity of the nonpolar chain, termed the ‘hydrophobic effect’ (Southall, N. T.; Dill, K. A.; Haymet, A. D. J. J. Phys. Chem. B 2002, 106, 521-533). Because of this, surfactant molecules, which are amphiphilic, interacting strongly with both hydrophilic and hydrophobic phases, will tend to adsorb at an air-water interface, thus lowering the surface tension and reducing the Gibbs energy at the air-water interface.

Surfactants self-assemble at a specific concentration (the critical micelle concentration, or CMC value) into molecular aggregates, known as micelles. If the surfactant is ionic, the self-assembly process is accompanied by the adsorption of counterions at the micellar surface. Generally, ionic surfactants are not fully neutralized at the micellar surface and the self-assembled unit will possess a charge. The number of counterions adsorbed at the micellar surface per number of charged headgroups at the interface is known as the degree of counterion binding (β). β-values for ionic surfactants are typically in the range of 0.4-0.7. β-values are determined primarily by conductivity experiments (Jobe, D. J.; Reinsborough, V. C. Aust. J. Chem. 1984, 37, 303-310), ion-selective electrode experiments (Palepu, R.; Hall, D. G.; Wyn-Jones, E. J. Chem. Soc., Faraday Trans. 1990, 86, 1535-1538), and NMR counterion relaxation rates and chemical shifts (Chachaty, C. Prog. Nucl. Magn. Reson. Spectrosc. 1987, 19, 183-222).

Micelles are termed association colloids, and are generally thought to be spherical at concentrations slightly above the CMC value (Chang, N. J.; Kaler, E. W. J. Phys. Chem. 1985, 89, 2996-3000). The aggregation number (the number of surfactant molecules per micelle) of common surfactant micelles is generally in the range of about 50-100 monomers, with a radius similar to that of the length of an extended hydrocarbon chain (Gorski, N.; Kalus, J. Langmuir 2001, 17, 4211-4215). The micellar interior, being composed essentially of hydrocarbon chains, has properties closely related to a liquid hydrocarbon (Söderman, O.; Stilbs, P. Prog. Nucl. Magn. Reson. Spectrosc. 1994, 26, 445-482).

The term “gemini surfactant” has become accepted in the surfactant literature for describing dimeric surfactants (Menger, F. M.; Littau, C. A. J. Am. Chem. Soc. 1991, 113, 1451-1452; Zana, R.; Xia, J. Introduction. In Gemini Surfactants: Synthesis, Interfacial and Solution-phase Behaviour, and Applications, Zana, R., Xia, J., Eds.; Marcel Dekker: New York, 2004; pp 1-8), that is, surfactant molecules that have two hydrophilic (chiefly ionic) groups and two tails per surfactant molecule. These twin parts of the surfactants are linked by a spacer group of varying length (most commonly a methylene spacer or an oxyethylene spacer). FIG. 1 shows a block diagram of a typical gemini surfactant. The term gemini surfactant is also used to describe surfactants with more than two heads and tails.

Gemini surfactants can have significant advantages over existing single-headed, single-tailed surfactants in a variety of applications because of their advantageous properties (Menger, F. M.; Littau, C. A. J. Am. Chem. Soc. 1991, 113, 1451-1452; Menger, F. M.; Keiper, J. S. Angew. Chem. Int. Ed. 2000, 39, 1907-1920; Zana, R. Adv. Colloid Interf. Sci. 2002, 97, 205-253; Rosen, M. J. Cosmetics & Toiletries 1998, 113, 49-55). In general, gemini surfactants are more efficient at forming micelles and at adsorbing at the air-water interface than conventional surfactants, resulting in a large reduction in surface tension for a relatively small amount of added gemini surfactant.

Although gemini surfactants have existed since the 1930's, (Rosen, M. J. Chemtech 1993, 23, 30-33) and are commercially available from the Dow Chemical Company as the Dowfax® surfactants and from Air Products as the Surfynol® surfactants, the surfactants used in the production of fluids for use in oil well drilling or subsequent remediation generally consist of mixtures of single-headed, single-tailed species. Thus there is a need in the oil and gas industry for new surfactants which have the beneficial properties of gemini surfactants.

### SUMMARY OF THE INVENTION

The present invention provides novel gemini surfactants which find particular use in industry, including the petroleum industry.

In one aspect, the present invention provides a compound of formula IA

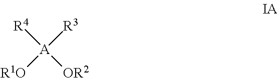

wherein A is a core derived from an organic polyhydroxy compound;

R¹ and R² are each independently a hydrophobic group; and

R³ and R⁴ are each independently a surfactant headgroup.

Another aspect of the present invention provides the use of a compound of formula IA as defined herein as a surfactant.

Another aspect of the present invention provides a fluid for use in the production or recovery of petroleum from petroleum-bearing formations, the fluid comprising a compound of formula IA as defined herein.

According to another aspect of the present invention, there is provided a method of using a fluid comprising a compound of formula IA as defined herein in the production or recovery of petroleum from petroleum-bearing formations.

### BRIEF DESCRIPTION OF THE DRAWINGS

Specific embodiments of the present invention are now described in greater detail and can be better understood by the skilled person when read in conjunction with the drawings in which:

FIG. 1 is a block diagram of a typical gemini surfactant;

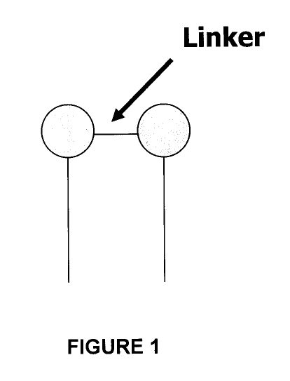

FIG. 2 is a plot of surface tension (mN·m⁻¹) versus log₁₀ of the total surfactant concentration (molar) for Compounds 5a-5d (Examples 5A-5D); and

FIG. 3 is a plot of surface tension (mN·m⁻¹) versus log₁₀ of the total surfactant concentration (molar) for compounds 22c-22f (Examples 22C-22F).

### DEFINITIONS

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The terms “alkyl” or “(C₁₋ₙ)alkyl” as used herein and unless specified otherwise, wherein n is an integer, either alone or in combination with another radical, are intended to mean an acyclic or cyclic, straight or branched chain, saturated or unsaturated alkyl radical containing from 1 to n carbon atoms. “Alkyl” includes, but is not limited to, methyl, ethyl, propyl (n-propyl), butyl (n-butyl), 1-methylethyl (iso-propyl), 1-methylpropyl (sec-butyl), 2-methylpropyl (iso-butyl), 1,1-dimethylethyl (tert-butyl), pentyl (n-pentyl), hexyl (n-hexyl), octyl (n-octyl), decyl (n-decyl), dodecyl (n-dodecyl), and tetradecyl (n-tetradecyl). The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group. Unsaturated alkyl groups include alkenyl and alkynyl groups. Cyclic alkyl groups include cycloalkyl groups.

The terms “alkenyl” or “(C₂₋ₙ)alkenyl”, as used herein and unless specified otherwise, wherein n is an integer, either alone or in combination with another radical, are intended to mean an unsaturated, acyclic straight or branched chain radical containing two to n carbon atoms, at least two of which are bonded to each other by a double bond. Examples of such radicals include, but are not limited to, ethenyl (vinyl), 1-propenyl, 2-propenyl, and 1-butenyl. Unless specified otherwise, the term “(C₂₋ₙ)alkenyl” is understood to encompass individual stereoisomers where possible, including but not limited to (E) and (Z) isomers, and mixtures thereof. When a (C₂₋ₙ)alkenyl group is substituted, it is understood to be substituted on any carbon atom thereof which would otherwise bear a hydrogen atom, unless specified otherwise, such that the substitution would give rise to a chemically stable compound.

The terms “alkynyl” or “(C₂₋ₙ)alkynyl”, as used herein and unless specified otherwise, wherein n is an integer, either alone or in combination with another radical, are intended to mean an unsaturated, acyclic straight or branched chain radical containing two to n carbon atoms, at least two of which are bonded to each other by a triple bond. Examples of such radicals include, but are not limited to, ethynyl, 1-propynyl, 2-propynyl, and 1-butynyl. When a (C₂₋ₙ)alkynyl group is substituted, it is understood to be substituted on any carbon atom thereof which would otherwise bear a hydrogen atom, unless specified otherwise, such that the substitution would give rise to a chemically stable compound.

The terms “cycloalkyl” or “(C₃₋ₘ)cycloalkyl” as used herein and unless specified otherwise, wherein m is an integer, either alone or in combination with another radical, are intended to mean a saturated or unsaturated cycloalkyl substituent containing from 3 to m carbon atoms and includes, but is not limited to, cyclopropyl, cyclobutyl, cyclopentyl, cyclopentenyl, cyclohexyl, cyclohexenyl and cycloheptyl.

The terms “alkoxy” or “(C₁₋ₙ)alkoxy” as used herein and unless specified otherwise, wherein n is an integer, either alone or in combination with another radical, are intended to mean an oxygen atom further bonded to a saturated alkyl group containing 1 to n carbon atoms as defined above. “Alkoxy” includes, but is not limited to, methoxy (—OCH₃), ethoxy (—OCH₂CH₃), propoxy (—OCH₂CH₂CH₃), butoxy (—OCH₂CH₂CH₂CH₃), 1-methylethoxy (—OCH(CH₃)₂), and 1,1-dimethylethoxy (—OC(CH₃)₃).

The term “aryl” as used herein and unless specified otherwise, either alone or in combination with another radical, is intended to mean a carbocyclic aromatic monocyclic group containing 6 carbon atoms which may be further fused to a second 5- or 6-membered carbocyclic group which may be aromatic, saturated or unsaturated. “Aryl” includes, but is not limited to, phenyl, indanyl, indenyl, 1-naphthyl, 2-naphthyl, tetrahydronaphthyl and dihydronaphthyl.

The terms “arylalkyl” or “aryl(C₁₋ₙ)alkyl” as used herein and unless specified otherwise, wherein n is an integer, either alone or in combination with another radical, are intended to mean a saturated, acyclic alkyl radical having 1 to n carbon atoms as defined above which is itself substituted with an aryl radical as defined above. Examples of arylalkyl include, but are not limited to, phenylmethyl (benzyl), 1-phenylethyl, 2-phenylethyl and phenylpropyl. When an arylalkyl group is substituted, it is understood that substituents may be attached to either the aryl or the alkyl portion thereof or both, unless specified otherwise, such that the substitution would give rise to a chemically stable compound, such as are recognized by those skilled in the art.

The term “heteroatom” as used herein and unless specified otherwise is intended to mean O, S or N.

The term “carbocycle” as used herein and unless specified otherwise, either alone or in combination with another radical, is intended to mean a 3- to 8-membered saturated, unsaturated or aromatic cyclic radical in which all of the ring members are carbon atoms, and which may be fused to one or more 3- to 8-membered saturated, unsaturated or aromatic carbocyclic groups. When a carbocycle is substituted, it is understood that substituents may be attached to any carbon atom which would otherwise bear a hydrogen atom, unless specified otherwise, such that the substitution would give rise to a chemically stable compound, such as are recognized by those skilled in the art.

The term “heterocycle” as used herein and unless specified otherwise, either alone or in combination with another radical, is intended to mean a 4- to 10-membered saturated, unsaturated or aromatic monocyclic heterocycle containing from 1 to 4 heteroatoms each independently selected from O, N and S which is optionally fused to one or more other cycle, including a carbocycle, a heterocycle or any other cycle; or a monovalent radical derived by removal of a hydrogen atom therefrom. Examples of such heterocycles include, but are not limited to, azetidine, pyrrolidine, tetrahydrofuran, tetrahydrothiophene, thiazolidine, oxazolidine, pyrrole, thiophene, furan, pyrazole, imidazole, isoxazole, oxazole, isothiazole, thiazole, triazole, tetrazole, piperidine, piperazine, azepine, diazepine, pyran, 1,4-dioxane, 4-morpholine, 4-thiomorpholine, pyridine, pyridine-N-oxide, pyridazine, pyrazine, pyrimidine, indole, isoindole, benzimidazole, benzothiophene, benzofuran, benzopyran, benzodioxole, benzodioxane, benzothiazole, quinoline, isoquinoline, and naphthyridine, and saturated, unsaturated and aromatic derivatives thereof.

The term “polyoxyalkylene” as used herein and unless specified otherwise, either alone or in combination with another radical, is intended to mean a radical of the formula —(O—(C(R^{a})(R^{b}))ₙ)ₘ—, wherein n is an integer from 1 to 6, m is an integer from 1 to 30, and R^{a} and R^{b} are each independently in each instance selected from H and saturated (C₁₋₆)alkyl. In at least one embodiment, n is an integer from 1 to 3. In at least one embodiment, n is 2. Examples of polyoxyalkylene include but are not limited to polyoxyethylene, wherein n is 2 and R^{a} and R^{b} are each H, and polyoxypropylene, wherein n is 2, one instance of R^{a} is a methyl group, and R^{b} and the other instance of R^{a} are each H. In at least one embodiment, when polyoxyalkylene is polyoxyethylene, m is an integer from 1 to 30. In at least one embodiment, when polyoxyalkylene is polyoxypropylene, m is an integer from 1 to 10. As used herein, the term “hydroxyalkylpolyoxyalkylene” is intended to mean a radical of the formula HO—(C(R^{a})(R^{b}))ₙ—(O—(C(R^{a})(R^{b}))ₙ)ₘ—, wherein n is an integer from 1 to 6, m is an integer from 1 to 30, and R^{a} and R^{b} are each independently in each instance selected from H and saturated (C₁₋₆)alkyl.

The term “surfactant headgroup” as used herein and unless specified otherwise is intended to mean a polar or ionic hydrophilic group which interacts strongly with water and which is solvated via dipole-dipole or ion-dipole interactions. Examples of surfactant headgroups include but are not limited to hydroxy, sulfonate, sulfate, carboxylate, phosphonate, phosphate, and primary, secondary, tertiary or quaternary ammonium. It will be clear to the skilled person that when a surfactant headgroup is a charged group, a suitable counterion will also be present. When the surfactant headgroup is an anionic group, suitable counterions are cations, including but not limited to metal cations and optionally substituted ammonium cations. When the surfactant headgroup is a cationic group, suitable counterions are anions, including but not limited to halide, hydroxide, nitrate, sulfate, sulfonate, carbonate, carboxylate, phosphate and phosphonate anions. The surfactant headgroup can also include a linker which connects the polar or ionic group to the remainder of the surfactant molecule. Such linkers can have from 1 to 10 atoms each independently selected from C, O, N and S, in addition to any attached hydrogen atoms.

The term “hydrophobic group” as used herein and unless specified otherwise is intended to mean a group which is hydrophobic or non-polar and which interacts only very weakly with water, or is a polyoxyalkylene or hydroxypolyoxyalkylene group. Examples of hydrophobic groups include but are not limited to alkyl, aryl, arylalkyl, polyoxyalkylene and hydroxypolyoxyalkylene groups, including but not limited to alkyl, aryl, arylalkyl, polyoxyalkylene and hydroxypolyoxyalkylene groups which are unsubstituted or are substituted with non-polar substituents.

### DETAILED DESCRIPTION OF THE INVENTION

One aspect of the present invention provides a compound of formula IA

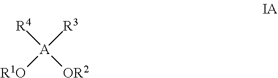

wherein A is a core derived from an organic polyhydroxy compound;

R¹ and R² are each independently a hydrophobic group; and

R³ and R⁴ are each independently a surfactant headgroup.

In the following embodiments, groups and substituents of the compounds of formula IA according to the invention are described in detail.

Core A can be a core derived from any organic compound containing at least two hydroxy groups. Suitable organic compounds include but are not limited to polyalcohols, including but not limited to diols, triols, including but not limited to glycerol, tetraols, including but not limited to pentaerythritol, and polyols, including but not limited to polyglycerols and polypentaerythritols; sugars; and sugar derivatives, including but not limited to sugar alcohols, sugar acids, alkyl glycosides, oligosaccharides and polysaccharides. In at least one embodiment, core A is derived from methyl glucoside, a polyglycerol or pentaerythritol.

In at least one embodiment, wherein A is a core derived from pentaerythritol, the present invention provides a compound of formula I

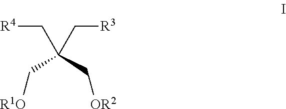

wherein R¹ and R² are each independently a hydrophobic group; and

R³ and R⁴ are each independently a surfactant headgroup.

### R¹ and R²

In at least one embodiment, R¹ is identical to R².

In at least one embodiment, R¹ and R² are each independently a hydrophobic group selected from (C₁₋₂₄)alkyl, aryl(C₁₋₂₄)alkyl and (C₁₋₂₀)hydroxyalkylpolyoxyalkylene; wherein the aryl(C₁₋₂₄)alkyl is optionally substituted with from one to three (C₁₋₂₄)alkyl groups; and wherein the (C₁₋₂₄)alkyl is optionally substituted with hydroxyl, (C₁₋₂₄)alkoxy, (C₁₋₂₄)alkyl-C(═O)NH—, or (C₁₋₂₄)alkyl-NHC(═O)—.

In at least one embodiment, R¹ and R² are each independently selected from (C₁₋₂₄)alkyl, aryl(C₁₋₂₄)alkyl and (C₁₋₁₄)hydroxyalkylpolyoxyalkylene; wherein the (C₁₋₂₄)alkyl is optionally substituted with hydroxyl and the aryl(C₁₋₂₄)alkyl is optionally substituted with (C₁₋₂₄)alkyl.

In at least one embodiment, R¹ and R² are each independently selected from (C₈₋₁₄)alkyl, aryl(C₁₋₆)alkyl substituted with (C₈₋₁₂)alkyl, and (C₁₋₆)alkyl substituted with hydroxyl.

In at least one embodiment, at least one of R¹ and R² is (C₁₋₂₄)alkyl.

In at least one embodiment, at least one of R¹ and R² is (C₈₋₁₄)alkyl.

In at least one embodiment, at least one of R¹ and R² is selected from octyl, nonyl, decyl, undecyl, dodecyl, tridecyl and tetradecyl.

In at least one embodiment, at least one of R¹ and R² is aryl(C₁₋₆)alkyl substituted with (C₈₋₁₂)alkyl.

In at least one embodiment, at least one of R¹ and R² is phenyl-CH₂-substituted with (C₈₋₁₂)alkyl.

In at least one embodiment, at least one of R¹ and R² is phenyl-CH₂-substituted with a group selected from octyl, nonyl, decyl, undecyl and dodecyl.

In at least one embodiment, at least one of R¹ and R² is (C₁₋₆)alkyl substituted with hydroxyl.

In at least one embodiment, at least one of R¹ and R² is selected from hydroxyethyl and hydroxypropyl.

### R³ and R⁴

In at least one embodiment, R³ is identical to R⁴.

In at least one embodiment, R³ and R⁴ are each independently a surfactant headgroup selected from —OH, —SO₃⁻, —(C₁₋₆)alkyl-SO₃⁻, —O(C₁₋₆)alkyl-SO₃⁻, —OSO₃⁻, —(C₁₋₆)alkyl-OSO₃⁻, —O(C₂₋₆)alkyl-OSO₃⁻, —COO⁻, —(C₁₋₆)alkyl-COO⁻, —O(C₁₋₆)alkyl-COO⁻, —PO₃²⁻, —(C₁₋₆)alkyl-PO₃²⁻, —O(C₁₋₆)alkyl-PO₃²⁻, —PO₃H⁻, —(C₁₋₆)alkyl-PO₃H⁻, —O(C₁₋₆)alkyl-PO₃H⁻, —OPO₃²⁻, —(C₁₋₆)alkyl-OPO₃²⁻, —O(C₂₋₆)alkyl-OPO₃²⁻, —OPO₃H⁻, —(C₁₋₆)alkyl-OPO₃H⁻, —O(C₂₋₆)alkyl-OPO₃H⁻, —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance H, —(C₁₋₆)alkyl, —(C₂₋₆)alkyl-OH, —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻, —(C₁₋₆)alkyl-COO⁻, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

It will be clear to the skilled person that when R³ or R⁴ is a charged group, a suitable counterion will also be present. When at least one of R³ and R⁴ is an anionic group, suitable counterions are cations, including but not limited to metal cations and optionally substituted ammonium cations. When at least one of R³ and R⁴ is a cationic group, suitable counterions are anions, including but not limited to halide, hydroxide, nitrate, sulfate, sulfonate, carbonate, carboxylate, phosphate and phosphonate anions. It is also contemplated that one of R³ and R⁴ can be an anionic group and the other of R³ and R⁴ can be a cationic group, such that a zwitterionic or amphoteric structure results and further counterions are not necessary. Alternatively, at least one of R³ and R⁴ can contain both an anionic group and a cationic group, such that the at least one of R³ and R⁴ is itself zwitterionic.

In at least one embodiment, at least one of R³ and R⁴ is an anionic surfactant headgroup selected from —SO₃⁻, —(C₁₋₆)alkyl-SO₃⁻, —O(C₁₋₆)alkyl-SO₃⁻, —OSO₃⁻, —(C₁₋₆)alkyl-OSO₃⁻, —O(C₂₋₆)alkyl-OSO₃⁻, —COO⁻, —(C₁₋₆)alkyl-COO⁻, —O(C₁₋₆)alkyl-COO⁻, —PO₃²⁻, —(C₁₋₆)alkyl-PO₃²⁻, —O(C₁₋₆)alkyl-PO₃²⁻, —PO₃H⁻, —(C₁₋₆)alkyl-PO₃H⁻, —O(C₁₋₆)alkyl-PO₃H⁻, —OPO₃²⁻, —(C₁₋₆)alkyl-OPO₃²⁻, —O(C₂₋₆)alkyl-OPO₃²⁻, —OPO₃H⁻, —(C₁₋₆)alkyl-OPO₃H⁻ and —O(C₂₋₆)alkyl-OPO₃H⁻.

In at least one embodiment, at least one of R³ and R⁴ is an anionic surfactant headgroup selected from —SO₂⁻, —(C₁₋₃)alkyl-SO₃⁻, —O(C₁₋₃)alkyl-SO₃⁻, —OSO₃⁻, —(C₁₋₃)alkyl-OSO₃⁻, —O(C₂₋₃)alkyl-OSO₃⁻, —OPO₃²⁻, —(C₁₋₃)alkyl-OPO₃²⁻, —O(C₂₋₃)alkyl-OPO₃²⁻, —COO⁻, —(C₁₋₃)alkyl-COO⁻, and —O(C₁₋₃)alkyl-COO⁻.

In at least one embodiment, at least one of R³ and R⁴ is an anionic surfactant headgroup selected from —OSO₃⁻, —OCH₂CH₂OSO₃⁻, —OCH₂CH₂CH₂OSO₃⁻, —OPO₃²⁻, —OCH₂CH₂OPO₃²⁻, —OCH₂CH₂CH₂OPO₃²⁻, —COO⁻, —OCH₂COO⁻, —OCH₂CH₂COO⁻, —OCH₂CH₂CH₂COO⁻, —OCH₂CH₂SO₃⁻ and —OCH₂CH₂CH₂SO₃⁻.

In at least one embodiment, at least one of R³ and R⁴ is a cationic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance H, —(C₁₋₆)alkyl, —(C₂₋₆)alkyl-OH, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a cationic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance —(C₁₋₆)alkyl, —(C₂₋₆)alkyl-OH, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a cationic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance —(C₁₋₆)alkyl, —(C₂₋₆)alkyl-OH, or two of R⁵, R⁶ and R⁷ are joined, together with the N atom to which they are attached, to form a 4-, 5-, 6-, 7- or 8-membered heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a cationic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance —(C₁₋₆)alkyl, —(C₂₋₆)alkyl-OH, or two of R⁵, R⁶ and R⁷ are joined, together with the N atom to which they are attached, to form a 5- or 6-membered heterocycle containing one N heteroatom, the heterocycle being optionally substituted with from one to three substituents each independently selected from (C₁₋₆)alkyl and aryl.

In at least one embodiment, at least one of R³ and R⁴ is a cationic surfactant headgroup selected from —NH₃⁺ (ammonium), N-methylammonium, N,N-dimethylammonium, N,N,N-trimethylammonium, N-ethylammonium, N,N-diethylammonium, N,N,N-triethylammonium, N,N,N-ethyldimethylammonium, N,N,N-diethylmethylammonium, N-(2-hydroxyethyl)ammonium, N,N-di-(2-hydroxyethyl)ammonium, N,N,N-tri-(2-hydroxyethyl)ammonium, ammoniomethyl, N-methylammoniomethyl, N,N-dimethylammoniomethyl, N,N,N-trimethylammoniomethyl, N-ethylammoniomethyl, N,N-diethylammoniomethyl, N,N,N-triethylammoniomethyl, N,N,N-ethyldimethylammoniomethyl, N,N,N-diethylmethylammoniomethyl, N-(2-hydroxyethyl)ammoniomethyl, N,N-di(2-hydroxyethyl)ammoniomethyl, N,N,N-tri-(2-hydroxyethyl)ammoniomethyl, 2-ammonioethoxy, 2-(N-methylammonio)ethoxy, 2-(N,N-dimethylammonio)ethoxy, 2-(N,N,N-trimethylammonio)ethoxy, 2-(N-ethylammonio)ethoxy, 2-(N,N-diethylammonio)ethoxy, 2-(N,N,N-triethylammonio)ethoxy, 2-(N,N,N-ethyldimethylammonio)ethoxy, 2-(N,N,N-diethylmethylammonio)ethoxy, 2-{N-(2-hydroxyethyl)ammonio}ethoxy, 2-{N,N-di(2-hydroxyethyl)ammonio}ethoxy, 2-{N,N,N-tri-(2-hydroxyethyl)ammonio}ethoxy, 3-ammoniopropanoxy, 3-(N-methylammonio)propanoxy, 3-(N,N-dimethylammonio)propanoxy, 3-(N,N,N-trimethylammonio)propanoxy, 3-(N-ethylammonio)propanoxy, 3-(N,N-diethylammonio)propanoxy, 3-(N,N,N-triethylammonio)propanoxy, 3-(N,N,N-ethyldimethylammonio)propanoxy, 3-(N,N,N-diethylmethylammonio)propanoxy, 2-{N-(2-hydroxyethyl)ammonio}propanoxy, 3-{N,N-di(2-hydroxyethyl)ammonio}propanoxy and 3-{N,N,N-tri-(2-hydroxyethyl)ammonio}propanoxy.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻ or —(C₁₋₆)alkyl-COO⁻ and the remaining two of R⁵, R⁶ and R⁷ are each independently in each instance H, —(C₁₋₆)alkyl, —(C₂₋₆)alkyl-OH, or the remaining two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻ or —(C₁₋₆)alkyl-COO⁻ and the remaining two of R⁵, R⁶ and R⁷ are each independently in each instance —(C₁₋₆)alkyl, —(C₂₋₆)alkyl-OH, or the remaining two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻ or —(C₁₋₆)alkyl-COO⁻ and the remaining two of R⁵, R⁶ and R⁷ are each independently in each instance —(C₁₋₆)alkyl, —(C₂₋₆)alkyl-OH, or the remaining two of R⁵, R⁶ and R⁷ are joined, together with the N atom to which they are attached, to form a 4-, 5-, 6-, 7- or 8-membered heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻ or —(C₁₋₆)alkyl-COO⁻ and the remaining two of R⁵, R⁶ and R⁷ are each independently in each instance —(C₁₋₆)alkyl, —(C₂₋₆)alkyl-OH, or the remaining two of R⁵, R⁶ and R⁷ are joined, together with the N atom to which they are attached, to form a 5- or 6-membered heterocycle containing one N heteroatom, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-COO⁻ and the remaining two of R⁵, R⁶ and R⁷ are each independently in each instance H, —(C₁₋₆)alkyl, —(C₂₋₆)alkyl-OH, or the remaining two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-COO⁻ and the remaining two of R⁵, R⁶ and R⁷ are each independently in each instance —(C₁₋₆)alkyl, —(C₂₋₆)alkyl-OH, or the remaining two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-COO⁻ and the remaining two of R⁵, R⁶ and R⁷ are each independently in each instance —(C₁₋₆)alkyl, —(C₂₋₆)alkyl-OH, or the remaining two of R⁵, R⁶ and R⁷ are joined, together with the N atom to which they are attached, to form a 4-, 5-, 6-, 7- or 8-membered heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

In at least one embodiment, at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein one of R⁵, R⁶ and R⁷ is —(C₁₋₆)alkyl-COO⁻ and the remaining two of R⁵, R⁶ and R⁷ are each independently in each instance —(C₁₋₆)alkyl, —(C₂₋₆)alkyl-OH, or the remaining two of R⁵, R⁶ and R⁷ are joined, together with the N atom to which they are attached, to form a 5- or 6-membered heterocycle containing one N heteroatom, the heterocycle being optionally substituted with from one to three substituents each independently selected from (C₁₋₆)alkyl and aryl.

In at least one embodiment, at least one of R³ and R⁴ is a zwitterionic surfactant headgroup selected from N-carboxymethylammonium, N,N-carboxymethylmethylammonium, N,N,N-carboxymethyldimethylammonium, N-carboxymethylammoniomethyl, N,N-carboxymethylmethylammoniomethyl, N,N,N-carboxymethyldimethylammoniomethyl, 2-(N-carboxymethylammonio)ethoxy, 2-(N,N-carboxymethylmethylammonio)ethoxy, 2-(N,N,N-carboxymethyldimethylammonio)ethoxy, 3-(N-carboxymethylammonio)propanoxy, 3-(N,N-carboxymethylmethylammonio)propanoxy, and 3-(N,N,N-carboxymethyldimethylammonio)propanoxy

In at least one embodiment, the present invention provides a compound of formula I

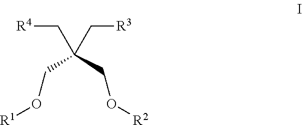

wherein R¹ and R² are each independently a hydrophobic group selected from (C₁₋₂₄)alkyl, aryl(C₁₋₂₄)alkyl and (C₁₋₂₀)hydroxyalkylpolyoxyalkylene; wherein the aryl(C₁₋₂₀)alkyl is optionally substituted with from one to three (C₁₋₂₄)alkyl groups; and wherein the (C₁₋₂₄)alkyl is optionally substituted with hydroxyl, (C₁₋₂₄)alkoxy, (C₁₋₂₄)alkyl-C(═O)NH—, or (C₁₋₂₄)alkyl-NHC(═O)—; and
   R³ and R⁴ are each independently a surfactant headgroup selected from —OH, —SO₃⁻, —(C₁₋₆)alkyl-SO₃⁻, —O(C₁₋₆)alkyl-SO₃⁻, —OSO₃⁻, —(C₁₋₆)alkyl-OSO₃⁻, —O(C₂₋₆)alkyl-OSO₃⁻, —COO⁻, —(C₁₋₆)alkyl-COO⁻, —O(C₁₋₆)alkyl-COO⁻, —PO₃²⁻, —O(C₁₋₆)alkyl-PO₃²⁻, —PO₃H⁻, —(C₁₋₆)alkyl-PO₃H⁻, —O(C₁₋₆)alkyl-PO₃H⁻, —OPO₃²⁻, —(C₁₋₆)alkyl-OPO₃²⁻, —O(C₂₋₆)alkyl-OPO₃²⁻, —OPO₃H⁻, —(C₁₋₆)alkyl-OPO₃H⁻, —O(C₂₋₆)alkyl-OPO₃H⁻, —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
   wherein R⁵, R⁶ and R⁷ are each independently in each instance H, —(C₁₋₆)alkyl, —(C₂₋₆)alkyl-OH, —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻, —(C₁₋₆)alkyl-COO⁻, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

The compounds of the present invention are useful as surfactants. Such surfactants can be used as components of fluids used in the petroleum industry for the production or recovery of petroleum from petroleum-bearing formations, in applications including but not limited to drilling, completion, work over or servicing of oil and gas wells, treatment of oil and gas bearing formations, enhancement of production from oil and gas bearing formations, bioremediation, hydraulic fracturing, and well stimulation, including but not limited to chemical flooding oil recovery and foam flooding oil recovery, and other methods of secondary and tertiary oil and heavy oil recovery. In at least one embodiment, compounds according to the present invention having zwitterionic or amphoteric headgroups, or which exhibit the property of viscoelasticity, can be friction pressure reducing agents for the preparation of well stimulation fluids, including but not limited to those used in fracturing and acidizing fluids. In addition, in at least one embodiment, the compounds of the present invention can be used as agents for shale stabilization, including but not limited to when drilling in gumbo and in young, easily hydrated rock formations. In at least one embodiment, the compounds of the present invention can also be used as super shale inhibitors in water-based fluid systems where the use of potassium salts and amine salts of the ethylene diamine type have proven ineffective, and/or are prohibited due to environmental constraints.

Another aspect of the present invention provides a fluid for use in the production or recovery of petroleum from petroleum-bearing formations, including but not limited to drilling, completion, work over or servicing of oil and gas wells, treatment of oil and gas bearing formations, enhancement of production from oil and gas bearing formations, bioremediation, hydraulic fracturing, and well stimulation, including but not limited to chemical flooding oil recovery and foam flooding oil recovery, the fluid comprising a compound according to the present invention as defined herein, a base fluid and optionally at least one chemical additive. In at least one embodiment, when the fluid is used for hydraulic fracturing, the compound according to the present invention can advantageously form micelles which are worm-like in nature, thereby acting to impart the property of viscoelasticity to the fluid.

According to another aspect of the present invention, there is provided a method for using a fluid comprising a compound according to the present invention as defined herein in the production or recovery of petroleum from petroleum-bearing formations, including but not limited to drilling, completion, work over or servicing of oil and gas wells, treatment of oil and gas bearing formations, enhancement of production from oil and gas bearing formations, bioremediation, hydraulic fracturing, and well stimulation, including but not limited to chemical flooding oil recovery and foam flooding oil recovery.

In at least one embodiment, the fluid is a fabricated fluid suitable for use in the drilling, completion, work over or servicing of oil and gas wells, treatment of oil and gas bearing formations, enhancement of production from oil and gas bearing formations, hydraulic fracturing, and well stimulation, including but not limited to chemical flooding oil recovery and foam flooding oil recovery.

According to yet another aspect of the present invention, there is also provided a method of preparing a fluid for use in drilling, completion, work over or servicing of oil and gas wells, treatment of oil and gas bearing formations, enhancement of production from oil and gas bearing formations, hydraulic fracturing, or well stimulation, including but not limited to chemical flooding oil recovery and foam flooding oil recovery, the method comprising adding a predetermined amount of a compound according to the present invention to a base fluid and mixing the compound according to the present invention and the base fluid. In at least one embodiment the base fluid comprises at least one chemical additive.

Suitable base fluids can be chosen by the skilled person based at least partly on the specific purpose of the fluid, as will be appreciated by the person of skill in the art, and include, but are not limited to, aqueous base fluids and non-aqueous base fluids, including but not limited to hydrocarbon base fluids, such as, for example, diesel oil, and synthetic base fluids.

Chemical additives which may be added to a fluid, including but not limited to a drilling fluid, for use in the production or recovery of petroleum from petroleum-bearing formations include but are not limited to weight materials, fluid loss control agents, bridging agents, lubricants, anti-bit balling agents, corrosion inhibition agents, surfactants and suspending agents. Such components can be added in the concentrations needed to adjust the rheological and functional properties of the drilling fluid appropriate to the drilling conditions, as would be apparent to the skilled person. Suitable examples of each of these additional components are well known to the person of skill in the art.

Weight materials are inert, high-density particulate materials used to increase the density of the drilling fluid. Suitable weight materials are known in the art and include, but are not limited to such examples as calcium carbonate, magnesium carbonate, iron oxide, barite, hematite, ilmenite, water-soluble organic and inorganic salts, and mixtures thereof.

Fluid loss control agents are added to drilling fluids to help prevent or reduce fluid loss during the drilling process. Suitable examples of fluid loss control agents include but are not limited to synthetic organic polymers including but not limited to polyacrylate; biopolymers including but not limited to starches, modified starches and modified celluloses; modified lignite; lignosulfonate; silica; mica; calcite; and mixtures thereof.

Bridging agents are materials added to a drilling fluid to bridge across pores and fractures of exposed rock, to seal formations, and to aid in forming a filter cake. Advantageously, bridging agents are removable from the wellbore after drilling is complete, to facilitate recovery when the well enters production. Suitable examples of bridging agents include but are not limited to magnesium oxide, manganese oxide, calcium oxide, lanthanum oxide, cupric oxide, zinc oxide, magnesium carbonate, calcium carbonate, zinc carbonate, calcium hydroxide, manganese hydroxide, suspended salts, oil-soluble resins, mica, nutshells, fibers and mixtures thereof.

Lubricants are used to lower friction, including but not limited to torque and drag in the wellbore, and to lubricate unsealed bit bearings. Suitable examples of lubricants include but are not limited to plastic beads, glass beads, nut hulls, graphite, oils, synthetic fluids, glycols, modified vegetable oils, fatty-acid soaps, surfactants and mixtures thereof.

Anti-bit balling agents are used to prevent compaction and adherence of drill cuttings to the cutting surfaces of the drill bit, causing fouling and a reduction of drill performance. Suitable examples of anti-bit balling agents include but are not limited to glycols, surfactants and mixtures thereof.

Corrosion inhibition agents are used to protect the metal components of the drill from corrosion caused by contact with materials such as water, carbon dioxide, biological deposits, hydrogen sulfide and acids. Suitable examples of corrosion inhibition agents include but are not limited to amines, zinc compounds, chromate compounds, cyanogen-based inorganic compounds, sodium nitrite based compounds and mixtures thereof.

Surfactants are surface active agents that can function as emulsifiers, dispersants, oil-wetters, water-wetters, foamers and defoamers. Suitable examples of surfactants include but are not limited to anionic surfactants, cationic surfactants, zwitterionic surfactants, nonionic surfactants, and suitable mixtures of any of the above known to one skilled in the art.

Suspending agents alter the rheological and viscosity properties of the drilling fluid, thereby allowing small solid particles to remain suspended in the fluid. Suitable examples of suspending agents include but are not limited to clays, biopolymers, gums, silicates, fatty acids, synthetic polymers and mixtures thereof.

The compounds of the present invention can also be used to accelerate bioremediation, or the bacterial removal of oil from cuttings formed during the drilling of oil wells. Without being bound by theory, it is believed that the present compounds can aid the removal of grease and oil from the cuttings due to their wetting ability and their ability to lower surface and interfacial tension. In this way, these compounds, when added to drilling fluids, can act to increase the bioavailability of the oil to the bacteria used in the remediation cycle, so as to facilitate bioremediation of the cuttings. Therefore, according to another aspect of the present invention, there is provided a method for the bioremediation of cuttings produced during the drilling of a well bore from a fluid used to transport the cuttings from the bottom of said well bore to the surface, wherein the fluid comprises an experimentally determined amount of a compound according to the present invention as defined herein.

According to yet another aspect of the present invention, there is also provided a method of fracturing an underground hydrocarbon bearing formation penetrated by a well bore, comprising the steps of injecting a stream of fluid comprising a compound according to the present invention into the formation at a pressure selected to cause the formation of at least one fracture in the formation. In at least one embodiment, the compound according to the invention can form micelles which are worm-like in nature, thereby acting to impart the property of viscoelasticity to the fluid. In at least one embodiment, the fluid further comprises at least one proppant, used to prop open the fracture. Suitable proppants include but are not limited to graded sand, bauxite, ceramics, and nut hulls.

According to yet another aspect of the present invention, there is also provided a method of reducing turbulent flow in a fluid flowing past a stationary object, the method comprising adding a compound according to the present invention to the fluid. In at least one embodiment, the stationary object is a pipe wall, an earth formation, a boat bottom or a surface encountered in central heating distribution.

Compositions containing compounds of the present invention can also be envisioned to have applications in other industries besides the petroleum industry. For example, the compounds of the present invention would be suitable in cleansing compositions or detergent compositions, including, but not limited to hair shampoos, hair conditioners, cream cleansers, body washes, dishwashing liquids, dishwashing powders and laundry detergents. Detergent compositions containing surfactants according to the present invention can be prepared or used in any known forms, e.g. in solid, liquid, cream, foam, or powder form. Such detergent compositions can, by someone skilled in the art, be made into any of a number of well known desirable forms such as bars, granules, flakes, liquids, and tablets. The detergent formulations incorporating or embodying the novel surfactants of the present invention may contain any of the usual adjuvants, diluents and additives, including but not limited to perfumes, antitarnishing agents, anti-redeposition agents, anti-bacterial agents, dyes, fluorescent agents, suds builders, suds depressors, foam stabilizers and co-surfactants. Suitable co-surfactants can include other well known natural soaps or synthetic anionic, non-ionic, zwitterionic, or amphoteric amphiphiles. As a non-limiting example, contemplated detergent formulations would comprise blending a surfactant of the present invention with a detergency builder. The amount and type of the surfactant of the present invention usefully present in the detergent formulations will depend on the application, and would be determined experimentally by any of a number of known methods. It is to be understood that contemplated detergent formulations comprising at least one surfactant according to the present invention are not limited to any particular method of preparation.

Emulsion compositions containing the surfactants of the present invention are also contemplated. In this application, the amount of surfactant may vary, and can be determined by any of a number of standard techniques known by those skilled in the art. The emulsion composition of the invention can contain the surfactant, water, and oil components usually blended into an emulsion composition. Suitable oil components include but are not limited to liquid oils, solid oils, waxes, hydrocarbon oils, higher fatty acids, higher alcohols, synthetic ester oils, silicone oils, etc. Where necessary, the emulsion composition may additionally contain other surfactants and additives which are usually blended into an emulsion composition. Suitable other surfactants include but are not limited to anionic surfactants, amphoteric surfactants, nonionic surfactants (lipophilic, hydrophilic), and cationic surfactants. Suitable additives include but are not limited to humectants, powdery components, water-soluble polymers, viscosity improvers, UV absorbents, metal ion sequestering agents, lower alcohols, polyhydric alcohols, saccharides (monosaccharides, oligosaccharides, and polysaccharides), amino acids, organic amines, pH controlling agents, antioxidants, auxiliary antioxidants, preservatives, antiphlogistics, whitening agents, extracts, activating agents, circulation stimulants, anti-seborrhea agents, and anti-inflammatory agents.

Suitable formulation of the surfactants of the present invention into an emulsion composition by someone of skill in the art will ensure a good emulsion state. The emulsion compositions containing the invented surfactants can be used in any known forms such as creams, liquids, or gels. The emulsion composition can suit any of a number of known applications containing said emulsion, including but not limited to known cosmetics (creams, milky lotions, and serums), pharmaceuticals, medicated cosmetics, and foods.

The surfactants of the present invention can also be used for a number of other well-known surfactant applications, including but not limited to scouring agents, foaming agents, defoamers, demulsifying agent, dispersants, wetting agents, dissolving agents, lustering agents, delustering agents, softening agents, water repellents, flame repellents, antistatic agents, and flotation agents.

### Synthetic Methodology

Compounds of general formula I

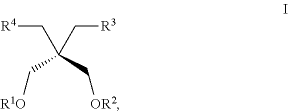

wherein R¹, R², R³ and R⁴ are as defined herein, are conveniently prepared by the procedures illustrated in the following schemes. It will be apparent to the skilled person that other procedures well known in the art may be used in the preparation of the present compounds. The skilled person will also recognize that the procedures described herein will also be applicable to the synthesis of compounds of the formula IA

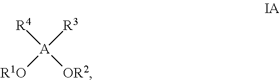

wherein A is a core derived from other organic polyhdroxy compounds.

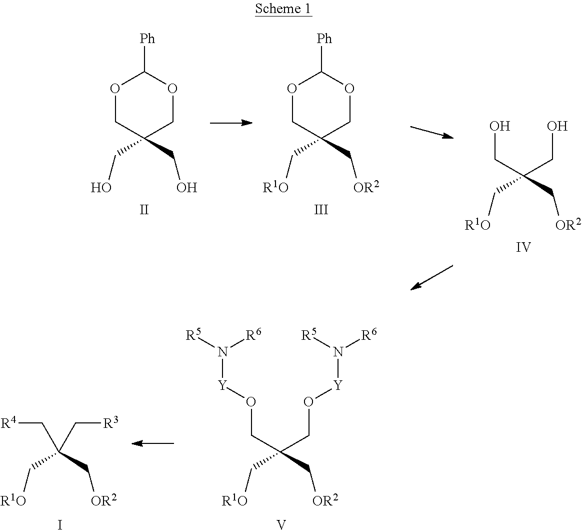

Mono-O-benzylidenepentaerythritol (II) is prepared by the method of Issidorides and Gulen (Organic Syntheses Collected Volume IV, Rabjohn, N., Ed.; John Wiley and Sons: New York, 1963; pp 679-681.) Treatment of mono-O-benzylidenepentaerythritol with sodium hydride followed by an alkylating agent, including but not limited to an alkyl halide or an arylalkyl halide, under well-known conditions, provides intermediate III, wherein R¹ and R² are alkyl or arylalkyl. Hydrogenolysis of intermediate III provides intermediate IV. It will be apparent to the skilled person that other well-known protecting groups, including but not limited to other acetals, can be used in the preparation of intermediate IV.

Intermediate IV is transformed to intermediate V by reaction with sodium hydride, followed by a reagent of formula X—Y—N(R⁵)(R⁶), wherein X is a leaving group, Y is (C₂₋₆)alkyl, and R⁵ and R⁶ are as defined herein. Such a reagent can be in the form of a salt, including but not limited to the hydrochloride salt. Intermediate V can be treated with an acid, including but not limited to hydrochloric acid, to give compounds of formula I wherein R¹ and R² are alkyl or arylalkyl and R³ and R⁴ are —OY—N(R⁵)(R⁶)(R⁷)⁺, wherein R⁷ is H. Alternatively, intermediate IV can be allowed to react with an alkylating agent of formula R^{7a}—X, wherein X is a leaving group and R^{7a} is R⁷ as defined herein or a group which may be subsequently transformed to R⁷, to give compounds of formula I wherein R¹ and R² are alkyl or arylalkyl and R³ and R⁴ are —OY—N(R⁵)(R⁶)(R⁷)⁺. It will be clear to the person of skill in the art that when R⁷ is a —(C₁₋₆)alkyl-COO⁻ group, R^{7a} can have the formula —(C₁₋₆)alkyl-COOP, wherein P is a protecting group, including but not limited to an alkyl group, which may readily be removed, by well known procedures, including but not limited to hydrolysis, to form R⁷.

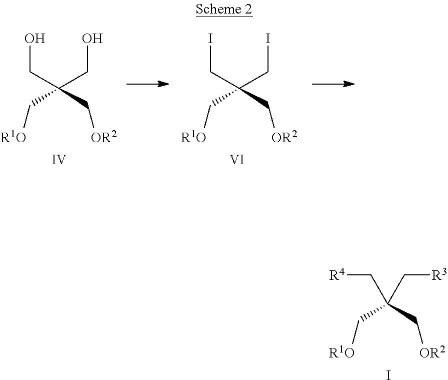

Reaction of intermediate IV with I₂ and PPh₃ under well known conditions, or using other well-known procedures, provides intermediate VI, wherein R¹ and R² are as defined herein. Intermediate VI can be allowed to react with an amine of formula HN(R⁵)(R⁶), wherein R⁵ and R⁶ are as defined herein, followed by acidification, to give compounds of formula I wherein R¹ and R² are alkyl or arylalkyl, and R³ and R⁴ are —NH(R⁵)(R⁶)⁺. Alternatively, intermediate VI can be allowed to react with an amine of formula HN(R⁵)(R⁶), wherein R⁵ and R⁶ are as defined herein, followed by acidification or alkylation with an alkylating agent of formula R^{7a}—X, wherein X is a leaving group and R^{7a} is R⁷ as defined herein or a group which may be subsequently transformed to R⁷, to give compounds of formula I wherein R¹ and R² are alkyl or arylalkyl, and R³ and R⁴ are —N(R⁵)(R⁶)(R⁷)⁺.

Furthermore, intermediate VI can be transformed to compounds of formula I by the procedure illustrated in Scheme 3.

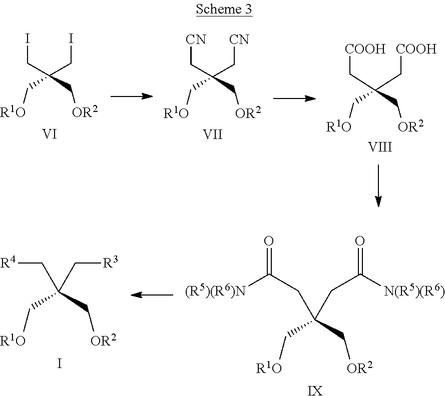

Reaction of intermediate VI with KCN provides intermediate VII, which can be hydrolyzed by procedures well known in the art, including but not limited to hydrolysis in the presence of NaOH, to give intermediate VIII. Intermediate VIII is allowed to react with an amine of formula HN(R⁵)(R⁶), wherein R⁵ and R⁶ are as defined herein, under well known conditions, including but not limited to reaction in the presence of 1-hydroxybenzotriazole and N-(3-dimethylaminopropyl)-N′-ethylcarbodiimide hydrochloride to give intermediate IX. Reduction of the amide functionality of intermediate IX using well known reagents including but not limited to LiAlH₄, provides compounds of formula I wherein R¹ and R² are alkyl or arylalkyl and R³ and R⁴ are —CH₂N(R⁵)(R⁶). Acidification or alkylation with an alkylating agent of formula R^{7a}—X, wherein X is a leaving group and R^{7a} is R⁷ as defined herein or a group which may be subsequently transformed to R⁷, provides compounds of formula I wherein R¹ and R² are alkyl or arylalkyl, and R³ and R⁴ are —CH₂N(R⁵)(R⁶)(R⁷)⁺.

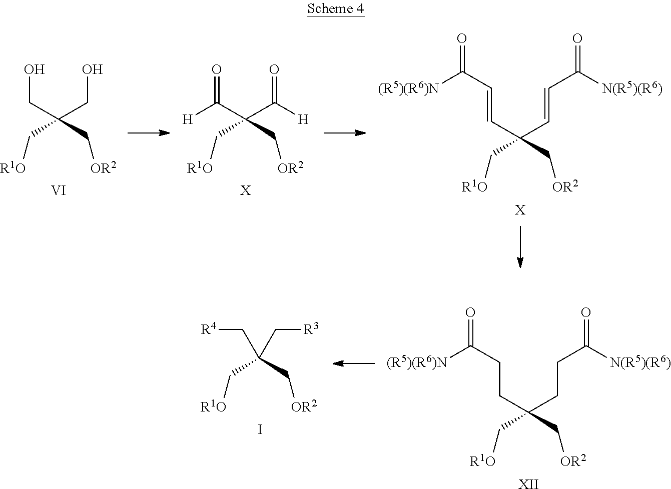

Intermediate VI can be oxidized to intermediate X under well known conditions, including but not limited to Swern oxidation conditions. Intermediate X can be transformed to intermediate XI by reactions known in the art, including but not limited to the Wadsworth-Horner-Emmons reaction. Hydrogenation of intermediate XI under well known conditions provides compound XII, which is reduced, by well known reagents, including but not limited to LiAlH₄, to give compounds of formula I wherein R¹ and R² are alkyl or arylalkyl and R³ and R⁴ are —CH₂CH₂N(R⁵)(R⁶). Acidification or alkylation with an alkylating agent of formula R^{7a}—X, wherein X is a leaving group and R^{7a} is R⁷ as defined herein or a group which may be subsequently transformed to R⁷, provides compounds of formula I wherein R¹ and R² are alkyl or arylalkyl, and R³ and R⁴ are —CH₂CH₂N(R⁵)(R⁶)(R⁷)⁺.

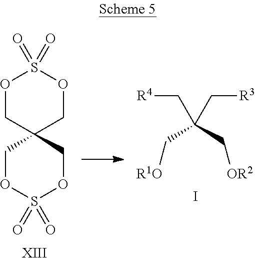

Compounds of formula I, wherein R¹ is as defined herein, R² is R¹, and R³ and R⁴ are —OSO₃⁻, can be prepared by reacting pentaerythritol bicyclic sulfate (XIII), prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386, with an alcohol of formula R¹—OH in the presence of sodium hydride and N,N-dimethylformamide.

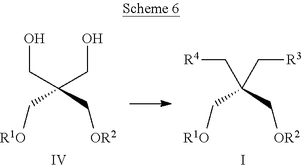

Reaction of intermediate IV, wherein R¹ and R² are alkyl or arylalkyl, with sodium hydride, followed by a (C₂₋₆)alkylene sulfate, provides compounds of formula I wherein R¹ and R² are alkyl or arylalkyl, and R³ and R⁴ are —O(C₂₋₆)alkyl-OSO₃⁻. Alternatively, reaction of intermediate IV, wherein R¹ and R² are alkyl or arylalkyl, with sodium hydride, followed by a (C₂₋₆)sultone, provides compounds of formula I wherein R¹ and R² are alkyl or arylalkyl, and R³ and R⁴ are —O(C₂₋₆)alkyl-SO₃⁻.

### EXAMPLES

Other features of the present invention will become apparent from the following non-limiting examples which illustrate, by way of example, the principles of the invention. It will be apparent to a person of skill in the art that the procedures exemplified below may be used, with appropriate modifications, to prepare other compounds of the invention as described herein. The examples described herein serve to illustrate the utility of the synthetic methods and the materials developed from those methods in the production of compounds according to the invention, and are not meant to be all-inclusive.

N,N-Dimethylformamide is stored over activated molecular sieves for 72 hours, then distilled with reduced pressure over more activated molecular sieves. Methanol is dried with magnesium methoxide. Toluene is dried by reflux over calcium hydride for 10 min followed by distillation from calcium hydride. Sodium hydride is a 60% oil dispersion that is washed with dry hexane under nitrogen before use. Reactions involving sodium hydride are performed in flame-dried glassware. ¹H and ¹³C NMR spectra are recorded at 300 K in 5 mm NMR tubes on a Bruker AC-250 MHz spectrometer operating at 250.13 and 62.9 MHz, respectively or on a Bruker AVANCE-500 NMR operating at 500.13 and 125.08 MHz, respectively, on solutions in chloroform-d, unless otherwise indicated. Chemical shifts are given in parts per million (ppm)(+/−0.01 ppm) relative to that of tetramethylsilane (TMS) (0.00 ppm) in the case of ¹H NMR spectra, and to the central line of chloroform-d (δ=77.16) for ¹³C NMR spectra. All assignments are made with the aid of COSY (COrelation SpectroscopY), HETCOR (HETeronuclear CORrelation), and/or long-range HETCOR experiments at 250 MHz or HSQC (Heteronuclear Single Quantum Correlation) or HMBC (Heteronuclear Multiple Bond Correlation) experiments at 500 MHz. High resolution electrospray mass spectra (HR ESI MS) are re recorded on samples dissolved in methanol using trilysine KKK or rifampicin or the Tuning Mix from Agilent as references. Most thin layer chromatography (TLC) is performed on aluminum-backed plates bearing 200 μm silica gel 60 F₂₅₄ (Merck or Silicycle). Benzylidene acetals are visualized by quenching of fluorescence or by spraying the plate with a solution of 0.2% p-methoxyphenol in ethanol/2NH₂SO₄ (1/1, v/v), as described in Herzner, H.; Eberling, J.; Schultz, M.; Zimmer, J.; Kunz, H. J. Carbohydr. Chem. 1998, 17, 759-776, or an acidic solution of anisaldehyde in ethanol [ethanol (9 mL), anisaldehyde (0.5 mL), and conc. sulfuric acid (0.5 mL), as described in Stahl, E.; Kaltenbach, U. J. Chromatogr. 1961, 5, 351-355], or a solution of 2% ceric sulfate in 1M sulfuric acid, and followed, for all spray reagents, by heating on a hot plate until colour developed. TLC for quaternary ammonium salts is performed on aluminum-backed plates bearing 200 μm basic alumina and developed with the Dragendorff reagent (Thies, H.; Reuther, F. W. Naturwissenschaften 1954, 41, 230-231; Vágújfalvi, D. Planta Med. 1960, 8, 34-43; Stahl, E. Thin Layer Chromatography: A Laboratory Handbook; 2nd ed.; Springer: Heidelberg, 1969, p. 874).

### Example 1A

### 5,5′-Bis(octyloxymethyl)-2-phenyl-1,3-dioxane (1a)

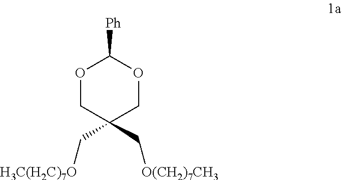

Sodium hydride (60% oil dispersion, washed with hexanes, 8.6 g, 0.22 mol, 2.0 eq) is added in portions slowly to a stirred solution of mono-O-benzylidenepentaerythritol (II, Scheme 1, prepared by the method of Issidorides, C. H.; Gulen, R. C. Organic Syntheses Collected Volume IV, Rabjohn, N., Ed.; John Wiley and Sons: New York, 1963; pp 679-681) (24.11 g, 0.1076 mol) in dry DMF (600 mL) under a nitrogen atmosphere. The stirred reaction mixture is cooled with an ice water bath for one hour, then 1-bromooctane (46.76 mL, 51.90 g, 0.268 mol, 2.5 eq) is added dropwise over 2 h. After the reaction mixture has been stirred 12 h, another addition of sodium hydride (4.5 g, 0.11 mol, 1.0 eq) and 1-bromooctane (20 mL, 0.11 mol, 1.0 eq) is made. If after the reaction mixture has been stirred a further 12 h, TLC shows that some mono-O-octyl product is present, another identical addition is made. When all of the mono-O-octyl derivative has been consumed, the reaction mixture is quenched by the addition of methanol dropwise until foaming ceases. The reaction mixture is filtered under vacuum and the reaction flask and filter are washed with dichloromethane (˜150 mL). The combined filtrate and washings are concentrated and the residue is extracted with hexanes (300 mL, then 200 mL). The combined extracts are washed with water (100 mL), dried (MgSO₄) and concentrated under reduced pressure to an oily residue that is passed through a short silica gel column using hexanes, then 5% ethyl acetate/95% hexanes as eluents. The title compound (1a) is a colourless oil (44.71 g, 93%): R_{F} 0.46 (94:6, hexanes:ethyl acetate); ¹H NMR (500.13 MHz) δ 0.88, 0.89 (2 t, 6H, J=6.5 Hz, 2×Me), 1.20-1.35 (br m, 20H, 10×CH₂), 1.54, 1.57 (2 pentet, 4H, J=6.8 Hz, 2 OCH₂CH₂), 3.22 (s, 2H, eq CCH₂O), 3.35 (t, 2H, J=6.5 Hz, eq octyl OCH₂), 3.45 (t, 2H, J=6.6 Hz, ax octyl OCH₂), 3.71 (s, 2H, ax OCH₂C), 3.88, 4.09 (2d, 4H, J=11.5 Hz, H-4, H-4′, H-6, H-6′), 5.42 (s, 1H, acetal H), 7.31-7.49 (m, 5H, Ph); ¹³C NMR δ 138.5 (q Ph), 128.8 (para Ph), 128.3 (2C, mPh), 126.1 (2C, oPh), 101.7 (acetal C), 71.8 (eq OCH₂CH₂), 71.7 (ax OCH₂CH₂), 70.8 (eq OCH₂C), 70.2 (C-4 and C-6), 69.4 (ax OCH₂C), 38.9 (q C), 2×31.89 (CH₂CH₂CH₃), 29.68, 29.54, 29.51, 29.45, 2×29.34 (6 octyl CH₂), 26.22, 26.19 (CH₂CH₂CH₂O), 2×22.70 (CH₂CH₃), 14.3 (Me); MS ESI: Calc for C₂₈H₄₉O₄ 449.3631. found 449.2; calc for C₂₈H₄₈O₄Na⁺ 471.35. found 471.3; calc for (C₂₈H₆₈O₄)₂Ca²⁺ 468.34. found 468.5; calc for C₂₈H₄₈O₄K⁺ 487.32. found 487.3.

### Example 1B

### 5,5′-Bis(decyloxymethyl)-2-phenyl-1,3-dioxane (1b)

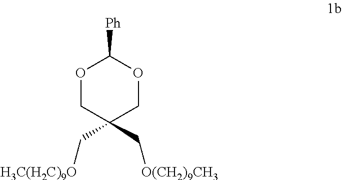

The reaction of mono-O-benzylidenepentaerythritol (II, Scheme 1, prepared by the method of Issidorides, C. H.; Gulen, R. C. Organic Syntheses Collected Volume IV, Rabjohn, N., Ed.; John Wiley and Sons: New York, 1963; pp 679-681) (25.1 g, 0.112 mol), sodium hydride (60% oil dispersion, washed with hexanes, 8.95 g, 0.224 mol, 2.0 eq) and 1-bromodecane (57.9 mL, 61.9 g, 0.280 mol, 2.5 eq) in dry DMF (600 mL) is performed as for Example 1A using additional additions of sodium hydride (1.0 eq) and 1-bromodecane (1.0 eq) as needed. Concentration gives a yellowish oil (50.3 g, 89.1%) that is filtered using a short silica gel column (eluent hexanes). The resulting solution is concentrated to a colourless oil that is crystallized from methanol: mp 29-30° C.; R_{F} 0.48 (94:6 hexanes:ethyl acetate); ¹H NMR δ 0.88 (t, 6H, J=6.4 Hz, 2×Me), 1.20-1.35 (br s, 28H, 14×CH₂), 1.54 (pentet, 4H, J=6.7 Hz, 2 OCH₂CH₂), 3.23 (s, 2H, eq CCH₂O), 3.36 (t, 2H, J=6.4 Hz, eq decyl OCH₂), 3.46 (t, 2H, J=6.6 Hz, ax decyl OCH₂), 3.71 (s, 2H, ax OCH₂C), 3.88, 4.09 (2d, 4H, J=11.7 Hz, H-4, H-4′, H-6, H-6′), 5.42 (s, 1H, acetal H), 7.31-7.48 (m, 5H, Ph); ¹³C NMR δ 138.6 (q Ph), 129.0 (para Ph), 128.4 (2C, mPh), 126.2 (2C, oPh), 101.9 (acetal C), 71.9 (eq OCH₂CH₂), 71.8 (ax OCH₂CH₂), 70.9 (eq OCH₂C), 70.4 (C-4 and C-6), 69.4 (ax OCH₂C), 39.0 (q C), 2×32.06 (CH₂CH₂CH₃), 2×29.81, 29.79, 2×29.76, 29.69, 29.65, 29.62, 2×29.50 (10 decyl CH₂), 2×26.3 (CH₂CH₂CH₂O), 2×22.8 (CH₂CH₃), 14.3 (Me); MS ESI: Calc for C₃₂H₅₇O₄ 505.4. found 505.1. Anal. Calc. for C₃₂H₅₆O₄: C, 76.14; H, 11.18. Found: C, 76.03; H, 10.97.

### Example 1C

### 5,5′-Bis(dodecyloxymethyl)-2-phenyl-1,3-dioxane (1c)

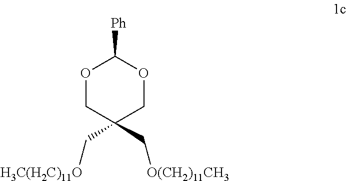

The reaction of mono-O-benzylidenepentaerythritol (II, Scheme 1, prepared by the method of Issidorides, C. H.; Gulen, R. C. Organic Syntheses Collected Volume IV, Rabjohn, N., Ed.; John Wiley and Sons: New York, 1963; pp 679-681) (30.0 g, 0.134 mol), sodium hydride (60% oil dispersion, washed with hexanes, 12 g, 0.30 mol, 2.2 eq) and 1-bromododecane (72 mL, 74.88 g, 0.30 mol, 2.25 eq) in dry DMF (1000 mL) is performed as for Example 1A using additional additions of sodium hydride (1.0 eq) and 1-bromododecane (1.0 eq) as needed. The residue is a solid that is recrystallized from methanol to give colorless needles: yield 57.54 g, 77%; R_{F} 0.51 (94:6 hexanes:ethyl acetate); mp 37.5-38.5° C.; ¹H NMR δ 0.88 (t, 6H, J=6.4 Hz, 2×Me), 1.20-1.35 (br s, 36H, 18×CH₂), 1.54 (pentet, 4H, J=6.3 Hz, 2 OCH₂CH₂), 3.22 (s, 2H, eq CCH₂O), 3.35 (t, 2H, J=6.5 Hz, eq dodecyl OCH₂), 3.46 (t, 2H, J=6.6 Hz, ax dodecyl OCH₂), 3.71 (s, 2H, ax OCH₂C), 3.88, 4.09 (2d, 4H, J=11.7 Hz, H-4, H-4′, H-6, H-6′), 5.41 (s, 1H, acetal H), 7.31-7.48 (m, 5H, Ph); ¹³C NMR δ 138.5 (q Ph), 128.8 (para Ph), 128.2 (2C, mPh), 126.1 (2C, oPh), 101.7 (acetal C), 71.7 (eq CH₂CH₂OC), 71.6 (ax CH₂CH₂OC), 70.7 (eq OCH₂C), 70.2 (C-4 and C-6), 69.3 (ax OCH₂C), 38.9 (q C), 2×31.8 (CH₂CH₂CH₃), 29.70, 29.66, 29.61, 29.53, 29.22 (14 dodecyl CH₂), 26.2 (CH₂CH₂CH₂O), 2×22.7 (CH₂CH₃), 2 ×14.1 (Me); MS ESI: Calc for C₃₆H₆₅O₄ 561.49. found 561.3; calc for C₃₆H₆₄O₄Na⁺583.47. found 583.5; calc for C₃₆H₆₄O₄K⁺599.44. found 599.3.

Anal. Calc. for C₃₆H₆₄O₄: C, 77.09; H, 11.50. Found: C, 77.04, H, 11.92.

### Example 1D

### 2-Phenyl-5,5′-bis(tetradecyloxymethyl)-1,3-dioxane (1d)

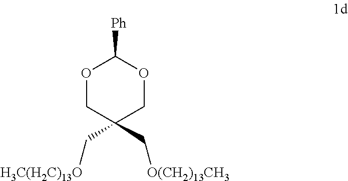

The reaction of mono-O-benzylidenepentaerythritol (II, Scheme 1, prepared by the method of Issidorides, C. H.; Gulen, R. C. Organic Syntheses Collected Volume IV, Rabjohn, N., Ed.; John Wiley and Sons: New York, 1963; pp 679-681) (30.0 g, 0.134 mol), sodium hydride (60% oil dispersion, washed with hexanes, 11.3 g, 0.295 mol, 2.2 eq) and 1-bromotetradecane (84 mL, 78.3 g, 0.282 mol, 2.5 eq) in dry DMF (1000 mL) is performed as for Example 1A using additional additions of sodium hydride (1.0 eq) and 1-bromotetradecane (1.0 eq) as needed to give the title compound (1d) as a solid (73.7 g, 89%): R_{F} 0.53 (94:6 hexanes:ethyl acetate); recrystallized from ethyl acetate; mp 45° C.; ¹H NMR (500 MHz) δ 0.88 (t, 6H, J=6.6 Hz, 2×Me), 1.16-1.37 (br s, 44H, 18×CH₂), 1.55 (br pentet, 4H, J=6.4 Hz, 2 OCH₂CH₂), 3.23 (s, 2H, eq CCH₂O), 3.35 (t, 2H, J=6.4 Hz, eq OCH₂CH₂), 3.46 (t, 2H, J=6.4 Hz, ax OCH₂CH₂), 3.71 (s, 2H, ax OCH₂C), 3.88, 4.09 (2d, 4H, J=11.3 Hz, H-4, H-4′, H-6, H-6′), 5.41 (s, 1H, acetal H), 7.31-7.48 (m, 5H, Ph); ¹³C NMR δ 139.3 (q Ph), 128.9 (para Ph), 128.4 (2C, mPh), 126.2 (2C, oPh), 101.8 (acetal C), 71.9 (eq CH₂CH₂OC), 71.8 (ax CH₂CH₂OC), 70.9 (eq OCH₂C), 70.4 (C-4 and C-6), 69.5 (ax OCH₂C), 39.0 (q C), 2×32.1 (CH₂CH₂CH₃), 2×29.86, 2×29.83, 6×29.81, 2×29.77, 2×29.75, 2×29.66, 2×29.51 (18 tetradecyl CH₂), 2×26.3 (CH₂CH₂CH₂O), 2×22.8 (CH₂CH₃), 2×14.2 (Me); MS ESI: Calc for C₄₀H₇₃O₄ 617.5509. found 617.1; calc for C₄₀H₇₂O₄Na 639.53. found 639.5; calc for C₄₀H₇₂O₄K 655.51. found 655.3.

### Example 2A

### 2,2-Bis(octyloxymethyl)-1,3-propanediol (2a)

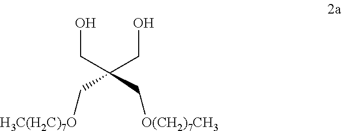

To a solution of compound 1a (Example 1A) (10.02 g, 22.3 mmol) in ethyl acetate (100 mL) is added 10% Pd/C (Degussa type, 0.2 g). The mixture is stirred vigorously under atmospheric pressure H₂(g) for 1 h. More 10% Pd/C (Degussa type, 0.5 g) is added and the solution stirred until uptake of H₂(g) ceases (2 h). The mixture is filtered and the residue washed with dichloromethane (50 mL), then dichloromethane containing 20% methanol (2×50 mL). The filtrate and washings are concentrated to a colourless solid that is recrystallized from methanol: yield 6.89 g, 85%, R_{F} 0.40 (dichloromethane:methanol 96:4); mp 30-32° C. Recrystallization from isopropanol gives colorless crystals: mp 35° C.; ¹H NMR δ 0.88 (t, 6H, J=6.9 Hz, 2×Me), 1.20-1.36 (br m, 20H, 10×CH₂), 1.56 (pentet, 4H, J=6.8 Hz, 2 OCH₂CH₂), 2.82 (t, 2H, J=6.1 Hz, OH), 3.42 (t, 4H, J=6.5 Hz, octyl OCH₂), 3.50 (s, 4H, OCH₂C), 3.65 (d, 4H, CH₂OH); ¹³C NMR δ 73.1 (CCH₂OCH₂CH₂), 72.2 (CH₂CH₂OC), 65.5 (CH₂OH), 44.7 (q C), 31.9 (CH₂CH₂CH₃), 29.50, 29.35 (2 octyl CH₂), 29.64 (OCH₂CH₂), 26.3 (CH₂CH₂CH₂O), 22.8 (CH₂CH₃), 14.2 (Me); MS ESI: Calc for O₂₁H₄₅O₄ 361.33. found 361.1. Anal. Calc. for C₂₁H₄₄O₄: C, 69.95; H, 12.30. Found: C, 69.62; H, 12.68.

### Example 2B

### 2,2-Bis(decyloxymethyl)-1,3-propanediol (2b)

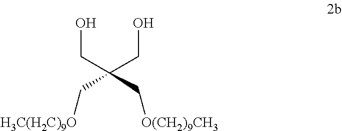

Compound 1b (Example 1B) (10.23 g, 20.3 mmol) is hydrogenated using the procedure of Example 2A in ethyl acetate (100 mL) using 10% Pd/C (Degussa type, 0.2 g) as catalyst to give a colourless solid that is recrystallized from methanol: yield 6.81 g, 80.7%, R_{F} 0.42 (dichloromethane:methanol 96:4); mp 44.5-45° C.; ¹H NMR δ 0.88 (t, 6H, J=6.6 Hz, 2×Me), 1.20-1.35 (br s, 28H, 18×CH₂), 1.55 (pentet, 4H, J=6.5 Hz, 2 OCH₂CH₂), 2.88 (t, 2H, J=6.1 Hz, OH), 3.42 (t, 4H, J=6.5 Hz, dodecyl OCH₂), 3.51 (s, 4H, OCH₂C), 3.65 (d, 4H, CH₂OH); ¹³C NMR δ 73.3 (CCH₂OCH₂CH₂), 72.2 (CH₂CH₂OC), 65.6 (CH₂OH), 44.6 (q C), 32.0 (CH₂CH₂CH₃), 3×29.7, 29.66, 29.56, 29.47 (6 dodecyl CH₂), 26.3 (CH₂CH₂CH₂O), 22.8 (CH₂CH₃), 14.2 (Me); MS ESI: Calc for C₂₅H₅₃O₄ 417.39. found 417.1. Anal. Calc. for C₂₅H₅₂O₄: C, 72.06; H, 12.58. Found: C, 71.98; H, 12.45.

### Example 2C

### 2,2-Bis(dodecyloxymethyl)-1,3-propanediol (2c)

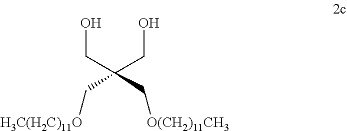

Compound 1c (Example 1C) (10.23 g, 20.3 mmol) is hydrogenated using the procedure of Example 2A in ethyl acetate (100 mL) using 10% Pd/C (Degussa type, 0.2 g) as catalyst to give a colourless solid that is recrystallized from ethyl acetate: yield 6.81 g, 80.7%; R_{F} 0.45 (dichloromethane:methanol 96:4); mp 54-55° C.; ¹H NMR δ 0.88 (t, 6H, J=6.6 Hz, 2×Me), 1.20-1.35 (br s, 36H, 18×CH₂), 1.56 (pentet, 4H, J=6.4 Hz, 2 OCH₂CH₂), 2.69 (br s, 2H, OH), 3.42 (t, 4H, J=6.5 Hz, dodecyl OCH₂), 3.51 (s, 4H, OCH₂C), 3.64 (s, 4H, CH₂OH); ¹³C NMR δ 73.3 (CCH₂OCH₂C), 72.2 (CH₂CH₂OC), 65.6 (CH₂OH), 44.6 (q C), 32.1 (CH₂CH₂CH₃), 29.81, 29.78, 29.77, 29.73, 29.58, 29.50 (6 dodecyl CH₂), 26.3 (CH₂CH₂CH₂O), 29.66 (OCH₂CH₂), 22.8 (CH₂CH₃), 14.3 (Me). MS ESI: Calc for C₂₉H₆₁O₄ 473.46. found 473.3. Anal. Calc. for C₂₉H₆₀O₄: C, 73.67; H, 12.79. Found: C, 73.31; H, 12.68.

### Example 2D

### 2,2-Bis(tetradecyloxymethyl)-1,3-propanediol (2d)

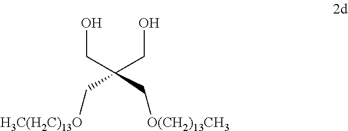

Compound 1d (Example 1D) (10.0 g, 16.2 mmol) is hydrogenated using the procedure of Example 2A in ethyl acetate (200 mL) containing 10% Pd/C (Degussa type, 0.5 g) to give a colourless solid that is recrystallized from ethyl acetate: yield 7.88 g, 92%; R_{F} 0.47 (dichloromethane:methanol 96:4); mp 63-64° C.; ¹H NMR δ 0.88 (t, 6H, J=6.6 Hz, 2×Me), 1.20-1.35 (br s, 44H, 18×CH₂), 1.56 (pentet, 4H, J=6.4 Hz, 2 OCH₂CH₂), 2.83 (br s, 2H, OH), 3.42 (t, 4H, J=6.5 Hz, dodecyl OCH₂), 3.51 (s, 4H, OCH₂C), 3.64 (s, 4H, CH₂OH); ¹³C NMR δ 73.4 (CCH₂OCH₂C), 72.2 (CH₂CH₂OC), 65.7 (CH₂OH), 44.6 (q C), 32.1 (CH₂CH₂CH₃), 29.85, 2×29.83, 29.81, 29.78, 29.75, 29.59, 29.51 (8 tetradecyl CH₂), 29.65 (OCH₂CH₂), 26.3 (CH₂CH₂CH₂O), 22.7 (CH₂CH₃), 14.3 (Me); MS ESI: Calc for C₃₃H₆₉O₄ 529.52. found 529.3. Anal. Calc. for C₃₃H₆₈O₄: C, 74.94; H, 12.96. Found: C, 74.55; H, 13.03.

### Example 3A

### N,N,N′,N′-tetramethyl-5,5-bis(octyloxymethyl)-3,7-dioxa-1,9-nonanediamine (3a)

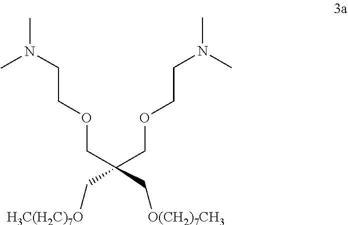

Sodium hydride (15.71 g, 0.393 mol, 10 eq) is added slowly to a stirred solution of 2,2-bis(octyloxymethyl)-1,3-propanediol (2a, Example 2A) (14.15 g, 0.039 mol) in DMF (0.80 L) under nitrogen gas at 50° C. The mixture is then vigorously stirred at 50° C. for 35 min. The reaction mixture is cooled to rt then 2-(dimethylamino)ethyl chloride hydrochloride (22.6 g, 0.157 mol, 4 eq) is added and washed into the stirring mixture with DMF (130 mL). The reaction mixture is stirred at 50° C. under N₂ for 12 h, then quenched with methanol. The mixture is filtered and concentrated and the solid residue is dissolved in dichloromethane. The solution is washed with water (100 mL), dried (MgSO₄) and concentrated to an orange oil: yield 18.0 g, 91%; R_{F} on basic alumina 0.44 (chloroform: ethanol 98:2); ¹H NMR δ 0.88 ppm (t, 6H, J=6.9 Hz, 2×Me), 1.25-1.33 (br m, 20H, 10×CH₂), 1.51 (pentet, 4H, J=6.8 Hz, 2×OCH₂CH₂), 2.26 (s, 12H, 2×N(CH₃)₂), 2.49 (t, 4H, J=5.8 Hz, OCH₂CH₂N), 3.35 (t, 4H, J=6.5 Hz, decyl OCH₂)), 3.36 (s, 4H, OCH₂C), 3.39 (s, 4H, OCH₂C), 3.49 (t, 4H, J=6.0 Hz, OCH₂CH₂N); ¹³C NMR δ 71.9 (CH₂CH₂OC), 70.5 (NCH₂CH₂OCH₂), 70.3 (OCH₂CH₂N), 69.8 (CCH₂OCH₂CH₂C), 58.8 (OCH₂CH₂N), 46.1 (N(CH₃)₂), 45.6 (q C), 32.0 (CH₂CH₂CH₃), 29.70, 29.57, 29.54 (3 octyl CH₂), 29.40 (OCH₂CH₂), 26.3 (CH₂CH₂CH₂O), 22.8 (CH₂CH₃), 14.2 (Me); ESI MS m/z calc for C₂₉H₆₃N₂O₄ (M+1) 503.47. found 503.4; calc for M+Na 425.46. found 525.5.

### Example 3B

### 5,5-bis(decyloxymethyl)-N,N,N′,N′-tetramethyl-3,7-dioxa-1,9-nonanediamine (3b)

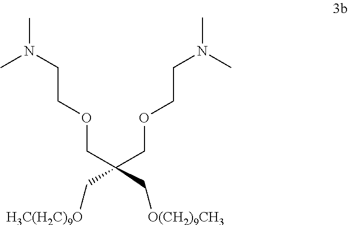

Sodium hydride (10.99 g, 0.183 mol, 10 eq) is added slowly to a stirred solution of 2,2-bis(decyloxymethyl)-1,3-propanediol (2b, Example 2B) (9.5 g, 0.023 mol) in DMF (1 L) under nitrogen gas at 50° C. The mixture is then vigorously stirred at 50° C. for 35 min. The reaction mixture is cooled to rt then 2-(dimethylamino)ethyl chloride hydrochloride (13.2 g, 0.092 mol, 4 eq) is added and washed into the stirring mixture with DMF (130 mL). The reaction mixture is stirred at 50° C. under N₂ for 12 h, then quenched with methanol. The mixture is filtered and concentrated. The solid is dissolved in dichloromethane, filtered and solvent is evaporated off giving an orange oil: yield 12 g, 93%, R_{F} on basic alumina 0.46 (chloroform:ethanol 98:2); ¹H NMR δ 0.88 ppm (t, 6H, J=6.8 Hz, 2×Me), 1.26 (br s, 28H, 14×CH₂), 1.52 (pentet, 4H, J=6.6 Hz, 2 OCH₂CH₂), 2.27 (s, 12H, 2×N(CH₃)₂), 2.51 (t, 4H, J=5.8 Hz, OCH₂CH₂N), 3.35 (t, 4H, J=6.5 Hz, decyl OCH₂), 3.36 (s, 4H, CH₂CH₂CH₂OCH₂C), 3.39 (s, 4H, NCH₂CH₂OCH₂C), 3.51 (t, 4H, J=5.8 Hz, OCH₂CH₂N); ¹³C NMR δ 71.6 (CH₂CH₂OC), 70.4 (NCH₂CH₂OCH₂), 70.1 (OCH₂CH₂N), 69.8 (CCH₂OCH₂CH₂C), 58.7 (OCH₂CH₂N), 46.0 (N(CH₃)₂), 45.5 (q C), 32.1 (CH₂CH₂CH₃), 29.81, 29.79, 29.75, 29.73, 29.65 (5 decyl CH₂), 29.48 (OCH₂CH₂), 26.4 (CH₂CH₂CH₂O), 22.8 (CH₂CH₃), 14.2 (Me); ESI MS m/z calc for C₃₃H₇₁N₂O₄ (M+1): 559.54. Found: 559.5; m/z calc for M+Na, 581.52. found 581.5.

### Example 3C

### 5,5-bis(dodecyloxymethyl)-N,N,N′,N′-tetramethyl-3,7-dioxa-1,9-nonanediamine (3c)

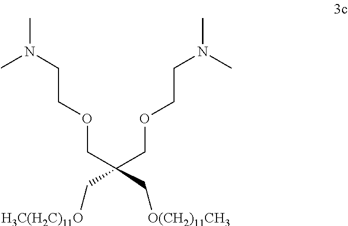

Treatment of compound 2c (Example 2C) (14.04 g, 0.02983 mol) in DMF (1 L) with sodium hydride (26.84 g, 0.4474 mol, 15 eq) and 2-(dimethylamino)ethyl chloride hydrochloride (16.87 g, 0.117 mol, 4 eq) following the procedure of Example 3A gives the title compound (3c) as a orange oil: yield 17.6 g, 98%, R_{F} on basic alumina 0.51 (chloroform:ethanol 98:2); ¹H NMR δ 0.88 ppm (t, 6H, J=6.9 Hz, 2×Me), 1.26-1.29 (br s, 36H, 10×CH₂), 1.52 (pentet, 4H, J=6.6 Hz, 2×OCH₂CH₂), 2.27 (s, 12H, 2×N(CH₃)₂), 2.51 (t, 4H, J=5.9 Hz, OCH₂CH₂N), 3.35 (t, 4H, J=6.5 Hz, dodecyl OCH₂), 3.36 (s, 4H, CH₂CH₂CH₂OCH₂C), 3.39 (s, 4H, NCH₂CH₂OCH₂C), 3.51 (t, 4H, J=5.9 Hz, OCH₂CH₂N); ¹³C NMR δ 71.6 (CH₂CH₂OC), 70.4 (NCH₂CH₂OCH₂C), 70.1 (OCH₂CH₂N), 69.8 (CCH₂OCH₂CH₂C), 58.8 (OCH₂CH₂N), 45.9 (N(CH₃)₂), 45.4 (q C), 32.0 (CH₂CH₂CH₃), 29.82, 29.80, 29.79, 29.78, 29.77, 29.65 (6 decylCH₂), 29.48 (OCH₂CH₂), 26.4 (CH₂CH₂CH₂O), 22.8 (CH₂CH₃), 14.2 (Me); ESI MS m/z calc for C₃₇H₇₉N₂O₄ (M+1) 615.60. Found 615.5. Calc for M+Na, 637.59. found 637.6.

### Example 3D

### N,N,N′,N′-tetramethyl-3,7-dioxa-5,5-bis(tetradecyloxymethyl)-1,9-nonanediamine (3d)

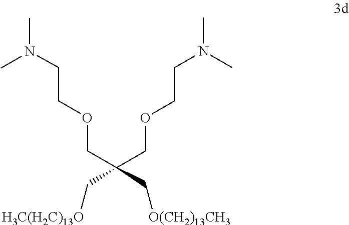

Treatment of compound 2d (Example 2D) (3.2 g, 6.49 mmol) in DMF (300 mL) with sodium hydride (3.894 g, 0.0649 mol, 10 eq) and 2-(dimethylamino)ethyl chloride hydrochloride (3.7 g, 0.026 mol, 4 eq) following the procedure of Example 3A gives the title compound (3d) as a orange oil: yield 3.63 g, 83%, R_{F} 0.04 (methanol); ¹H NMR δ 0.88 ppm (t, 6H, J=6.9 Hz, 2×Me), 1.24-1.32 (br s, 44H, 22×CH₂), 1.52 (pentet, 4H, J=6.8 Hz, 2 OCH₂CH₂), 2.26 (s, 12H, 2×N(CH₃)₂), 2.49 (t, 4H, J=5.9 Hz, OCH₂CH₂N), 3.35 (t, 4H, J=6.5 Hz, tetradecyl OCH₂), 3.36 (s, 4H, CH₂CH₂CH₂OCOCH₂C), 3.39 (s, 4H, NCH₂CH₂OCH₂C), 3.50 (t, 4H, J=5.9 Hz, OCH₂CH₂N); ¹³C NMR δ 71.7 (CH₂CH₂OC), 70.4 (NCH₂CH₂OCH₂C), 70.3 (OCH₂CH₂N), 69.8 (CCH₂OCH₂CH₂C), 58.9 (OCH₂CH₂N), 46.2 (2×N(CH₃)₂), 45.5 (q C), 32.1 (CH₂CH₂CH₃), 3×29.85, 29.83, 29.82, 2×29.80, 29.68 (8 tetradecyl CH₂), 29.51 (OCH₂CH₂), 26.4 (CH₂CH₂CH₂O), 22.8 (CH₂CH₃), 14.3 (Me); ESI MS m/z calc for C₄₁H₈₇N₂O₄ (M+1) 671.67. Found 671.6. Calc for M+Na, 693.65. found 693.7.

### Example 3E

### N,N,N′,N′-Tetramethyl-6,6-bis(octyloxymethyl)-4,8-dioxa-1,11-undecanediamine (3e)

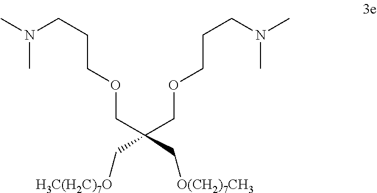

Sodium hydride (1.33 g, 55.4 mmol) is added slowly to a stirred solution of 2,2-dioctyloxymethyl-1,3-propanediol (2a, Example 2A) (2.0 g, 5.5 mmol) in DMF (100 mL) under an N₂ atmosphere at 50° C. The mixture is then stirred at 80° C. for 1 h. The reaction mixture is allowed to cool to rt, 3-chloro-N,N-dimethyl-1-propanamine hydrochloride (1.93 g, 12.2 mmol) is added in portions and the reaction mixture is stirred at 80° C. under nitrogen for another 24 h, then quenched with methanol and filtered. The filtrate is concentrated, and the residue is taken up in ethyl acetate (50 mL). This solution is washed with water (2×20 mL) and brine (20 mL), then dried (Na₂SO₄) and concentrated to a residue that is purified by flash column chromatography. Elution using a gradient of 10 to 15% methanol in dichloromethane gives compound 3e as a light brown liquid: yield 1.6 g (54%); R_{F} on basic alumina 0.6 (dichloromethane:methanol 93:7); ¹H NMR (CDCl₃) δ 0.88 (t, 6H, J=6.5 Hz, 2 CH₃), 1.26-1.35 (m, 20H, 10×CH₂), 1.52 (p, 4H, J=7.0 Hz, 2 OCH₂CH₂), 1.71 (p, 4H, J=6.5 Hz, 2 NCH₂CH₂), 2.22 (s, 12H, 2 N(CH₃)₂), 2.32 (t, 4H, J=7.5 Hz, 2 NCH₂), 3.34 (t, 4H, J=6.5 Hz, 2 CH₂CH₂O), 3.35 (s, 4H, 2 OCH₂), 3.37 (s, 4H, 2 OCH₂), 3.41 (t, 4H, J=6.5 Hz, OCH₂); ¹³C NMR (CDCl₃) δ 71.7 (octyl CH₂O), 70.0 (OCH₂CH₂CH₂N), 69.85 (OCH₂C), 69.78 (OCH₂C), 57.0 (NCH₂), 45.64 (N(CH₃)₂), 45.59 (qC), 32.0 (CH₃CH₂CH₂), 29.82, 29.63, 29.49 (3 octyl CH₂), 28.2 (NCH₂CH₂), 26.4 (OCH₂CH₂CH₂), 22.9 (CH₃CH₂), 14.2 (CH₃); HR ESI MS m/z calcd for C₃₁H₆₇N₂O₄ (M+H) 531.5095. found 531.5087.

### Example 3F

### 6,6-Bis(decyloxymethyl)-N,N,N′,N′-tetramethyl-4,8-dioxa-1,11-undecanediamine (3f)

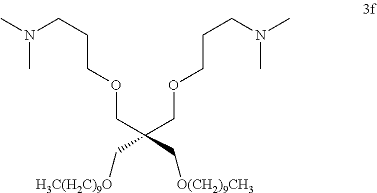

Sodium hydride (0.57 g, 24.0 mmol), 2,2-didecyloxymethyl-1,3-propanediol (2b, Example 2B) (1.0 g, 2.4 mmol) in DMF (100 mL) and 3-chloro-N,N-dimethyl-1-propanamine hydrochloride (1.8 g, 12 mmol) are reacted following the procedure of Example 3E to give compound 3f as a light brown liquid: yield 0.7 g (50%); R_{F} on basic alumina 0.44 (dichloromethane:methanol 95:5); ¹H NMR (CDCl₃) δ 0.88 (t, 6H, J=6.5 Hz, CH₃), 1.28-1.32 (m, 28H, 14×CH₂), 1.52 (p, 4H, J=7.0 Hz, OCH₂CH₂), 1.70 (p, 4H, J=6.5 Hz, NCH₂CH₂), 2.22 (s, 12H, 2 N(CH₃)₂), 2.31 (t, 4H, J=7.5 Hz, NCH₂), 3.34 (t, 4H, J=6.5 Hz, CH₂O), 3.35 (s, 4H, OCH₂), 3.37 (s, 4H, OCH₂), 3.40 (t, 4H, J=6.5 Hz, OCH₂); ¹³C NMR (CDCl₃) δ 71.7 (decyl CH₂O), 70.0 (OCH₂CH₂CH₂N), 69.9 (OCH₂C), 69.8 (OCH₂C), 57.0 (NCH₂), 45.7 (N(CH₃)₂), 45.6 (qC), 32.1 (CH₃CH₂CH₂), 29.85 (×2), 29.79, 29.70, 29.52 (5 decyl CH₂), 28.20 (NCH₂CH₂), 26.4 (OCH₂CH₂CH₂), 22.9 (CH₃CH₂), 14.3 (CH₃); ESI MS m/z calcd for C₃₅H₇₅N₂O₄ (M+H) 587.5712. found 587.5742.

### Example 3G

### 6,6-Bis(dodecyloxymethyl)-N,N,N′,N′-tetramethyl-4,8-dioxa-1,11-undecanediamine (3 g)

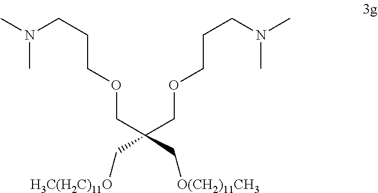

Sodium hydride (1.0 g, 42 mmol), 2,2-didodecyloxymethyl-1,3-propanediol (2c, Example 2C) (2.0 g, 4.2 mmol), and 3-chloro-N,N-dimethyl-1-propanamine hydrochloride (2.60 g, 16.9 mmol) are reacted together following the procedure of Example 3E. The reaction mixture is stirred at 90° C. under N₂ for another 36 h, then quenched with methanol, and filtered and worked up following the procedure of Example 3E to give 3g as a light brown liquid: yield 1.4 g (51%); R_{F} on basic alumina 0.5 (dichloromethane: methanol 97:5); ¹H NMR (CDCl₃) δ 0.88 (t, 6H, J=6.5 Hz, CH₃), 1.24-1.38 (m, 36H, 18×CH₂), 1.52 (pentet, 4H, J=7.0 Hz, OCH₂CH₂), 1.72 (pentet, 4H, J=6.5 Hz, NCH₂CH₂), 2.22 (s, 12H, N(CH₃)₂), 2.32 (t, 4H, J=7.5, NCH₂), 3.35 (t, 4H, J=6.5 Hz, CH₂O), 3.36 (s, 4H, OCH₂), 3.37 (s, 4H, OCH₂), 3.41 (t, 4H, J=6.5 Hz, OCH₂); ¹³C NMR (CDCl₃) δ 71.6 (dodecyl CH₂O), 69.9 (OCH₂CH₂CH₂N), 69.72 (OCH₂C), 69.67 (OCH₂C), 56.9 NCH₂), 45.5 (N(CH₃)₂), 45.5 (qC), 31.9 (CH₃CH₂CH₂), 29.75-29.65 (5C), 29.56, 29.38 (7 dodecyl CH₂), 28.0 (NCH₂CH₂), 26.3 (OCH₂CH₂CH₂), 22.7 (CH₃CH₂), 14.1 (CH₃); HR ESI MS m/z calcd for C₃₉H₈₃N₂O₄ (M+H) 643.6347. found 643.6331.

### Example 3H

### N,N,N′,N′-Tetramethyl-4,8-dioxa-6,6-bis(tetradecyloxymethyl)-1,11-undecanediamine (3h)

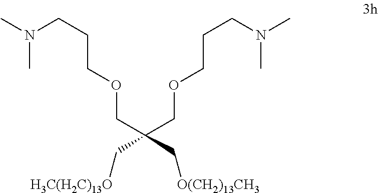

Sodium hydride (2.2 g, 95 mmol), 2,2-tetradecyloxymethyl-1,3-propanediol (2d, Example 2D) (5.0 g, 9.4 mmol), and 3-chloro-N,N-dimethyl-1-propanamine hydrochloride (5.98 g, 37.9 mmol) are reacted and worked up, following the procedure of Example 3E, to give compound 3h as a light brown liquid: yield 2.2 g (35%); R_{F} on basic alumina 0.50 (dichloromethane:methanol 97:5); ¹H NMR (CDCl₃) δ 0.88 (t, 6H, J=6.5 Hz, CH₃), 1.22-1.34 (m, 44H, 22×CH₂), 1.52 (p, 4H, J=6.5 Hz, OCH₂CH₂), 1.71 (p, 4H, J=6.5 Hz, NCH₂CH₂), 2.23 (s, 12H, N(CH₃)₂), 2.33 (t, 4H, J=7.5, NCH₂), 3.34 (t, 4H, J=6.5 Hz, CH₂O), 3.35 (s, 4H, OCH₂), 3.36 (s, 4H, OCH₂), 3.41 (t, 4H, J=6.5 Hz, OCH₂); ¹³C NMR (CDCl₃) δ 71.7 (tetradecyl CH₂O), 70.0 (OCH₂CH₂CH₂N), 69.9 (OCH₂C), 69.8 (OCH₂C), 57.0 (NCH₂), 45.7 N(CH₃)₂, 44.8 (qC), 32.1 (OCH₂CH₂), 29.9 (NCH₂CH₂), 29.72, 29.53, 28.14, 26.4 (decyl CH₂), 22.9 (CH₃CH₂), 14.3 (CH₃); HR ESI MS m/z calcd for C₄₃H₉₁N₂O₄ (M+H) 699.6973. found 699.6986.

### Example 4A

### N,N,N′,N′-tetramethyl-5,5-bis(octyloxymethyl)-3,7-dioxa-1,9-nonanediaminium dichloride (4a)

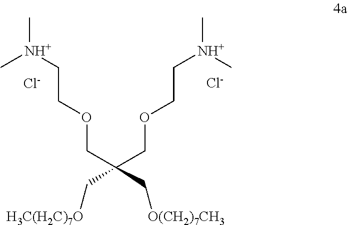

Sodium hydride (3.36 g, 0.14 mol, 10 eq) is added slowly to a stirred solution of 2,2-bis(octyloxymethyl)-1,3-propanediol (2a, Example 2A) (5.05 g, 0.014 mol) in DMF (500 mL) under nitrogen gas at rt and the mixture is stirred vigorously for 1 h. 2-(Dimethylamino)ethyl chloride hydrochloride (8.08 g, 0.05 mol, 4 eq) is added and the reaction mixture is stirred at 50° C. under an N₂ atmosphere for 12 h, then quenched with methanol. The mixture is filtered and concentrated. The residue is taken up in diethyl ether (50 mL) and the resulting solution is washed with brine (50 mL), dried (MgSO₄) and concentrated at 30-35° C. to give N,N,N′,N′-tetramethyl-5,5-bis(octyloxymethyl)-3,7-dioxa-1,9-nonanediamine (3a) as a orange oil. The crude product is taken up in dichloromethane (50 mL) and the resulting solution is shaken with ice cold 2 M HCl (30 mL). The aqueous layer is diluted with brine and this solution is extracted with dichloromethane (5×30 mL). The combined organic layers are dried (MgSO₄) and concentrated to give the title compound (4a) as a light yellow solid, that is crystallized from ethyl acetate and acetone to give colorless crystals: yield 4.90 g, 61%; mp 150-152° C.; R_{F} on basic alumina 0.47 (dichloromethane: ethanol 96:4); ¹H NMR δ 0.88 (t, 6H, J=6.8 Hz, 2×Me), 1.27 (brs, 20H, 10×CH₂), 1.50 (pentet, 4H, J=6.3 Hz, 2 OCH₂CH₂), 2.92 (s, 12H, 2×N(CH₃)₂), 3.33 (t, 4H, J=6.7 Hz, octyl OCH₂), 3.34 (s over broad pattern, 8H, octylOCH₂C and OCH₂CH₂N), 3.51 (s, 4H, N(CH₂)₂OCH₂C), 3.90 (XX′ part of AA′XX′ pattern, 4H, OCH₂CH₂N), 12.0 (br s, 2H, NH); ¹³C NMR δ 71.7 (CH₂CH₂OC), 70.6 (CCH₂O(CH₂)₂N), 69.5 (CCH₂O octyl), 65.8 (OCH₂CH₂N), 56.7 (NCH₂CH₂), 45.1 (q C), 43.6 (N(CH₃)₂), 32.0 (CH₂CH₂CH₃), 29.7, 29.5, 29.3, (octyl CH₂), 26.2 (CH₂CH₂CH₂O), 22.7 (CH₂CH₃), 14.2 (Me); HR ESI MS m/z calcd for C₂₉H₆₃N₂O₄ (M+H) 503.4788. found 503.4784.

### Example 4B

### 5,5-Bis(decyloxymethyl)-N,N,N′,N′-tetramethyl-3,7-dioxa-1,9-nonanediaminium dichloride (4b)

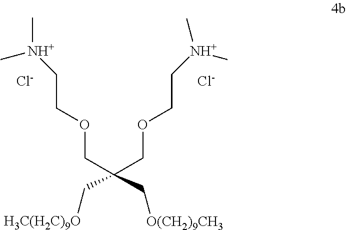

Compound 2b (Example 2B) (5.03 g, 0.012 mol) in DMF (700 mL) with sodium hydride (4.83 g, 0.12 mol, 10 eq) and 2-(dimethylamino)ethyl chloride hydrochloride (6.91 g, 0.05 mol, 4 eq), following the procedure of Example 4A, gives 5,5-bis(decyloxymethyl)-N,N,N′,N′-tetramethyl-3,7-dioxa-1,9-nonanediamine (3b) as a orange oil. Treatment with ice cold 2 M HCl (30 mL), following the procedure of Example 4A, gives the title compound (4b) as a yellow crystalline solid, that is crystallized from ethyl acetate and acetone to give light yellow crystals: yield 5.0 g, 66%; mp 154-155° C.; R_{F} on basic alumina 0.52 (dichloromethane:ethanol 96:4); ¹H NMR δ 0.88 (t, 6H, J=6.9 Hz, 2×Me), 1.26 (brs, 28H, 14×CH₂), 1.50 (pentet, 4H, J=6.4 Hz, 2 OCH₂CH₂), 2.91 (s, 12H, 2×N(CH₃)₂), 3.30 (br AA′ part of AA′XX′ pattern 4H, OCH₂CH₂N), 3.33 (t, 4H, J=6.6 Hz, decyl OCH₂), 3.34 (s, 4H, decylOCH₂C), 3.51 (s, 4H, N(CH₂)₂OCH₂C), 3.90 (XX′ part of AA′XX′ pattern, 4H, OCH₂CH₂N); 12.2 (br s, 2H, NH); ¹³C NMR δ 71.8 (CH₂CH₂OC), 70.7 (CCH₂O(CH₂)₂N), 69.5 (CCH₂Odecyl), 65.8 (OCH₂CH₂N), 56.8 (NCH₂CH₂), 45.2 (q C), 43.7 (N(CH₃)₂), 32.0 (CH₂CH₂CH₃), 29.73, 29.71, 29.68, 29.57, (decyl CH₂), 29.40 (OCH₂CH₂), 26.3 (CH₂CH₂CH₂O), 22.7 (CH₂CH₃), 14.2 (Me), HR ESI MS m/z calcd for C₃₃H₇₁N₂O₄ (M+H) 559.5411. found 559.5411.

### Example 4C

### 5,5-Bis(dodecyloxymethyl)-N,N,N′,N′-tetramethyl-3,7-dioxa-1,9-nonanediaminium dichloride (4c)

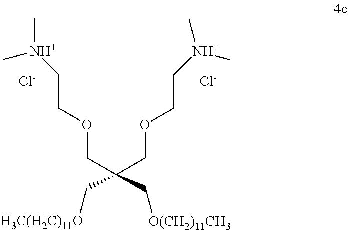

Treatment of compound 2c (Example 2C) (5.040 g, 0.0106 mol) in DMF (700 mL) with sodium hydride (4.26 g, 0.106 mol, 10 eq) and 2-(dimethylamino)ethyl chloride hydrochloride (6.10 g, 0.042 mol, 4 eq), following the procedure of Example 4A, gives 5,5-bis(dodecyloxymethyl)-N,N,N′,N′-tetramethyl-3,7-dioxa-1,9-nonanediamine (3c) as a orange oil, that is taken up in dichloromethane (30 mL). This solution is shaken with ice cold 2 M HCl (30 mL) following the procedure of Example 4A to give a colorless crystalline solid that is recrystallized from ethyl acetate and acetone to give colorless crystals: yield 5.20 g, 71%; mp 145° C.; R_{F} on basic alumina 0.55 (dichloromethane: ethanol 96:4); ¹H NMR δ 0.88 (t, 6H, J=6.8 Hz, 2×Me), 1.26 (br s, 36H, 10×CH₂), 1.50 (pentet, 4H, J=6.3 Hz, 2 OCH₂CH₂), 2.90 (s, 12H, 2×N(CH₃)₂), 3.30 (AA′ part of AA′XX′ pattern, 4H, J_{AX}+J_{A′X}=9.3 Hz OCH₂CH₂N), 3.33 (t, 4H, J=6.5 Hz, dodecyl OCH₂), 3.34 (s, 4H, dodecylOCH₂C), 3.51 (s, 4H, N(CH₂)₂OCH₂C), 3.90 (XX′ part of AA′XX′ pattern, 4H, J_{AX}+J_{A′X}=9.3 Hz OCH₂CH₂N); 12.2 (br s, 2H, NH); ¹³C NMR δ 71.7 (CH₂CH₂OC), 70.6 (CCH₂O(CH₂)₂N), 69.5 (CCH₂Ododecyl), 65.7 (OCH₂CH₂N), 56.7 (NCH₂CH₂), 45.1 (q C), 43.6 (N(CH₃)₂), 31.9 (CH₂CH₂CH₃), 29.7, 29.5, 29.4 (dodecyl CH₂), 26.2 (CH₂CH₂CH₂O), 22.7 (CH₂CH₃), 14.1 (Me), HR ESI MS m/z calcd for C₃₇H₇₉N₂O₄ (M+H) 615.6040. found 615.6046.

### Example 4D

### N,N,N′,N′-Tetramethyl-3,7-dioxa-5,5-bis(tetradecyloxymethyl)-1,9-nonanediaminium dichloride (4d)

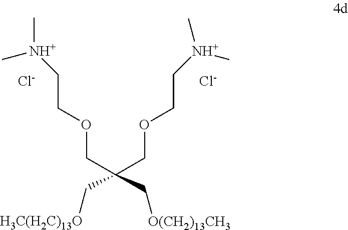

Treatment of compound 2d (Example 2D) (5.28 g, 0.01 mol) in DMF (700 mL) with sodium hydride (4.0 g, 0.10 mol, 10 eq) and 2-(dimethylamino)ethyl chloride hydrochloride (5.7 g, 0.040 mol, 4 eq), following the procedure of Example 4A, gives N,N,N′,N′-tetramethyl-3,7-dioxa-5,5-bis(tetradecyloxymethyl)-1,9-nonanediamine (3d) as a orange oil. Treatment with ice cold 2 M HCl (30 mL), following the procedure of Example 4A, gives the title compound (4d) as a colorless crystalline solid, that is recrystallized from ethyl acetate and acetone to give colorless crystals: yield 5.55 g, 75%; mp 148-150° C.; R_{F} on basic alumina 0.57 (dichloromethane:ethanol 96:4); ¹H NMR δ 0.88 (t, 6H, J=6.9 Hz, 2×Me), 1.26 (br s, 44H, 22×CH₂), 1.50 (pentet, 4H, J=6.4 Hz, 2 OCH₂CH₂), 2.93 (s, 12H, 2×N(CH₃)₂), 3.30 (br AA′ part of AA′XX′ pattern, 4H, OCH₂CH₂N), 3.33 (t, 4H, J=6.5 Hz, tetradecyl OCH₂), 3.34 (s, 4H, tetradecylOCH₂C), 3.51 (s, 4H, N(CH₂)₂OCH₂C), 3.90 (XX′ part of AA′XX′ pattern, 4H, OCH₂CH₂N); 12.0 (br s, 2H, NH); ¹³C NMR δ 71.7 (CH₂CH₂OC), 70.6 (CCH₂O(CH₂)₂N), 69.4 (CCH₂O tetradecyl), 65.7 (OCH₂CH₂N), 56.7 (NCH₂CH₂), 45.1 (q C), 43.6 (N(CH₃)₂), 31.9 (CH₂CH₂CH₃), 29.7, 29.7, 29.5, (tetradecyl CH₂), 26.2 (CH₂CH₂CH₂O), 22.7 (CH₂CH₃), 14.1 (Me); HR ESI MS m/z calcd for C₄₁H₂O₄ (M+H) 671.6666. found 671.6662.

### Example 4E

### N,N,N′,N′-Tetraethyl-5,5-bis(octyloxymethyl)-3,7-dioxa-1,9-nonanediaminium dihydrochloride (4e)

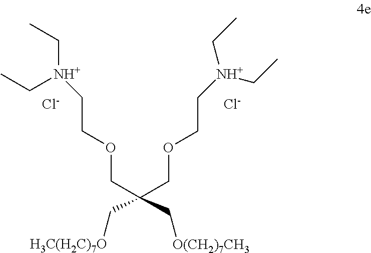

Sodium hydride (6.10 g, 0.15 mol, 10 eq) is added slowly to a stirred solution of 2,2-dioctyloxymethyl-1,3-propanediol (2a, Example 2A) (5.50 g, 0.015 mol) in THF (500 mL) under N₂ gas at rt. When foaming ceases, the mixture is stirred vigorously at 60° C. for 1 h. The reaction mixture is cooled to rt, then 2-bromo-N,N-diethylethylamine hydrobromide (15.95 g, 0.061 mol, 4.0 eq) is added. The reaction mixture is stirred at 60° C. under N₂ gas for 12 h, then quenched with methanol. The reaction mixture is filtered and the filtrate concentrated to a syrupy residue. The residue is taken up in diethyl ether (50 mL) and the resulting solution is washed with brine (50 mL), dried (MgSO₄) and concentrated at 30-35° C. to give crude N,N,N′,N′-tetraethyl-5,5-bis(octyloxymethyl)-3,7-dioxa-1,9-nonanediamine. The crude product is taken up in dichloromethane (50 mL) and the resulting solution is shaken with ice cold 2 M HCl (30 mL). The aqueous layer is diluted with brine (20 mL) and this solution is extracted with dichloromethane (5×30 mL). The combined organic layers are dried (MgSO₄) and concentrated to give the title compound 4e as a colorless solid, that is crystallized from ethyl acetate and acetone to give colorless granules: yield 7.20 g, 75%; mp 155° C.; R_{F} 0.5 (96:4 dichloromethane:ethanol); ¹H NMR δ 0.88 (t, 6H, J=6.8 Hz, 2×Me), 1.27 (brs, 20H, 10×CH₂), 1.42 (t, 12H, J=7.2 Hz, 4×Me), 1.51 (pentet, 4H, J=6.3 Hz, 2 OCH₂CH₂), 3.21 (very br AB part of ABX₃ pattern, 8H, NCH₂CH₃), 3.26 (br t, 4H, NCH₂CH₂O), 3.31 (s, 4H, octyl OCH₂C), 3.33 (t, 4H, J=6.6 Hz, octyl OCH₂), 3.44 (s, 4H, N(CH₂)₂OCH₂C), 3.91 (t, 4H, J=4.3 Hz, OCH₂CH₂N); 12.0 (br s, 2H, NH); ¹³C NMR δ 71.7 (CH₂CH₂OC), 70.6 (CCH₂O(CH₂)₂N), 69.4 (CCH₂O octyl), 65.8 (OCH₂CH₂N), 50.9 (NCH₂CH₂), 47.3 (NCH₂CH₃), 45.1 (q C), 32.0 (CH₂CH₂CH₃), 29.6, 29.5, 29.3 (octyl CH₂), 26.2 (CH₂CH₂CH₂O), 22.7 (CH₂CH₃), 14.1 (Me), 8.8 (Me); HR ESI MS m/z calcd for C₃₃H₇₂N₂O₄/2 (M+2H/2) 280.2741. found 280.2741.

### Example 4F

### 5,5-Bis(decyloxymethyl)-N,N,N′,N′-tetraethyl-3,7-dioxa-1,9-nonanediaminium dihydrochloride (4f)

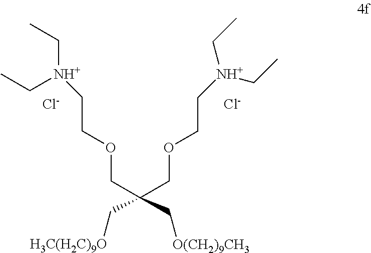

Sodium hydride (0.96 g, 0.024 mol, 10 eq) is added slowly to a stirred solution of 2,2-didecyloxymethyl-1,3-propanediol (2b, Example 2B) (1.0 g, 0.0024 mol) in THF (50 mL) under nitrogen gas at room temperature. When foaming ceases, the mixture is stirred vigorously at 50° C. for 1 h. The reaction mixture is cooled to rt, then 2-bromo-N,N-diethylethylamine hydrobromide (2.5 g, 0.0096 mol, 4 eq) is added. The reaction mixture is stirred at 60° C. under nitrogen gas for 12-18 h, then quenched with methanol. The reaction mixture is filtered and the filtrate concentrated to a syrupy residue. The residue is taken up in diethyl ether (15 mL) and the resulting solution is washed with water (3×5 mL), dried (MgSO₄) and concentrated at 30-35° C. to give crude 1,3-bis[2-(N,N-diethylamino)ethoxy]-2,2-bis(decyloxymethyl)propane. The crude product is taken up in dichloromethane (10 mL) and the resulting solution is shaken with ice cold 2 M HCl (10 mL). The aqueous layer is diluted with brine (5 mL) and this solution is extracted with dichloromethane (5×10 mL). The combined organic layers are dried (MgSO₄) and concentrated to give the title compound (40 as a colourless solid, yield 0.92 g, that is crystallized from ethyl acetate and acetone to give colourless granules: yield 0.84 g, 51%; mp 144° C.; R_{F} 0.63 (96:4 dichloromethane:ethanol); ¹H NMR δ 0.88 ppm (t, 6H, J=6.9 Hz, 2×Me), 1.26 (brs, 28H, 14×CH₂), 1.42 (t, 12H, J=7.3 Hz, 4×Me), 1.51 (pentet, 4H, J=6.4 Hz, 2 OCH₂CH₂), 3.21 (very br AB part of ABX₃ pattern, 8H, NCH₂CH₃), 3.26 (br t, 4H, NCH₂CH₂O), 3.30 (s, 4H, decylOCH₂C), 3.33 (t, 4H, J=6.6 Hz, decyl OCH₂), 3.44 (s, 4H, N(CH₂)₂OCH₂C), 3.92 (t, 4H, J=4.6 Hz, OCH₂CH₂N); 12.05 (br s, 2H, NH); ¹³C NMR δ 71.8 (CH₂CH₂OC), 70.7 (CCH₂O(CH₂)₂N), 69.5 (CCH₂Odecyl), 65.9 (OCH₂CH₂N), 51.1 (NCH₂CH₂), 47.4 (NCH₂CH₃), 45.2 (q C), 32.0 (CH₂CH₂CH₃), 29.73, 29.70, 29.57 (decyl CH₂), 29.40 (OCH₂CH₂), 26.3 (CH₂CH₂CH₂O), 22.7 (CH₂CH₃), 14.2 (Me), 8.8 (Me); HR ESI MS m/z calc for C₃₇H₇₉N₂O₄ (M+H) 615.6040. found 615.6036.

### Example 4G

### 5,5-Bis(dodecyloxymethyl)-N,N,N′,N′-tetraethyl-3,7-dioxa-1,9-nonanediaminium dihydrochloride (4g)

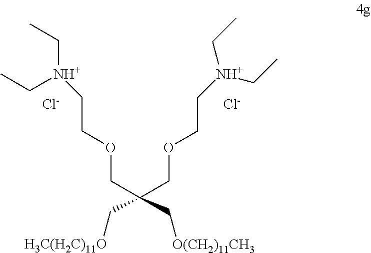

Treatment of compound 2c (Example 2C) (9.38 g, 0.0198 mol) in THF (500 mL) with sodium hydride (7.90 g, 0.198 mol, 10 eq) and 2-bromo-N,N-diethylethylamine hydrobromide (20.7 g, 0.079 mol, 4 eq), following the procedure of Example 4E, gives a crude product, 1,3-bis[2-(N,N-diethylamino)ethoxy]-2,2-bis(dodecyloxymethyl)propane. This compound is taken up in dichloromethane (30 mL) and the resulting solution is shaken with ice cold 2 M HCl (30 mL). The aqueous layer is diluted with brine (30 mL) and this solution is extracted with dichloromethane (5×30 mL), following the procedure of Example 4E, to give the title compound 4g as a colourless solid that is crystallized from ethyl acetate and acetone to give colourless granules: yield 10.4 g, 70.9%; mp 150° C.; R_{F} 0.65 (96:4 dichloromethane:ethanol); ¹H NMR δ 0.88 ppm (t, 6H, J=6.9 Hz, 2×Me), 1.26 (brs, 36H, 18×CH₂), 1.42 (t, 12H, J=7.3 Hz, 4×Me), 1.51 (pentet, 4H, J=6.4 Hz, 2 OCH₂CH₂), 3.21 (very br AB part of ABX₃ pattern, 8H, NCH₂CH₃), 3.26 (br t, 4H, J=4.4 Hz, NCH₂CH₂), 3.30 (s, 4H, dodecylOCH₂C), 3.33 (t, 4H, J=6.6 Hz, dodecyl OCH₂), 3.44 (s, 4H, N(CH₂)₂OCH₂C), 3.92 (t, 4H, J=4.6 Hz, OCH₂CH₂); 12.05 (br s, 2H, NH); ¹³C NMR δ 71.9 (CH₂CH₂OC), 70.8 (CCH₂O(CH₂)₂N), 69.5 (CCH₂Ododecyl), 66.9 (OCH₂CH₂N), 51.1 (NCH₂CH₂), 47.4 (NCH₂CH₃), 45.2 (q C), 32.0 (CH₂CH₂CH₃), 29.79, 29.63, 29.46 (dodecyl CH₂), 26.3 (CH₂CH₂CH₂O), 22.9 (CH₂CH₃), 14.2 (Me), 8.8 (Me); HR ESI MS m/z calc for C₄₁H₈₇N₂O₄(M+H) 671.6666. found 671.6669.

### Example 4H

### N,N,N′,N′-Tetraethyl-3,7-dioxa-5,5-bis(tetradecyloxymethyl)-1,9-nonanediaminium dihydrochloride (4h)

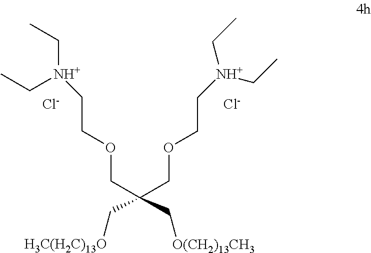

Treatment of compound 2d (Example 2D) (10.0 g, 0.0189 mol) in THF (500 mL) with sodium hydride (7.57 g, 0.189 mol, 10 eq) and 2-bromo-N,N-diethylethylamine hydrobromide (19.7 g, 0.076 mol, 4 eq), following the procedure of Example 4E, gives a crude light yellow syrup, 1,3-bis[2-(N,N-diethylammonio)ethoxy]-2,2-bis(tetradecyloxymethyl)propane. The syrup is taken up in dichloromethane (50 mL) and the solution is shaken with ice cold 2 M HCl (50 mL) following the procedure of Example 4E, to give a colourless solid that is crystallized from ethyl acetate and acetone to give colourless granules: yield 11.5 g, 76.2%; mp 146° C.; R_{F} 0.69 (96:4 dichloromethane:ethanol); ¹H NMR δ 0.88 ppm (t, 6H, J=6.9 Hz, 2×Me), 1.26 (brs, 44H, 22×CH₂), 1.41 (t, 12H, J=7.2 Hz, 4×Me), 1.51 (pentet, 4H, J=6.4 Hz, 2 OCH₂CH₂), 3.20 (very br AB part of ABX₃ pattern, 8H, NCH₂CH₃), 3.24 (br t, NCH₂CH₂), 3.30 (s, 4H, tetradecylOCH₂C), 3.33 (t, 4H, J=6.6 Hz, tetradecyl OCH₂), 3.44 (s, 4H, N(CH₂)₂OCH₂C), 3.91 (t, 4H, J=4.5 Hz, OCH₂CH₂N), 12.05 (brs, 2H, NH); ¹³C NMR δ 71.8 (CH₂CH₂OC), 70.8 (CCH₂O(CH₂)₂N), 69.5 (CCH₂Otetradecyl), 66.1 (OCH₂CH₂N), 51.2 (NCH₂CH₂), 47.2 (NCH₂CH₃), 45.2 (q C), 32.0 (CH₂CH₂CH₃), 29.72, 29.77, 29.65, 29.47 (tetradecyl CH₂), 26.4 (CH₂CH₂CH₂O), 22.8 (CH₂CH₃), 14.2 (Me), 8.9 (Me); HR ESI MS m/z calc for C₄₅H₉₅N₂O₄ (M+H) 727.7292. found 727.7293.

### Example 5A

### 1,3-Bis[2-(N,N,N-trimethylammonio)ethoxy]-2,2-bis(octyloxymethyl)propane diiodide (5a)

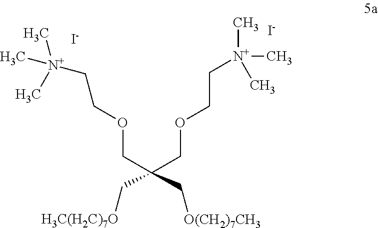

Compound 3a (Example 3A) (7.49 g, 0.0149 mol) is shaken with methyl iodide (8.28 g, 0.0584 mol, 4 eq) for 2 min, then dichloromethane (100 mL) is added and shaking is continued for 5 min. The reaction mixture is concentrated to a yellow solid that is washed with acetone, then crystallized from toluene:ethanol 10:1 to give colourless crystals: yield 10.17 g, 86.8%; recrystallized from ethyl acetate: methanol; R_{F} on basic alumina 0.54 (butanol, water, methanol 4:1:trace); mp softens 180° C., melts 188-189° C.; ¹H NMR δ 0.89 ppm (t, 6H, J=6.9 Hz, 2×Me), 1.28-1.32 (brs, 20H, 10×CH₂), 1.51 (pentet, 4H, J=6.3 Hz, 2 OCH₂CH₂), 3.30 (s, 4H, OCH₂C), 3.33 (t, 4H, J=6.6 Hz, octyl OCH₂), 3.51 (s, 4H OCH₂C), 3.54 (s, 18H, 2×N(CH₃)₃), 3.94 (br s, 4H, OCH₂), 4.02 (br m, 4H, CH₂N); ¹³C NMR δ 71.8 (CH₂CH₂OC), 70.9 (CCH₂OCH₂C), 69.4, (CCH₂OCH₂C), 65.8 (CH₂N), 65.4 (OCH₂CH₂N), 54.9 (N(CH₃)₃), 45.2 (q C), 31.9 (CH₂CH₂CH₃), 29.66, 29.41, 29.32 (3 octyl CH₂), 29.46 (OCH₂CH₂), 26.2 (CH₂CH₂CH₂O), 22.7 (CH₂CH₃), 14.2 (Me); ESI MS m/z calc for C₃₁H₆₈N₂O₄I: 659.45. found 659.1 (M−I). Anal. Calc for C₃₁H₆₈N₂O₄I₂: C, 47.33; H, 8.71; N, 3.56. Found: C, 47.34; H, 8.51; N, 3.29.

### Example 5B

### 1,3-Bis[2-(N,N,N-trimethylammonio)ethoxy]-2,2-bis(decyloxymethyl)propane diiodide (5b)

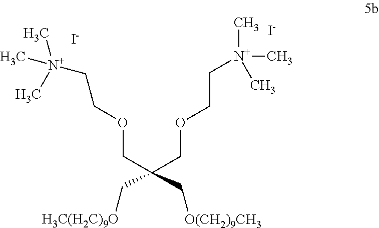

Compound 3b (Example 3B) (11.0 g, 0.0197 mol) is shaken with methyl iodide (4.91 mL, 11.2 g, 0.0716 mol, 4 eq) for 2 min, then dichloromethane (250 mL) is added and shaking is continued for 5 min. The reaction mixture is concentrated to a solid that is crystallized from toluene:ethanol 10:1 to give colorless crystals: yield 16 g, 96%, that are recrystallized from ethyl acetate-methanol to give colorless translucent cubes: R_{F} on basic alumina 0.59 (butanol, water, methanol 4:1: trace); mp 100-110° C. becomes opaque, 180-183° C., clears, 186° C., melts; ¹H NMR δ 0.88 ppm (t, 6H, J=6.9 Hz, 2×Me), 1.27-1.31 (brs, 28H, 14×CH₂), 1.50 (pentet, 4H, J=6.3 Hz, 2 OCH₂CH₂), 3.29 (s, 4H, OCH₂C), 3.33 (t, 4H, J=6.6 Hz, decyl OCH₂), 3.51 (s, 4H OCH₂C), 3.53 (s, 18H, 2×N(CH₃)₃), 3.94 (br s, 4H, OCH₂), 4.02 (br m, 4H, CH₂N); ¹³C NMR δ 71.8 (CH₂CH₂OC), 70.9 (CCH₂OCH₂C), 69.4 (CCH₂OCH₂C), 65.8 (CH₂N(CH₃)₃), 65.4 (OCH₂CH₂N), 54.9 (N(CH₃)₃), 45.1 (q C), 31.9 (CH₂CH₂CH₃), 29.65, 29.63, 29.60, 29.41 (5 decyl CH₂), 29.49 (OCH₂CH₂), 26.2 (CH₂CH₂CH₂O), 22.7 (CH₂CH₃), 14.1 (Me); ESI MS m/z calc for C₃₅H₇₇N₂O₄I: 715.52. found 715.3 (M−I). Anal. Calc for C₃₅H₇₆N₂O₄I₂, 49.88; H, 9.09, N, 3.32. Found: C, 49.84; H, 9.16; N, 3.14.

### Example 5C

### 1,3-Bis[2-(N,N,N-trimethylammonio)ethoxy]-2,2-bis(dodecyloxymethyl)propane diiodide (5c)

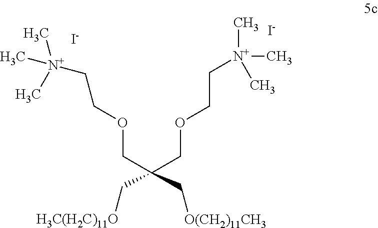

Treatment of compound 3c (Example 3C) (17.6 g, 0.0286 mol) with methyl iodide (7.14 mL, 16.2 g, 0.114 mol, 4 eq) following the procedure of Example 5A gives the title compound (5c) as colorless crystals: yield 21.44 g, 84.8%; recrystallized from ethyl acetate—methanol to give colorless needles: R_{F} on basic alumina 0.62 (butanol, water, methanol 4:1:trace); mp 100-115° C. becomes opaque, 155-180° C., clears, 183° C., melts; ¹H NMR δ 0.88 ppm (t, 6H, J=6.9 Hz, 2×Me), 1.26-1.31 (br s, 36H, 18×CH₂), 1.50 (pentet, 4H, J=6.3 Hz, 2 OCH₂CH₂), 3.29 (s, 4H, OCH₂C), 3.33 (t, 4H, J=6.6 Hz, dodecyl OCH₂), 3.53 (s, 18H, 2×N(CH₃)₃), 3.51 (s, 4H, OCH₂C), 3.94 (br s, 4H, OCH₂), 4.02 (br m, 4H, 2×CH₂N(CH₃)₃); ¹³C NMR δ 71.8 (CH₂CH₂OC), 70.9 (CH₂OCH₂C), 69.4 (CCH₂OCH₂C), 65.9 (CH₂N), 65.4 (OCH₂CH₂N), 55.0 (N(CH₃)₃), 45.2 (q C), 32.0 (CH₂CH₂CH₃), 29.75, 29.75, 29.72, 29.41 (7 dodecyl CH₂), 29.58 (OCH₂CH₂), 26.3 (CH₂CH₂CH₂O), 22.8 (CH₂CH₃), 14.2 (Me); ESI MS Calc for C₃₉H₈₄N₂O₄I 771.58. found 771.3 (M−I). Anal. Calc. for C₃₉H₈₄N₂O₄I₂: C, 52.11; H, 9.42; N, 3.12. Found: C, 52.11; H, 9.24; N, 2.99.

### Example 5D

### 1,3-Bis[2-(N,N,N-trimethylammonio)ethoxy]-2,2-bis(tetradecyloxymethyl)propane diiodide (5d)

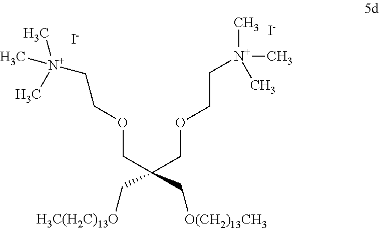

Treatment of compound 3d (Example 3D) (3.63 g, 0.00570 mol) with methyl iodide (1.42 mL, 3.236 g, 0.0228 mol, 4 eq) following the procedure of Example 5A gives the title compound (5d) as colorless crystals: yield 3.7 g, 69%; recrystallized from ethyl acetate-methanol as opaque colorless crystals; R_{F} on basic alumina 0.65 (butanol, water, methanol 4:1:trace); mp, 160-180° C., becomes transparent, 185° C., melts; ¹H NMR δ 0.88 ppm (t, 6H, J=6.9 Hz, 2×Me), 1.26-1.31 (brs, 44H, 22×CH₂), 1.50 (pentet, 4H, J=6.3 Hz, 2 OCH₂CH₂), 3.29 (s, 4H, 2×OCH₂C), 3.33 (t, 4H, J=6.6 Hz, tetradecyl OCH₂), 3.52 (s, 4H, 2×OCH₂C), 3.53 (s, 18H, 2×N(CH₃)₃), 3.93 (br s, 4H, 2×OCH₂CH₂N), 4.02 (br m, 4H, 2×CH₂N); ¹³C NMR δ 71.9 (CH₂CH₂OC), 70.9 (CCH₂OCH₂C), 69.4 (CCH₂OCH₂C), 65.8 (CH₂N), 65.5 (OCH₂CH₂N), 54.9 (N(CH₃)₃), 45.2 (q C), 32.0 (CH₂CH₂CH₃), 29.81, 29.75, 29.64, 29.41 (9 tetradecyl CH₂), 29.45 (OCH₂CH₂), 26.3 (CH₂CH₂CH₂O), 22.8 (CH₂CH₃), 14.2 (Me); ESI MS m/z calc for C₄₃H₉₁N₂O₄I: 827.59. found 827.3 (M−I). Anal. Calc for C₄₃H₉₂N₂O₄I₂: C, 54.08; H, 9.71; N, 2.93. Found: C, 54.16; H, 9.53; N, 2.83.

### Example 5E

### N,N,N,N′,N′,N′-Hexaethyl-5,5-bis(octyloxymethyl)-3,7-dioxa-1,9-nonanediammonium dibromide (5e)

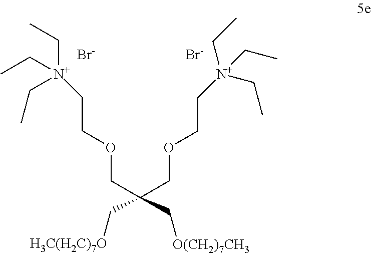

Salt 4e (Example 4E) (1.44 g, 2.28 mmol) is dissolved in a NaOH solution (2 M, 15 mL). The resulting mixture is extracted with diethyl ether (3×5 mL) to yield a colorless syrup of N,N,N′,N′-tetraethyl-5,5-bis(octyloxymethyl)-3,7-dioxa-1,9-nonanediamine, yield 1.08 g, 85%; R_{F} 0.43 on basic alumina (dichloromethane:ethanol, 98:2); ¹H NMR δ 0.88 (t, 6H, J=6.8 Hz, 2×Me), 1.03 (t, J=7.2 Hz, 12H, 4×Me) 1.28 (br s, 20H, 10×CH₂), 1.51 (pentet, 4H, J=6.8 Hz, 2 OCH₂CH₂), 2.57 (q, 8H, J=7.1 Hz, 2×N(CH₂CH₃)₂), 2.65 (t, 4H, J=6.2 Hz, 2NCH₂CH₂), 3.35 (t, J=6.5 Hz, 4H, octyl OCH₂), 3.35 (s, 4H, decylOCH₂C), 3.38 (s, 4H, CCH₂O(CH₂)₂N), 3.47 (t, J=6.3 Hz, 4H, 2 OCH₂CH₂); ¹³C NMR δ 71.6 (CH₂CH₂OC), 70.4 (CCH₂O(CH₂)₂N), 70.4 (CH₂CH₂N), 69.8 (CCH₂O octyl), 52.1 (NCH₂CH₂), 47.8 (NCH₂CH₃), 45.4 (q C), 32.0 (CH₂CH₂CH₃), 29.8, 29.6, 29.5, 29.3 (octyl CH₂), 26.3 (CH₂CH₂CH₂O), 22.8 (CH₂CH₃), 14.2 (Me), 12.1 (CH₂CH₃).

Ethyl bromide (98%, 2.78 mL, 37.3 mmol, 20.0 eq) is added to a stirred solution of N,N,N′,N′-tetraethyl-5,5-bis(octyloxymethyl)-3,7-dioxa-1,9-nonanediamine (1.04 g, 1.86 mmol) in a mixture of THF and ethanol (6 mL) (2:1). Potassium carbonate (0.5 g, 3.7 mmol, 2 eq) is added and the resulting mixture is refluxed for 26 h, then cooled to rt, filtered, and the filtrate is concentrated to give the title compound (5e) as a colorless sticky solid (1.92 g). The salt precipitated from ethyl acetate containing a few drops of methanol, yield 1.13 g, 78%; mp 157° C.; R_{F} 0.53 on basic alumina (butanol:water:methanol 20:5:2); ¹H NMR δ 0.88 (t, 6H, J=6.9 Hz, 2×Me), 1.28 (br s, 20H, 10×CH₂), 1.41 (t, J=7.2 Hz, 12H, 6×Me), 1.51 (pentet, 4H, J=6.5 Hz, 2 OCH₂CH₂), 3.28 (s, 4H, octylOCH₂C), 3.33 (t, J=6.6 Hz, 4H, octyl OCH₂), 3.47 (s, 4H, CCH₂O(CH₂)₂N), 3.4 7 (q, 12H, J=7.2 Hz, 2 N(CH₂CH₃)₂), 3.79 (AA′ part of AA′BB′ pattern, 4H, 2 NCH₂CH₂), 3.94 (BB′ part of AA′BB′ pattern, 4H, 2OCH₂CH₂); ¹³C NMR δ 71.9 (CH₂CH₂OC), 71.1 (CCH₂O(CH₂)₂N), 69.5 (CCH₂O octyl), 65.1 (OCH₂CH₂N), 57.8 (NCH₂CH₂), 54.4 (NCH₂CH₃), 45.3 (q C), 31.9 (CH₂CH₂CH₃), 29.7, 29.6, 29.4, (octyl CH₂), 26.3 (CH₂CH₂CH₂O), 22.7 (CH₂CH₃), 14.2 (Me), 8.4 (CH₂CH₃); HR ESI MS m/z calcd for C₃₇H₈₀N₂O₄/2 (M−2Br)/2 308.3054. found 308.3038.

### Example 5F

### 5,5-Bis(decyloxymethyl)-N,N,N,N′,N′,N′-hexaethyl-3,7-dioxa-1,9-nonanediammonium dibromide (5f)

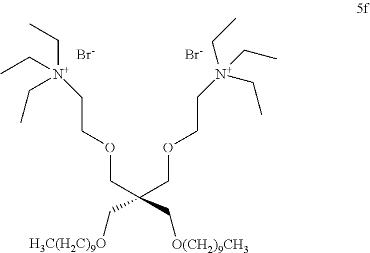

Salt 4f (Example 4F) (0.87 g, 1.18 mmol) is dissolved in a NaOH solution (2 M, 10 mL). The resulting mixture is extracted with dichloromethane (3×5 mL) to yield a colourless syrup of 5,5-bis(decyloxymethyl)-N,N,N′,N′-tetraethyl-3,7-dioxa-1,9-nonanediamine, yield 0.69 g, 95.8%; R_{F} 0.44 on basic alumina (dichloromethane ethanol, 98:2); ¹H NMR δ 0.88 ppm (t, 6H, J=6.9 Hz, 2×Me), 1.03 (t, J=7.2 Hz, 12H, 4×Me) 1.26 (br s, 28H, 14×CH₂), 1.52 (pentet, 4H, J=6.7 Hz, 2 OCH₂CH₂), 2.57 (q, 8H, J=7.14 Hz, 2×N(CH₂CH₃)₂), 2.65 (t, 4H, J=6.2 Hz, 2NCH₂CH₂), 3.35 (t, J=6.5 Hz, 4H, decyl OCH₂), 3.35 (s, 4H, decylOCH₂C), 3.38 (s, 4H, CCH₂O(CH₂)₂N), 3.47 (t, J=6.2 Hz, 4H, 2 OCH₂CH₂); ¹³C NMR δ 71.7 (CH₂CH₂OC), 70.4 (CCH₂O(CH₂)₂N), 70.4 (CH₂CH₂N), 69.8 (CCH₂Odecyl), 52.2 (NCH₂CH₂), 47.6 (NCH₂CH₃), 45.3 (q C), 32.0 (CH₂CH₂CH₃), 29.7, 29.6, 29.5, 29.3 (decyl CH₂), 26.2 (CH₂CH₂CH₂O), 22.6 (CH₂CH₃), 14.0 (Me), 11.90 (CH₂CH₃).

Ethyl bromide, 98% (1.71 mL, 22.5 mmol, 20.0 eq) is added to a stirred solution of 5,5-bis(decyloxymethyl)-N,N,N′,N′-tetraethyl-3,7-dioxa-1,9-nonanediamine (0.69 g, 1.12 mmol) in a mixture of THF and ethanol (6 mL) (2:1). Potassium carbonate (0.13 g, 2.24 mmol, 2.0 eq) is added and the resulting mixture is refluxed for 26 h, then cooled to rt, filtered, and the filtrate is concentrated to give the title compound (5f) as a colourless crystalline solid, yield 0.78 g, 86%; R_{F} on basic alumina 0.40 (butanol, water, methanol 4:1: trace); ¹H NMR δ 0.88 ppm (t, 6H, J=6.9 Hz, 2×Me), 1.27 (br s, 28H, 14×CH₂), 1.40 (t, J=7.2 Hz, 12H, 6×Me) 1.51 (pentet, 4H, J=6.3 Hz, 2 OCH₂CH₂), 3.28 (s, 4H, decylOCH₂C), 3.33 (t, J=6.6 Hz, 4H, decyl OCH₂), 3.46 (s, 4H, CCH₂O(CH₂)₂N), 3.57 (q, 12H, J=7.2 Hz, 2 N(CH₂CH₃)₂), 3.78 (AA′ part of AA′BB′ pattern, 4H, 2 NCH₂CH₂), 3.90 (BB′ part of AA′BB′ pattern, 4H, 2OCH₂CH₂); ¹³C NMR δ 71.7 (CH₂CH₂OC), 70.9 (CCH₂O(CH₂)₂N), 69.3 (CCH₂Odecyl), 64.8 (OCH₂CH₂N), 57.5 (NCH₂CH₂), 54.2 (NCH₂CH₃), 45.0 (q C), 31.8 (CH₂CH₂CH₃), 29.5, 29.5, 29.5, 29.4, 29.2 (decyl CH₂), 26.1 (CH₂CH₂CH₂O), 22.5 (CH₂CH₃), 14.0 (Me), 8.2 (CH₂CH₃). HR ESI MS m/z calc for C₄₁H₈₈BrN₂O₄ (M+Br): 751.5927. Found: 751.5925.

### Example 5G

### 5,5-Bis(dodecyloxymethyl)-N,N,N,N′,N′,N′-hexaethyl-3,7-dioxa-1,9-nonanediammonium dibromide (5g)

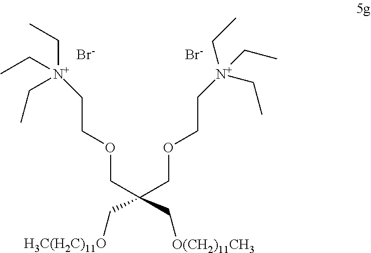

Salt 4g (Example 4G) (5.5 g, 7.4 mmol) is dissolved in a NaOH solution (2 M, 30 mL). The resulting mixture is extracted with diethyl ether (2×25 mL) to yield a colourless syrup of 5,5-bis(dodecyloxymethyl)-N,N,N′,N′-tetraethyl-3,7-dioxa-1,9-nonanediamine, yield 4.66 g, 94.1%; R_{F} 0.46 on basic alumina (dichloromethane ethanol, 98:2), ¹H NMR δ 0.88 ppm (t, 6H, J=6.9 Hz, 2×Me), 1.03 (t, J=7.14 Hz, 12H, 4×Me) 1.27 (brs, 36H, 18×CH₂), 1.52 (pentet, 4H, J=6.6 Hz, 2 OCH₂CH₂), 2.58 (q, 8H, J=7.1 Hz, 2×N(CH₂CH₃)₂), 2.67 (t, 4H, J=6.1 Hz, 2 NCH₂CH₂), 3.35 (t, J=6.6 Hz, 4H, dodecyl OCH₂), 3.35 (s, 4H, dodecylOCH₂C), 3.39 (s, 4H, CCH₂O(CH₂)₂N), 3.48 (t, J=6.1 Hz, 4H, 2 OCH₂CH₂N); ¹³C NMR δ 71.3 (CH₂CH₂OC), 70.0 (CCH₂O(CH₂)₂N), 70.0 (OCH₂CH₂N), 69.5 (CCH₂Ododecyl), 51.9 (NCH₂CH₂), 47.5 (NCH₂CH₃), 45.2 (q C), 31.9 (CH₂CH₂CH₃), 29.5, 29.4, 29.2 (dodecyl CH₂), 26.1 (CH₂CH₂CH₂O), 22.5 (CH₂CH₃), 13.9 (Me), 11.8 (CH₂CH₃).

Ethyl bromide (21.0 mL, 276 mmol, 40.0 eq) is added to a stirred solution of 5,5-bis(dodecyloxymethyl)-N,N,N′,N′-tetraethyl-3,7-dioxa-1,9-nonanediamine (4.66 g, 6.96 mmol) in a mixture of THF and ethanol (3:2) (50 mL). Potassium carbonate (2.37 g, 17.2 mmol, 2.5 eq) is added and the resulting mixture is refluxed for 33 h, cooled to rt, filtered, and the filtrate is concentrated to give the title compound (5g) as a colourless solid. Crystallization from ethyl acetate and acetone gives colourless granules: yield 4.79 g, 78.4%; mp 185° C.; R_{F} on basic alumina 0.42 (butanol, water, methanol 4:1: trace); ¹H NMR δ 0.88 ppm (t, 6H, J=6.8 Hz, 2×Me), 1.27 (brs, 36H, 18×CH₂), 1.41 (t, J=7.2 Hz, 12H, 6×Me), 1.51 (pentet, 4H, J=6.3 Hz, 2 OCH₂CH₂), 3.27 (s, 4H, dodecylOCH₂C), 3.32 (t, J=6.6 Hz, 4H, dodecyl OCH₂), 3.48 (s, 4H, CCH₂O(CH₂)₂N), 3.58 (q, 12H, J=7.2 Hz, 2 N(CH₂CH₃)₂), 3.81 (AA′ part of AA′BB′ pattern, 4H, 2 NCH₂CH₂O), 3.95 (BB′ part of AA′BB′ pattern, 4H, 2 OCH₂CH₂N); ¹³C NMR δ 71.8 (CH₂CH₂OC), 71.0 (CCH₂O(CH₂)₂N), 69.5 (CCH₂Ododecyl), 64.9 (OCH₂CH₂N), 57.6 (NCH₂CH₂O), 54.3 (NCH₂CH₃), 45.2 (q C), 31.9 (CH₂CH₂CH₃), 29.7, 29.7, 29.6, 29.4 (dodecyl CH₂), 26.3 (CH₂CH₂CH₂O), 22.7 (CH₂CH₃), 14.1 (Me), 8.3 (CH₂CH₃). HR ESI MS m/z calc for C₄₅H₉₆BrN₂O₄ (M+Br): 807.6553. Found: 807.6549

### Example 5H

### N,N,N,N′,N′,N′-Hexaethyl-3,7-dioxa-5,5-bis(tetradecyloxymethyl)-1,9-nonanediammonium dibromide (5h)

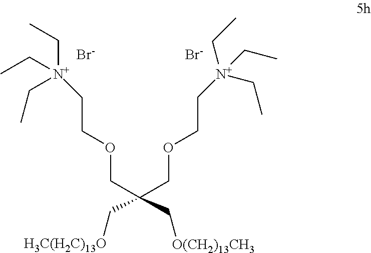

Salt 4h (Example 4H) (5.42 g, 6.79 mmol) is dissolved in a 2 M NaOH solution (30 mL). The resulting mixture is extracted with diethyl ether (2×25 mL) to yield a colourless syrup of N,N,N′,N′-tetraethyl-5,5-bis(tetradecyloxymethyl)-3,7-dioxa-1,9-nonanediamine: yield 4.11 g, 83.9%; R_{F} 0.48 on basic alumina (dichloromethane:ethanol 98:2), ¹H NMR δ 0.88 ppm (t, 6H, J=6.9 Hz, 2×Me), 1.02 (t, 12H, J=7.1 Hz, 4×Me), 1.28 (brs, 44H, 22×CH₂), 1.52 (pentet, 4H, J=6.8 Hz, 2 OCH₂CH₂), 2.57 (q, 8H, J=7.1 Hz, 2 N(CH₂CH₃)₂), 2.65 (t, 4H, J=6.2 Hz, 2 NCH₂CH₂), 3.35 (t, J=6.5 Hz, 4H, tetradecyl OCH₂), 3.35 (s, 4H, tetradecylOCH₂C), 3.39 (s, 4H, CCH₂O(CH₂)₂N), 3.48 (t, J=6.3 Hz, 4H, 2 OCH₂CH₂); ¹³C NMR δ 71.5 (CH₂CH₂OC), 70.4 (CCH₂O(CH₂)₂N), 70.3 (OCH₂CH₂N), 69.7 (CCH₂Otetradecyl), 52.1 (NCH₂CH₂), 47.8 (NCH₂CH₃), 45.4 (q C), 32.0 (CH₂CH₂CH₃), 29.8, 29.7, 29.6, 29.4 (tetradecyl CH₂), 26.3 (CH₂CH₂CH₂O), 22.8 (CH₂CH₃), 14.1 (Me), 12.1 (CH₂CH₃).

Treatment of a mixture of ethyl bromide (16.9 mL, 224 mmol, 40.0 eq) and N,N,N′,N′-tetraethyl-5,5-bis(tetradecyloxymethyl)-3,7-dioxa-1,9-nonanediamine (4.11 g, 5.66 mmol) in a THF ethanol solution (3:2) (50 mL) containing potassium carbonate (1.95 g, 14.2 mmol, 2.5 eq) as above gives the title compound (5h), as colourless solid. It is recrystallized from ethyl acetate and acetone to give colourless granules: yield 4.50 g, 85.4%; mp 180° C.; R_{F} on basic alumina 0.43 (butanol, water, methanol 4:1: trace); ¹H NMR δ 0.88 ppm (t, 6H, J=6.9 Hz, 2×Me), 1.26 (brs, 44H, 22×CH₂), 1.41 (t, J=7.2 Hz, 12H, 6×Me), 1.50 (pentet, 4H, J=6.3 Hz, 2×OCH₂CH₂), 3.27 (s, 41-1,2×tetradecylOCH₂C), 3.32 (t, J=6.6 Hz, 4H, decyl OCH₂), 3.48 (s, 4H, CCH₂O(CH₂)₂N), 3.58 (q, 12H, J=7.2 Hz, 2 N(CH₂CH₃)₂), 3.81 (AA′ part of an AA′BB′ pattern, 4H, 2 NCH₂CH₂), 3.95 (BB′ part of AA′BB′ pattern, 4H, 2 OCH₂CH₂); ¹³C NMR δ 71.7 (CH₂CH₂OC), 70.9 (CCH₂O(CH₂)₂N), 69.4 (CCH₂Otetradecyl), 64.9 (OCH₂CH₂N), 57.6 (NCH₂CH₂), 54.3 (NCH₂CH₃), 45.0 (q C), 31.8 (CH₂CH₂CH₃), 29.6, 29.6, 29.5, 29.3 (tetradecyl CH₂), 26.2 (CH₂CH₂CH₂O), 22.6 (CH₂CH₃), 14.0 (Me), 8.3 (CH₂CH₃). HR ESI MS m/z calc for C₄₉H₁₀₄BrN₂O₄ (M+Br): 863.7179. Found: 863.7178.

### Example 51

### 5,5-Bis(dodecyloxymethyl)-N,N′-diethyl-N,N,N′,N′-tetramethyl-3,7-dioxa-1,9-nonanediammonium dibromide (5i)

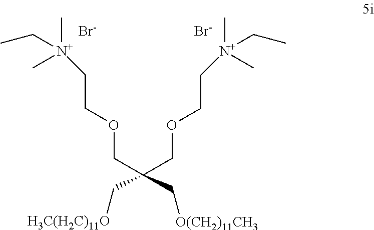

Ethyl bromide (2.3 mL, 31 mmol, 10 eq), then sodium bicarbonate (1.30 g, 15.5 mmol, 5.0 eq) are added to a stirred solution of compound 3c (Example 3C) (2.13 g, 3.10 mmol) in THF (30 mL) and the resulting mixture is refluxed for 12 h, cooled to rt, filtered, and the filtrate is concentrated to give the title compound as a colorless solid. Crystallization from ethyl acetate and acetone gives colorless granules: yield 2.37 g, 91.1%; mp 185° C.; R_{F} on basic alumina 0.45 (butanol, water, methanol 20:5:2); ¹H NMR δ 0.88 ppm (t, 6H, J=6.9 Hz, 2×Me), 1.27-1.31 (br s, 36H, 18×CH₂), 1.45 (t, J=7.2 Hz, 6H, 2×NCH₂CH₃) 1.51 (pentet, 4H, J=6.2 Hz, 2 OCH₂CH₂), 3.28 (s, 4H, dodecyl OCH₂C), 3.32 (t, J=6.6 Hz, 4H, dodecyl OCH₂), 3.43 (s, 12H, 2×N(CH₃)₂), 3.47 (s, 4H, CCH₂O(CH₂)₂N), 3.81 (q, 4H, J=7.3 Hz, 2×N(CH₂CH₃)₂), 3.92 (AA′ part of AA′ BB′ pattern, 4H, 2 OCH₂CH₂N), 3.97 (BB′ part of AA′BB′ pattern, 4H, 2 NCH₂CH₂O); ¹³C NMR δ 71.8 (CH₂CH₂OC), 70.9 (CCH₂O(CH₂)₂N), 69.4 (CCH₂Ododecyl), 65.3 (OCH₂CH₂N), 63.1 (NCH₂CH₂O), 61.0 (NCH₂CH₃) 51.3 (NCH₃)₂, 45.1 (q C), 31.9 (CH₂CH₂CH₃), 29.7, 29.7, 29.5, 29.4 (dodecyl CH₂), 26.3 (CH₂CH₂CH₂O), 22.7 (CH₂CH₃), 14.2 (Me), 8.9 (NCH₂CH₃); HR ESI MS m/z calcd for C₄₁H₈₈BrN₂O₄ (M−Br) 751.5927. found 751.5922.

### Example 5J

### 5,5-Bis(dodecyloxymethyl)-N,N,N′,N′-tetramethyl-3,7-dioxa-N,N′-dipropyl-1,9-nonanediammonium dibromide (5j)

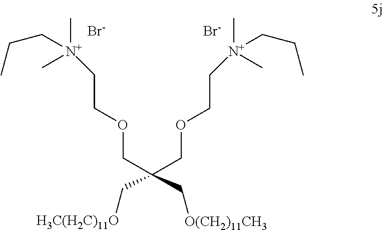

Bromopropane (5.4 mL, 59 mmol, 10 eq), then sodium bicarbonate (2.47 g, 29.4 mmol, 5.0 eq) are added to a stirred solution of compound 3c (Example 3C) (4.05 g, 5.89 mmol) in THF (50 mL) and the resulting mixture is refluxed for 26 h, cooled to rt, filtered, and the filtrate is concentrated to give the title compound as a colorless solid. Crystallization from ethyl acetate and acetone gives colorless crystals: yield 4.83 g, 95.5%; mp 62° C.; R_{F} on basic alumina 0.50 (butanol, water, methanol 20:5:2); ¹H NMR δ 0.88 ppm (t, 6H, J=6.7 Hz, 2×Me), 1.05 (t, J=7.3 Hz, 6H, 2×N(CH₂)₂CH₃) 1.26-1.31 (br s, 36H, 18×CH₂), 1.51 (pentet, 4H, J=6.2 Hz, 2 OCH₂CH₂), 1.86 (AA′ part of AA′XX′ pattern, 4H, 2×NCH₂CH₂CH₃), 3.28 (s, 4H, dodecylOCH₂C), 3.32 (t, J=6.6 Hz, 4H, dodecyl OCH₂), 3.44 (s, 12H, 2×N(CH₃)₂), 3.48 (s, 4H, CCH₂O(CH₂)₂N), 3.63 (XX′ part of AA′XX′ pattern 4H, 2×NCH₂CH₂CH₃), 3.93 (AA′ part of AA′BB′ pattern, 4H, 2, OCH₂CH₂N), 3.99 (BB′ part of AA′BB′ pattern, 4H, 2, NCH₂CH₂O); ¹³C NMR δ 71.8 (CH₂CH₂OC), 70.9 (CCH₂O(CH₂)₂N), 69.5 (CCH₂Ododecyl), 67.1 (NCH₂CH₂CH₃), 65.4 (OCH₂CH₂N), 63.6 (NCH₂CH₂O), 51.9 (NCH₃)₂, 45.2 (q C), 31.9 (CH₂CH₂CH₃), 29.7, 29.7, 29.5, 29.4 (dodecyl CH₂), 26.2 (CH₂CH₂CH₂O), 22.7 (CH₂CH₃), 16.5 (NCH₂CH₂CH₃), 14.2 (Me), 10.8 (N(CH₂)₂CH₃); HR ESI MS m/z calcd for C₄₃H₉₂BrN₂O₄ (M−Br) 779.6234. found 779.6209.

### Example 5K

### N,N′-Dibutyl-5,5-bis(dodecyloxymethyl)-N,N,N′,N′-tetramethyl-3,7-dioxa-1,9-nonanediammonium dibromide (5k)

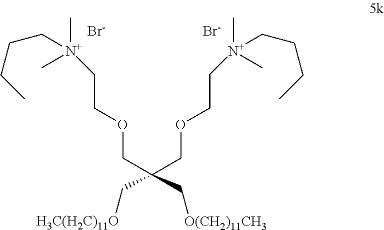

Bromobutane (4.70 mL, 44.0 mmol, 10 eq), then sodium bicarbonate (1.84 g, 22.0 mmol, 5.0 eq), are added to a stirred solution of compound 3c (Example 3C) (3.03 g, 4.4 mmol) in THF (40 mL) and the resulting mixture is refluxed for 33 h, cooled to rt, filtered, and the filtrate is concentrated to give the title compound as a colorless solid. Crystallization from ethyl acetate and methanol gives colorless crystals: yield 3.46 g, 88.7%; mp 110° C.; R_{F} on basic alumina 0.53 (butanol, water, methanol 20:5:2); ¹H NMR δ 0.88 ppm (t, 6H, J=6.9 Hz, 2×Me), 1.01 (t, J=7.3 Hz, 6H, 2×N(CH₂)₃CH₃) 1.26-1.31 (br s, 36H, 18×CH₂), 1.48 (sextet, 4H, J=7.4 Hz, 2×NCH₂CH₂CH₂CH₃), 1.51 (pentet, 4H, J=6.2 Hz, 2 OCH₂CH₂), 1.78 (AA′ part of AA′XX′ pattern, 4H, 2×NCH₂CH₂CH₂CH₃), 3.28 (s, 4H, dodecylOCH₂C), 3.32 (t, J=6.6 Hz, 4H, dodecyl OCH₂), 3.35 (s, 4H, N(CH₂)₂OCH₂C), 3.44 (s, 12H, 2×N(CH₃)₂) 3.48 (s, 4H, CCH₂O(CH₂)₂N), 3.65 (XX′ part of AA′XX′ pattern, 4H, 2×NCH₂CH₂CH₂CH₃), 3.94 (AA′ part of AA′BB′ pattern, 4H, 2 OCH₂CH₂N), 3.99 (BB′ part of AA′BB′ pattern, 4H, 2 NCH₂CH₂O); ¹³C NMR δ 71.9 (CH₂CH₂OC), 71.0 (CCH₂O(CH₂)₂N), 69.5 (CCH₂Ododecyl), 65.6 (NCH₂CH₂CH₂CH₃), 65.4 (OCH₂CH₂N), 63.7 (NCH₂CH₂O), 51.8 (NCH₃)₂, 45.2 (q C), 32.0 (CH₂CH₂CH₃), 29.8, 29.7, 29.6, 29.4 (dodecyl CH₂), 26.3 (CH₂CH₂CH₂O), 24.9 (NCH₂CH₂CH₂CH₃), 22.7 (CH₂CH₃), 14.2 (Me), 13.9 (N(CH₂)₃CH₃); HR ESI MS m/z calcd for C₄₅H₉₆BrN₂O₄ (M−Br) 807.6553. found 807.6548.

### Example 5L

### N,N,N,N′,N′,N′-Hexamethyl-6,6-bis(octyloxymethyl)-4,8-dioxa-1,11-undecanediammonium diiodide (5l)

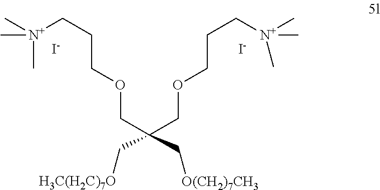

Methyl iodide (1.6 g, 11 mmol) is added to a stirred solution of amine 3e (Example 3E) (0.6 g, 1.1 mmol) in THF (50 mL) and the reaction mixture is stirred for 36 h, then allowed to cool to rt. The reaction mixture is concentrated and the residue is purified by flash chromatography using as eluant a gradient of 10 to 15% methanol in dichloromethane to give the title salt as an off-white solid: yield 0.7 g (68%); mp 212-215° C.; R_{F} 0.4 on basic alumina (8% methanol in dichloromethane); ¹H NMR (DMSO-d₆) δ 0.88 (t, 6H, J=6.5 Hz, CH₃), 1.25-1.37 (m, 20H, 10×CH₂), 1.49 (p, 4H, J=6.5 Hz, OCH₂CH₂), 1.96 (4H, AA′ part of AA′BB′ pattern, NCH₂CH₂), 3.11 (s, 18H, N(CH₃)₃), 3.31 (s, 4H, octylOCH₂C), 3.33 (t, 4H, J=6.5 Hz, OCH₂), 3.36 (s, 4H, CH₂OCH₂C), 3.38 (4H, BB′ part of AA′BB′ pattern, NCH₂), 3.42 (t, 4H, J=6.0 Hz, OCH₂); ¹³C NMR (CDCl₃) δ 70.7 (octyl CH₂O), 69.4 (OCH₂CH₂CH₂N), 68.8 (OCH₂C), 67.7 (OCH₂C), 63.3 (NCH₂), 52.3 (N(CH₃)₃), 45.0 (qC), 31.1 (OCH₂CH₂), 28.74, 28.65, 25.63 (decyl CH₂), 23.0 (NCH₂CH₂), 22.0 (CH₃CH₂), 13.9 (CH₃); HR ESI MS m/z calcd for C₃₃H₇₂IN₂O₄ (M−I) 687.4531. found 687.4522.

### Example 5M

### 6,6-Bis(decyloxymethyl)-N,N,N,N′,N′,N′-hexamethyl-4,8-dioxa-1,11-undecanediammonium diiodide (5m)

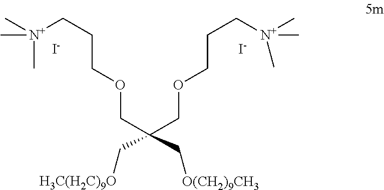

Methyl iodide (1.3 g, 9.5 mmol) is added to a stirred solution of amine 3f (Example 3F) (0.7 g, 1.2 mmol) in THF (70 mL) and the reaction mixture is stirred for 36 h, then allowed to cool to rt. The reaction mixture is concentrated and the residue is purified by flash chromatography using as eluant a gradient of 10 to 15% methanol in dichloromethane to give compound 5m as an off-white solid: yield 0.7 g (68%); mp 219-222° C.; R_{F} 0.5 on basic alumina (7% methanol in dichloromethane); ¹H NMR (CDCl₃) δ 0.88 (t, 6H, J=6.5 Hz, CH₃), 1.22-1.35 (m, 28H, 14×CH₂), 1.50 (p, 4H, J=6.5 Hz, OCH₂CH₂), 2.10 (m, 4H, NCH₂CH₂), 3.31 (s, 4H, decylOCH₂C), 3.36 (t, 4H, J=6.5 Hz, OCH₂), 3.41 (s, 4H, CH₂OCH₂C), 3.48 (s, 18H, N(CH₃)₃), 3.60 (t, 4H, J=6.0 Hz, OCH₂), 3.85 (m, 4H, NCH₂); ¹³C NMR (CDCl₃) δ 71.8 (decyl CH₂O), 70.3 (OCH₂CH₂CH₂N), 69.3 (OCH₂C), 67.8 (OCH₂C), 65.2 (NCH₂), 54.2 (N(CH₃)₃), 45.6 (q, C), 32.0 (OCH₂CH₂), 29.87, 29.81, 29.72, 28.51, 26.4 (decyl CH₂), 24.3 (NCH₂CH₂), 22.8 (CH₂CH₃), 14.2 (CH₃); HR ESI MS m/z calcd for C₃₇H₈₀IN₂O₄(M−I) 743.5157. found 743.5130.

### Example 5N

### 6,6-Bis(dodecyloxymethyl)-N,N,N,N′,N′,N′-hexamethyl-4,8-dioxa-1,11-undecanediammonium diiodide (5n)

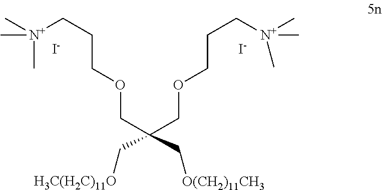

Methyl iodide (1.5 g, 11 mmol) is added to a stirred solution of amine 3g (Example 3G) (0.70 g, 1.1 mmol) in THF (70 mL) and the reaction mixture is stirred for 36 h, then allowed to cool to rt. The reaction mixture is concentrated and the residue is purified by flash chromatography using as eluant a gradient of 10 to 15% methanol in dichloromethane to give compound 5n as an off-white solid: yield 0.82 g (82%); mp 230-234° C.; R_{F} 0.58 on basic alumina (7% methanol in dichloromethane); ¹H NMR (CDCl₃) δ 0.88 (t, 6H, J=6.5 Hz, CH₃), 1.24-1.35 (m, 36H, 18×CH₂), 1.51 (p, 4H, J=6.5 Hz, OCH₂CH₂), 2.10 (m, 4H, NCH₂CH₂), 3.31 (s, 4H, decylOCH₂C), 3.35 (t, 4H, J=6.5 Hz, OCH₂), 3.40 (s, 4H, CH₂OCH₂C), 3.48 (s, 18H, N(CH₃)₃), 3.60 (t, 4H, J=6.0 Hz, OCH₂), 3.82 (m, 4H, NCH₂); ¹³C NMR (CDCl₃) δ 71.8 (dodecyl CH₂O), 70.4 (OCH₂CH₂CH₂N), 69.4 (OCH₂C), 67.9 (OCH₂C), 65.3 (NCH₂), 54.3 (N(CH₃)₃), 45.7 (qC), 32.1 (OCH₂CH₂), 29.89, 29.74, 29.54, 26.4 (decyl CH₂), 24.4 (NCH₂CH₂), 22.9 (CH₃CH₂), 14.3 (CH₃); HR ESI MS m/z calcd for C₄₁H₈₈IN₂O₄ (M−I) 799.5783. found 799.5815.

### Example 5O

N,N,N,N′,N′,N′-Hexamethyl-4,8-dioxa-6,6-bis(tetradecyloxymethyl)-1,11-undecanediammonium diiodide (5o)

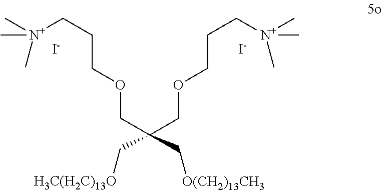

Methyl iodide (3.1 g, 22 mmol) is added to a stirred solution of amine 3h (Example 3H) (1.50 g, 2.23 mmol) in THF (100 mL) and the reaction mixture is stirred for 36 h then allowed to cool to rt. The reaction mixture is concentrated and the solid residue is crystallized from dichloromethane to give pure compound 5o as a shiny white solid: yield 1.5 g (70%); mp 222-225° C.; R_{F} 0.6 on basic alumina (7% methanol in dichloromethane); ¹H NMR (CDCl₃) δ 0.88 (t, 6H, J=6.5 Hz, CH₃), 1.22-1.37 (m, 44H, 22×CH₂), 1.51 (p, 4H, J=6.5 Hz, OCH₂CH₂), 2.09 (m, 4H, NCH₂CH₂), 3.31 (s, 4H, decylOCH₂C), 3.35 (t, 4H, J=6.5 Hz, OCH₂), 3.41 (s, 4H, CH₂OCH₂C), 3.48 (s, 18H, N(CH₃)₃), 3.61 (t, 4H, J=6.0 Hz, OCH₂), 3.90 (m, 4H, NCH₂); ¹³C NMR (CDCl₃) δ 71.8 (tetradecyl CH₂O), 70.4 (OCH₂CH₂CH₂N), 69.4 (OCH₂C), 67.8 (OCH₂C), 65.2 (NCH₂), 54.2 (N(CH₃)₃), 45.7 (qC), 32.1 (OCH₂CH₂), 29.87, 29.72, 29.51, 26.4 (tetradecyl CH₂), 24.4 (NCH₂CH₂), 22.8 (CH₃CH₂), 14.3 (CH₃); HR ESI MS m/z calcd for C₄₅H₉₆IN₂O₄ (M−I) 855.6409. found 855.6432.

### Example 6A

### Diethyl 3,13-diazonia-3,3,13,13-tetramethyl-8,8-bis(octyloxymethyl)-6,10-dioxa-pentadecanedioate dibromide (6a)

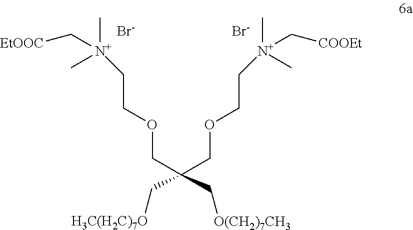

A solution of compound 3a (Example 3A) (0.61 g, 1.21 mmol) and ethyl bromoacetate (0.31 mL, 2.79 mmol, 2.3 eq) in diethyl ether (20 mL) is stirred for 26 h, to give a colorless solid that is isolated by filtration, then washed with ether. The solid is crystallized from ethyl acetate and methanol to give the title compound as a colorless crystalline solid: yield 0.88 g, 84%; mp 110-111° C.; R_{F} on basic alumina 0.59 (butanol:water:methanol 20:5:2); ¹H NMR δ 0.88 (t, 6H, J=6.9 Hz, 2×Me), 1.27 (brs, 20H, 10×CH₂), 1.32 (t, 6H, J=7.2 Hz, 2×Me), 1.50 (pentet, 4H, J=6.0 Hz, 2 OCH₂CH₂), 3.28 (s, 4H, octyl OCH₂C), 3.33 (t, 4H, J=6.6 Hz, octyl OCH₂C), 3.50 (s, 4H, CCH₂O(CH₂)₂N), 3.71 (s, 12H, 2N(CH₃)₂), 3.96 (br AA′ part of AA′BB′ pattern, 4H, 2 OCH₂CH₂N), 4.27 (q, 4H, J=7.2 Hz, OCH₂CH₃), 4.32 (br BB′ part of AA′BB′ pattern, 4H, 2 NCH₂CH₂O), 4.96 (s, 4H, CH₂COOR); ¹³C NMR δ 164.9 (CH₂COOR), 71.8 (CH₂CH₂OC), 71.1 (CCH₂O(CH₂)₂N), 69.4 (CCH₂O octyl), 65.4 (OCH₂CH₂N), 64.1 (NCH₂CH₂O), 62.7 (CH₂COOCH₂CH₃), 62.3 (CH₂COOCH₂CH₃), 52.4 (N(CH₃)₂), 45.1 (qC), 32.0 (CH₂CH₂CH₃), 29.6, 29.5, 29.3, (octyl CH₂), 26.2 (CH₂CH₂CH₂O), 22.7 (CH₂CH₃), 14.12 (Me), 14.06 (CH₃CH₂); HR ESI MS m/z calc for C₃₇H₇₆N₂O₈/2 ((M−2Br)/2) 338.2795. found 338.2811.

### Example 6B

### Diethyl 3,13-diazonia-8,8-bis(decyloxymethyl)-3,3,13,13-tetramethyl-6,10-dioxapentadecanedioate dibromide (6b)

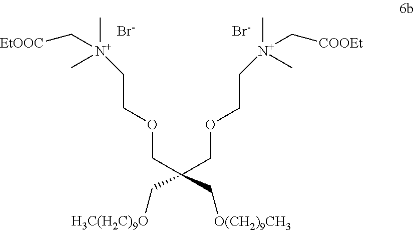

A solution of compound 3b (Example 3B) (1.86 g, 3.33 mmol) and ethyl bromoacetate (0.85 mL, 7.66 mmol, 2.3 eq) in diethyl ether (35 mL) is stirred for 26 h then filtered and concentrated to give a colorless solid that is crystallized from ethyl acetate and methanol to give the title compound as a colorless crystalline solid, yield: 2.66 g, 90%; mp 114-115° C.; R_{F} on basic alumina 0.61 (butanol:water:methanol 20:5:2); ¹H NMR δ 0.88 (t, 6H, J=6.9 Hz, 2×Me), 1.27 (brs, 28H, 14×CH₂), 1.32 (t, 6H, J=7.2 Hz, 2×Me), 1.50 (pentet, 4H, J=6.0 Hz, 2 OCH₂CH₂), 3.27 (s, 4H, decyl, OCH₂C), 3.32 (t, 4H, J=6.5 Hz, decyl OCH₂C), 3.49 (s, 4H, CCH₂O(CH₂)₂N), 3.71 (s, 12H, 2N(CH₃)₂), 3.94 (br AA′ part of AA′BB′ pattern, 4H, 2 OCH₂CH₂N), 4.27 (q, 4H, J=7.2 Hz, OCH₂CH₃), 4.31 (br BB′ part of AA′BB′ pattern, 4H, 2 NCH₂CH₂O), 4.96 (s, 4H, CH₂COOR); ¹³C NMR δ 165.0 (CH₂COOR), 71.9 (CH₂CH₂OC), 71.1 (CCH₂O(CH₂)₂N), 69.4 (CCH₂O decyl), 65.4 (OCH₂CH₂N), 64.3 (NCH₂CH₂O), 62.8 (CH₂COOCH₂CH₃), 62.4 (CH₂COOCH₂CH₃), 52.5 (N(CH₃)₂), 45.2 (qC), 32.0 (CH₂CH₂CH₃), 29.8, 29.7, 29.6, 29.5 (decylCH₂), 26.4 (CH₂CH₂CH₂O), 22.8 (CH₂CH₃), 14.2 (Me), 14.1 (CH₃CH₂); HR ESI MS m/z calc for C₄₁H₈₄N₂O₈/2 ((M−2Br)/2) 366.3108. found 366.3112.

### Example 6C

### Diethyl 3,13-diazonia-8,8-bis(dodecyloxymethyl)-3,3,13,13-tetramethyl-6,10-dioxapentadecanedioate dibromide (6c)

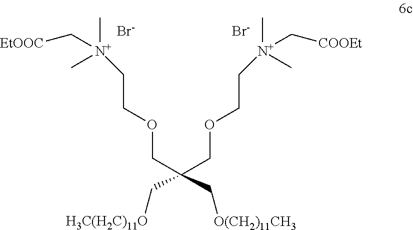

A solution of compound 3c (Example 3C) (1.77 g, 2.88 mmol) and ethyl bromoacetate (0.73 mL, 6.60 mmol, 2.3 eq) in diethyl ether (35 mL) is treated following the procedure of Example 6A to give the title compound as a colorless solid that is crystallized from ethyl acetate and methanol to give a colorless crystalline solid, yield: 2.05 g, 73%; mp 119-120° C.; R_{F} on basic alumina 0.63 (butanol:water:methanol 20:5:2); ¹H NMR δ 0.88 (t, 6H, J=6.9 Hz, 2×Me), 1.26 (brs, 36H, 18×CH₂), 1.32 (t, 6H, J=7.2 Hz, 2×Me), 1.51 (pentet, 4H, J=6.4 Hz, 2 OCH₂CH₂), 3.27 (s, 4H, dodecyl, OCH₂C), 3.33 (t, 4H, J=6.6 Hz, dodecyl, OCH₂C), 3.49 (s, 4H, CCH₂O(CH₂)₂N), 3.70 (s, 12H, 2N(CH₃)₂), 3.95 (br AA′ part of AA′BB′ pattern, 4H, 2 OCH₂CH₂N), 4.26 (q, 4H, J=7.2 Hz, OCH₂CH₃), 4.31 (br BB′ part of AA′BB′ pattern, 4H, 2 NCH₂CH₂O), 4.94 (s, 4H, CH₂COOR); ¹³C NMR δ 164.9 (CH₂COOR), 71.8 (CH₂CH₂OC), 71.1 (CCH₂O(CH₂)₂N), 69.4 (CCH₂O dodecyl), 65.4 (OCH₂CH₂N), 64.1 (NCH₂CH₂O), 62.7 (CH₂COOCH₂CH₃), 62.3 (CH₂COOCH₂CH₃), 52.4 (N(CH₃)₂), 45.1 (qC), 32.0 (CH₂CH₂CH₃), 29.7, 29.5, 29.4 (dodecylCH₂), 26.3 (CH₂CH₂CH₂O), 22.7 (CH₂CH₃), 14.12 (Me), 14.06 (CH₃CH₂); HR ESI MS m/z calcd for C₄₅H₉₂N₂O₈/2 ((M−2Br)/2) 394.3421. found 394.3424.

### Example 6D

### Diethyl 3,13-diazonia-3,3,13,13-tetramethyl-6,10-dioxa-8,8-bis(tetradecyloxymethyl)pentadecanedioate dibromide (6d)

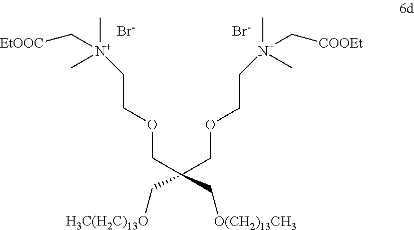

A solution of compound 3d (Example 3D) (0.78 g, 1.22 mmol) and ethyl bromoacetate (0.31 mL, 2.80 mmol, 2.3 eq) in diethyl ether (25 mL) is treated following the procedure of Example 6A to give the title compound (6d) as a colorless solid that precipitated from ethyl acetate and methanol to give a colorless amorphous solid, yield: 0.89 g, 71%; mp 116-117° C.; R_{F} on basic alumina 0.66 (butanol:water:methanol 20:5:2); ¹H NMR δ 0.88 (t, 6H, J=6.9 Hz, 2×Me), 1.26 (brs, 44H, 22×CH₂), 1.32 (t, 6H, J=7.2 Hz, 2×Me), 1.50 (pentet, 4H, J=6.0 Hz, 2 OCH₂CH₂), 3.27 (s, 4H, tetradecyl, OCH₂C), 3.32 (t, 4H, J=6.6 Hz, tetradecyl, OCH₂C), 3.48 (s, 4H, CCH₂O(CH₂)₂N), 3.71 (s, 12H, 2N(CH₃)₂), 3.93 (br AA′ part of AA′BB′ pattern, 4H, 2 OCH₂CH₂N), 4.26 (q, 4H, J=7.2 Hz, COCH₂CH₃), 4.30 (br BB′ part of AA′BB′ pattern, 4H, 2 NCH₂CH₂O), 4.95 (s, 4H, CH₂COOR); ¹³C NMR δ 164.9 (CH₂COOR), 71.8 (CH₂CH₂OC), 71.1 (CCH₂O(CH₂)₂N), 69.4 (CCH₂O tetradecyl), 65.4 (OCH₂CH₂N), 64.2 (NCH₂CH₂O), 62.7 (CH₂COOCH₂CH₃), 62.4 (CH₂COOCH₂CH₃), 52.5 (N(CH₃)₂), 45.1 (qC), 32.0 (CH₂CH₂CH₃), 29.8, 29.7, 29.6, 29.4 (tetradecyl CH₂), 26.3 (CH₂CH₂CH₂O), 22.8 (CH₂CH₃), 14.2 (Me), 14.1 (CH₃CH₂O); HR ESI MS m/z calcd for C₄₉H₁₀₀N₂O₈/2 ((M−2Br)/2) 422.3734. found 422.3721.

### Example 7A

### 3,13-Diazonia-3,3,13,13-tetramethyl-8,8-bis(octyloxymethyl)-6,10-dioxapentadecanedioate (7a)

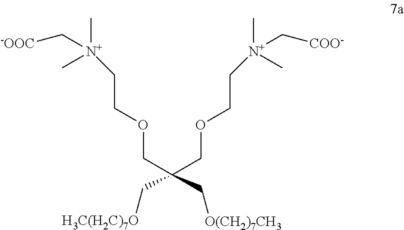

Compound 6a (Example 6A) (0.44 g, 0.51 mmol) and IRA-400 anion-exchange resin (OH⁻) (11.0 g) in ethanol (30 mL) are stirred at rt for 24 h. The reaction mixture is filtered and the filtrate is concentrated to a semi-solid residue that precipitated from ethyl acetate and methanol to give the title compound as a colorless waxy mass, yield: 0.28 g, 90%; mp 183° C.; R_{F} on basic alumina 0.37 (butanol:water:methanol 20:5:2); ¹H NMR δ 0.88 (t, 6H, J=6.7 Hz, 2×Me), 1.28-1.32 (brs, 20H, 10×CH₂), 1.51 (pentet, 4H, J=6.2 Hz, 2 OCH₂CH₂), 3.29 (s, 4H, octyl OCH₂C), 3.33 (t, 4H, J=6.4 Hz, octyl OCH₂), 3.39 (s, 4H, CCH₂O(CH₂)₂N), 3.39 (s, 12H, 2N(CH₃)₂), 3.82 (br AA′ part of AA′BB′ pattern, 4H, 2 NCH₂CH₂O), 3.99 (BB′ part of AA′BB′ pattern, 4H, 2 OCH₂CH₂N), 3.99 (s, 4H, CH₂COO⁻); ¹³C NMR δ 166.2 (CH₂COO⁻), 71.4 (CH₂CH₂OC), 70.6 (CCH₂O(CH₂)₂N), 69.2 (CCH₂O octyl), 65.46 (NCH₂CH₂O), 65.3 (OCH₂CH₂N), 62.4 (CH₂COO⁻), 52.0 (N (CH₃)₂), 44.7 (q C), 31.6 (CH₂CH₂CH₃), 29.3, 29.2, 29.1 (octyl CH₂), 25.9 (CH₂CH₂CH₂O), 22.4 (CH₂CH₃), 13.8 (Me); HR ESI MS m/z calcd for C₃₃H₆₇N₂O₈ (M+H) 619.4892. found 619.4859.

### Example 7B

### 3,13-Diazonia-8,8-bis(decyloxymethyl)-3,3,13,13-tetramethyl-6,10-dioxapentadecanedioate (7b)

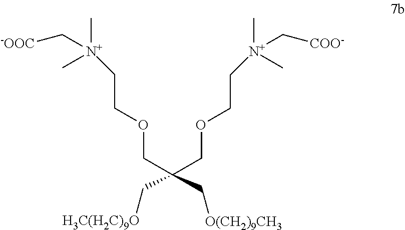

Compound 6b (Example 6B) (2.21 g, 2.55 mmol) and IRA-400 anion-exchange resin (OH⁻) (11.6 g) in ethanol (40 mL) are stirred at rt following the procedure of Example 7A. The reaction mixture is filtered and concentrated to a semi-solid residue that precipitated from ethyl acetate and methanol to give the title compound as a colorless waxy solid, yield: 1.47 g, 89%; mp 175° C.; R_{F} on basic alumina 0.35 (butanol:water:methanol 20:5:2); ¹H NMR δ 0.88 (t, 6H, J=6.9 Hz, 2×Me), 1.21-1.32 (brs, 28H, 14×CH₂), 1.51 (pentet, 4H, J=6.5 Hz, 2 OCH₂CH₂), 3.28 (s, 4H, decylOCH₂C), 3.33 (t, 4H, J=6.4 Hz, decyl OCH₂), 3.38 (s, 12H, 2N(CH₃)₂), 3.39 (s, 4H, CCH₂O(CH₂)₂N), 3.81 (br AA′ part of AA′BB′ pattern, 4H, 2 NCH₂CH₂O), 3.95 (BB′ part of AA′BB′ pattern, 4H, 2 OCH₂CH₂N), 4.12 (s, 4H, CH₂COO⁻); ¹³C NMR δ 167.9 (CH₂COO⁻), 71.7 (CH₂CH₂OC), 70.8 (CCH₂O(CH₂)₂N), 69.3 (CCH₂Odecyl), 65.6 (NCH₂CH₂O), 65.5 (OCH₂CH₂N), 63.1 (CH₂COO⁻), 52.1 (N(CH₃)₂), 45.1 (q C), 32.0 (CH₂CH₂CH₃), 29.7, 29.7, 29.6, 29.4 (decyl CH₂), 26.3 (CH₂CH₂CH₂O), 22.7 (CH₂CH₃), 14.1 (Me); HR ESI MS m/z calcd for C₃₇H₇₄N₂O₈Na (M+Na) 697.5337. found 697.5304.

### Example 7C

### 3,13-Diazonia-8,8-bis(dodecyloxymethyl)-3,3,13,13-tetramethyl-6,10-dioxapentadecanedioate (7c)

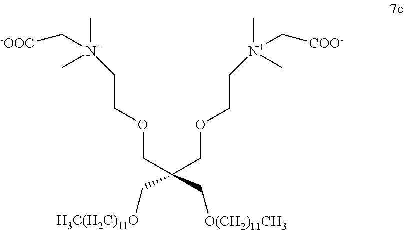

Compound 6c (Example 6C) (1.54 g, 1.62 mmol) and IRA-400 anion-exchange resin (OH⁻) (12.0 g) in ethanol (50 mL) are stirred at rt following the procedure of Example 7A. The reaction mixture is filtered and concentrated to a semi-solid residue that is crystallized from ethyl acetate and methanol to give the title compound (7c) as colorless granules: yield 1.1 g, 93%; mp 170-171° C.; R_{F} on basic alumina 0.33 (butanol:water:methanol 20:5:2); ¹H NMR δ 0.88 (t, 6H, J=6.9 Hz, 2×Me), 1.26 (brs, 36H, 18×CH₂), 1.51 (pentet, 4H, J=6.5 Hz, 2 OCH₂CH₂), 3.28 (s, 4H, dodecyl OCH₂C), 3.33 (t, 4H, J=6.4 Hz, dodecyl OCH₂), 3.39 (s, 12H, 2N(CH₃)₂) 3.4 (s, 4H, CCH₂O(CH₂)₂N), 3.83 (br AA′ part of AA′BB′ pattern, 4H, 2 NCH₂CH₂O), 3.99 (BB′ part of AA′BB′ pattern, 4H, 2 OCH₂CH₂N), 3.99 (s, 4H, CH₂COO⁻; ¹³C NMR δ 166.3 (CH₂COO⁻), 71.8 (CH₂CH₂OC), 70.9 (CCH₂O(CH₂)₂N), 69.5 (CCH₂O dodecyl), 65.7 (NCH₂CH₂O), 65.5 (OCH₂CH₂N), 62.7 (CH₂COO⁻), 52.4 (N(CH₃)₂), 45.1 (q C), 32.0 (CH₂CH₂CH₃), 29.7, 29.6, 29.4 (dodecyl CH₂), 26.3 (CH₂CH₂CH₂O), 22.7 (CH₂CH₃), 14.1 (Me); HR ESI MS m/z calcd for C₄₁H₈₃N₂O₈ (M+H) 731.6144. found 731.6117.

### Example 7D

### 3,13-Diazonia-3,3,13,13-tetramethyl-6,10-dioxa-8,8-bis(tetradecyloxymethyl)pentadecanedioate (7d)

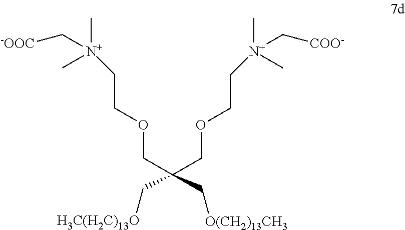

Compound 6d (Example 6D) (0.89 g, 0.88 mmol) and IRA-400 anion-exchange resin (OH⁻) (11.2 g) in ethanol (30 mL) are stirred at rt for 24 h. The reaction mixture is filtered and the filtrate is concentrated to a colorless semi-solid residue that is crystallized from ethyl acetate and methanol to give the title compound as a colorless powder: yield 0.65 g, 92%; mp 168° C.; R_{F} on basic alumina 0.30 (butanol:water:methanol 20:5:2); ¹H NMR δ 0.88 (t, 6H, J=6.9 Hz, 2×Me), 1.26-1.31 (brs, 44H, 22×CH₂), 1.50 (pentet, 4H, J=6.2 Hz, 2 OCH₂CH₂), 3.28 (s, 4H, tetradecylOCH₂C), 3.33 (t, 4H, J=6.4 Hz, tetradecyl OCH₂), 3.40 (s, 12H, 2N(CH₃)₂), 3.40 (s, 4H, CCH₂O(CH₂)₂N), 3.83 (br AA′ part of AA′BB′ pattern, 4H, 2 NCH₂CH₂O), 3.98 (BB′ part of AA′ BB′ pattern, 4H, 2 OCH₂CH₂N), 4.10 (s, 4H, CH₂COO⁻); ¹³C NMR δ 166.1 (CH₂COO⁻), 71.8 (CH₂CH₂OC), 70.9 (CCH₂O(CH₂)₂N), 69.5 (CCH₂Odecyl), 65.7 (NCH₂CH₂O), 65.5 (OCH₂CH₂N), 62.9 (CH₂COO⁻), 52.4 (N(CH₃)₂), 45.2 (q C), 32.1 (CH₂CH₂CH₃), 29.9, 29.8, 29.7, 29.5 (tetradecyl CH₂), 26.4 (CH₂CH₂CH₂O), 22.8 (CH₂CH₃), 14.2 (Me); HR ESI MS m/z calcd for C₄₅H₉₀N₂O₈Na (M+Na) 809.6589. found 809.6567.

### Example 8A

### 1,3-Diiodo-2,2-bis(octyloxymethyl)propane (8a)

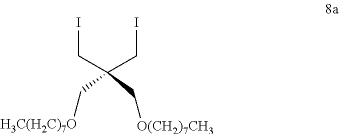

Iodine (10.2 g, 0.0415 mol, 2.5 eq), imidazole (2.73 g, 0.0415 mol, 2.5 eq) and triphenylphosphine (8.94 g, 0.0353 mol, 2.2 eq) are added to a solution of compound 2a (Example 2A) (5.80 g, 0.0161 mol) in anhydrous toluene (200 mL) and the reaction mixture is refluxed for 3 h. More iodine is then added to consume excess triphenylphosphine and reflux is continued for 1 h. The cooled reaction mixture is stirred for 10 min each with saturated sodium bicarbonate (100 mL) and 10% aqueous sodium thiosulfate (200 mL) solutions. The organic layer is washed with water (3×50 mL), dried (MgSO₄) and concentrated. The residue is taken up in hexanes and the solution is passed a short silica gel column. Concentration gives the title compound (8a) as a colorless oil: 7.89 g, 86%; R_{F} 0.34 (98:2 hexanes:dichloromethane); ¹H NMR δ 0.89 (t, 6H, J=6.3 Hz, 2×Me), 1.22-1.36 (br s, 20H, 10×CH₂), 1.55 (pentet, 4H, J=6.1 Hz, 2 OCH₂CH₂), 3.33 (s, 4H, CH₂I), 3.35 (s, 4H, OCH₂C), 3.42 (t, 4H, dodecyl OCH₂); ¹³C NMR δ 71.7 (CH₂CH₂OC), 70.9 (CCH₂OCH₂C), 41.7 (q C), 32.0 (CH₂CH₂CH₃), 29.69, 29.56, 29.47 (3 octyl CH₂), 26.3 (CH₂CH₂CH₂O), 22.8 (CH₂CH₃), 14.3 (Me), 12.1 (CH₂I); HR ESI MS; m/z calc for C₂₁H₄₃I₂O₂ (M+H) 581.1353. found 581.1351.

### Example 8B

### 1,3-Diiodo-2,2-bis(decyloxymethyl)propane (8b)

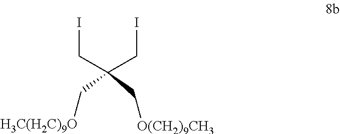

A solution of compound 2b (Example 2B) (10.0 g, 24.3 mmol) in toluene (300 mL) is refluxed with iodine (16.3 g, 25.9 mmol, 2.7 eq), imidazole (4.08 g, 60.0 mmol, 2.5 eq) and triphenylphosphine (13.4 g, 50.4 mmol, 2.1 eq) following the procedure of Example 8A to give the title compound (8b) as an oil: 14.4 g, 94%; R_{F} 0.43 (98:2 hexanes:dichloromethane); ¹H NMR δ 0.88 (t, 6H, J=6.9 Hz, 2×Me), 1.22-1.36 (br s, 28H, 14×CH₂), 1.54 (pentet, 4H, J=6.8 Hz, 2 CH₂CH₂O), 3.32 (s, 4H, CH₂I), 3.35 (s, 4H, OCH₂C), 3.41 (t, 4H, J=6.5 Hz, 2 decyl OCH₂); ¹³C NMR δ 71.7 (CH₂CH₂OC), 70.9 (CCH₂OCH₂C), 41.7 (q C), 32.1 (CH₂CH₂CH₃), 29.65, 29.63, 29.48, 29.37 (6 dodecyl CH₂), 29.58 (OCH₂CH₂), 26.3 (CH₂CH₂CH₂O), 22.8 (CH₂CH₃), 14.3 (Me), 12.1 (CH₂I); HR ESI MS; m/z calc for C₂₅H₅₁I₂O₂ 637.1979. found 637.1976.

### Example 8C

### 1,3-Diiodo-2,2-bis(dodecyloxymethyl)propane (8c)

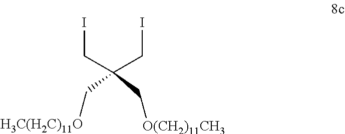

Treatment of compound 2c (Example 2C) (10.0 g, 21.2 mmol) in toluene (300 mL) with iodine (14.0 g, 52.8 mmol, 2.5 eq), imidazole (3.58 g, 52.8 mol, 2.5 eq) and triphenylphosphine (13.82 g, 52.8 mol, 2.5 eq) following the procedure of Example 8A gives the title compound (8c) as an oil: 14.0 g, 95%; R_{F} 0.44 (98:2 hexanes: dichloromethane); ¹H NMR δ 0.88 (t, 6H, J=6.5 Hz, 2×Me), 1.22-1.36 (br s, 36H, 18×CH₂), 1.55 (pentet, 4H, J=6.4 Hz, 2 OCH₂CH₂), 3.32 (s, 4H, CH₂I), 3.35 (s, 4H, OCH₂C), 3.41 (t, 4H, dodecyl OCH₂); ¹³C NMR δ 71.7 (CH₂CH₂OC), 70.9 (CCH₂OCH₂C), 41.7 (q C), 32.1 (CH₂CH₂CH₃), 29.92, 3×29.88, 29.79, 29.59 (6 dodecyl CH₂), 29.68 (OCH₂CH₂), 26.3 (CH₂CH₂CH₂O), 22.8 (CH₂CH₃), 14.3 (Me), 12.1 (CH₂I); HR ESI MS; m/z calc for C₂₉H₅₉O₄ 693.2605. found 693.2605.

### Example 8D

### 1,3-Diiodo-2,2-bis(tetradecyloxymethyl)propane (8d)

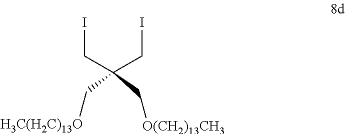

Treatment of compound 2d (Example 2D) (5.0 g, 9.5 mmol) in toluene (200 mL) with iodine (7.20 g, 28.4 mmol, 3.0 eq), imidazole (1.60 g, 23.6 mmol, 2.5 eq) and triphenylphosphine (7.19 g, 28.4 mmol, 2.9 eq) following the procedure of Example 8A gives the title compound (8d) as a colourless solid: yield 6.36 g, 90%; mp 27-28° C.; R_{F} 0.45 (98:2 hexanes:dichloromethane); ¹H NMR δ 0.88 (t, 6H, J=7.0 Hz, 2×Me), 1.22-1.36 (br s, 44H, 22×CH₂), 1.53 (pentet, 4H, J=6.9 Hz, 2 OCH₂CH₂), 3.33 (s, 4H, CH₂I), 3.35 (s, 4H, OCH₂C), 3.41 (t, 4H, tetradecyl OCH₂); ¹³C NMR δ 71.8 (CH₂CH₂OC), 71.0 (CCH₂OCH₂C), 41.8 (q C), 32.1 (CH₂CH₂CH₃), 29.87, 29.86, 29.85, 29.82, 2×29.81, 29.72, 29.52 (8 tetradecyl CH₂), 29.61 (OCH₂CH₂), 26.4 (CH₂CH₂CH₂O), 22.9 (CH₂CH₃), 14.3 (Me), 12.1 (CH₂I); LR ESI m/z calc for C₃₃H₆₇I₂O₂ 749.32. found 749.1. Anal. Calc. for C₃₃H₆₆I₂O₂: C, 52.94; H, 8.89. Found: C, 53.14; H, 9.20.

### Example 9A

### N,N,N′,N′-Tetramethyl-2,2-bis(octyloxymethyl)-1,3-propanediaminium dichloride (9a)

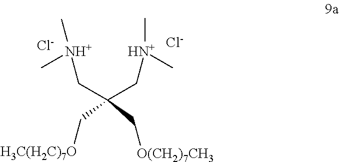

Compound 8a (Example 8A) (9.98 g, 17.2 mmol), dimethylamine in THF (2 M, 87 mL, 0.17 mol, 10 eq) and potassium carbonate (5.9 g, 43 mmol, 2.5 eq) are added to a sealed tube with the aid of THF (10 mL). The reaction mixture is stirred at 160-170° C. After one week, all of the starting material has been consumed and the reaction mixture is filtered and the solvent is removed in vacuo at 35-40° C. to give a yellow oil, 1,3-bis(dimethylamino)-2,2-bis(octyloxymethyl)propane. The crude oil is taken up in dichloromethane (50 mL) and the resulting solution is shaken with ice cold 2 M HCl (75 mL). The aqueous layer is extracted with dichloromethane (2×50 mL), then the combined organic layers are washed with water (20 mL), dried (MgSO₄), and concentrated to give the title compound (9a) as a light yellow crystalline solid that is recrystallized from ethyl acetate, acetone 15:1 to give clear rectangular crystals: yield 5.62 g, 67.3%; mp 145° C.; R_{F} 0.29 on basic alumina (hexanes, ethyl acetate, methanol 96:4:0.4); ¹H NMR δ 0.88 ppm (t, 6H, J=6.9 Hz, 2×Me), 1.26-1.33 (br s, 20H, 10×CH₂), 1.56 (pentet, 4H, J=6.6 Hz, 2 OCH₂CH₂), 2.97 (s, 12H, 2×N(CH₃)₂), 3.47 (t, 4H, J=6.6 Hz, octyl OCH₂); 3.68 (s, 4H, OCH₂C), 3.79 (s, 4H, CH₂N), 11.78 (brs, HN); ¹³C NMR δ 71.7 (CH₂CH₂OC), 66.9 (CCH₂OCH₂C), 58.6 (CH₂N), 47.5 (N(CH₃)₂), 44.5 (q C), 31.8 (CH₂CH₂CH₃), 29.5, 29.3, 29.2, (3 octyl CH₂), 26.2 (CH₂CH₂CH₂O), 22.6 (CH₂CH₃), 14.1 (Me); HR ESI MS: m/z calc for C₂₅H₅₅N₂O₂ (M−H−2Cl): 415.4264. Found 415.4260.

### Example 9B

### 2,2-Bis(decyloxymethyl)-N,N,N′,N′-tetramethyl-1,3-propanediaminium dichloride (9b)

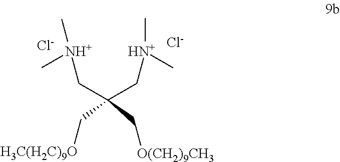

Compound 8b (Example 8B) (6.88 g, 10.8 mmol), dimethylamine in THF (2M, 54 mL, 0.11 mol, 10 eq), and potassium carbonate (3.72 g, 27.0 mmol, 2.5 eq) are added to a sealed tube with the aid of THF (10 mL). The mixture is heated in sealed tube following the procedure of Example 9A to give a yellow oil, 1,3-bis(dimethylamino)-2,2-bis(decyloxymethyl)propane that is taken up in dichloromethane (30 mL). This solution is shaken with ice cold 2 M HCl (30 mL) following the procedure of Example 9A to give colourless crystals that are recrystallized from ethyl acetate/acetone 2/1 to give colorless needles: yield 3.95 g, 67.5%; mp 130-133° C.; R_{F} 0.32 on basic alumina (hexanes, ethyl acetate, methanol 96:4:0.4); ¹H NMR δ 0.88 ppm (t, 6H, J=6.9 Hz, 2×Me), 1.26-1.32 (br s, 28H, 14×CH₂), 1.55 (pentet, 4H, J=6.6 Hz, 2 OCH₂CH₂), 2.97 (s, 12H, 2×N(CH₃)₂), 3.47 (t, 4H, J=6.6 Hz, decyl OCH₂), 3.67 (s, 4H, OCH₂C), 3.81 (s, 4H, CH₂N), 11.85 (brs, HN); ¹³C NMR δ 71.8 (CH₂CH₂OC), 66.9 (CCH₂OCH₂C), 58.6 (CH₂N), 47.7 (N(CH₃)₂), 44.6 (q C), 32.0 (CH₂CH₂CH₃), 29.7, 29.7, 29.6, 29.5, 29.4, (decyl CH₂), 26.3 (CH₂CH₂CH₂O), 22.8 (CH₂CH₃), 14.2 (Me); HR ESI MS m/z calc for C₂₉H₆₃N₂O₂ (M−H−2Cl): 471.4890. Found: 471.4886.

### Example 9C

### 2,2-Bis(dodecyloxymethyl)-N,N,N′,N′-tetramethyl-1,3-propanediaminium dichloride (9c)

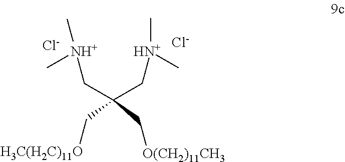

Compound 8c (Example 8C) (11.72 g, 16.9 mmol), dimethylamine in THF (2M, 85 mL, 0.17 mol, 10 eq), potassium carbonate (5.83 g, 42.2 mmol), and THF (10 mL) are heated and stirred at 160-170° C. in a sealed tube following the procedure of Example 9A. The reaction mixture is filtered and the filtrate is concentrated at 35˜40° C. to give a yellow crystalline solid, 1,3-bis(dimethylamino)-2,2-bis(dodecyloxymethyl)propane. The crude solid is taken up in dichloromethane (50 mL) and ice cold 2 M HCl (50 mL) is added to give an off white crystalline solid. The title compound is recrystallized from ethyl acetate acetone to give colourless rectangular crystals: yield 6.90 g, 68.2%; mp 128-130° C.; R_{F} 0.34 on basic alumina (hexanes, ethyl acetate, methanol 96:4:0.4); ¹H NMR δ 0.88 ppm (t, 6H, J=6.9 Hz, 2×Me), 1.26-1.31 (br s, 36H, 18×CH₂), 1.55 (pentet, 4H, J=6.5 Hz, 2 OCH₂CH₂), 2.97 (s, 12H, 2×N(CH₃)₂), 3.47 (t, 4H, J=6.6 Hz, dodecyl OCH₂), 3.67 (s, 4H, OCH₂C), 3.78 (s, 4H, CH₂N), 11.69 (brs, HN); ¹³C NMR δ 71.7 (CH₂CH₂OC), 66.9 (CCH₂OCH₂C), 58.6 (CH₂N), 47.6 (N(CH₃)₂), 44.5 (q C), 31.9 (CH₂CH₂CH₃), 29.7, 29.6, 29.6, 29.6, 29.5, 29.4, 29.3 (dodecyl CH₂), 26.2 (CH₂CH₂CH₂O), 22.7 (CH₂CH₃), 14.1 (Me); HR ESI MS: m/z calc for C₃₃H₇₁N₂O₂ (M−H−2Cl): 527.5516. found 527.5517.

### Example 9D

### N,N,N′,N′-Tetramethyl-2,2-bis(tetradecyloxymethyl)-1,3-propanediaminium dichloride (9d)

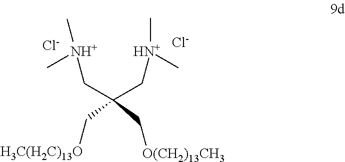

A mixture of compound 8d (Example 8D) (15.24 g, 20.37 mmol), dimethyl amine in THF (2M, 50.9 mL, 102 mmol, 5.0 eq), and potassium carbonate (7.03 g, 50.93 mmol) are heated in a sealed tube following the procedure of Example 9A, except that the same amount of dimethylamine was added after 48 h, to give an off white crystalline solid, 1,3-bis(dimethylamino)-2,2-bis(tetradecyloxymethyl)propane, that is taken up in dichloromethane (50 mL). The resulting solution is shaken with ice cold 2 M HCl (50 mL). The aqueous layer is extracted with dichloromethane (3×50 mL), then the combined organic layers are washed with water (50 mL), dried (MgSO₄), and concentrated to give the title compound (9d) as a colourless crystalline solid, that is recrystallized from ethyl acetate to give colourless crystals: yield 9.97 g, 74.8%; mp 129-130° C.; R_{F} 0.36 on basic alumina (hexanes, ethyl acetate, methanol 96:4:0.4); ¹H NMR δ 0.88 ppm (t, 6H, J=6.9 Hz, 2×Me), 1.26-1.31 (br s, 44H, 22×CH₂), 1.55 (pentet, 4H, J=6.5 Hz, 2 OCH₂CH₂), 2.97 (s, 6H, 2×N(CH₃)₂, 3.47 (t, 4H, J=6.6 Hz, tetradecyl OCH₂), 3.67 (s, 4H, OCH₂C), 3.82 (s, 4H, CH₂N), 11.88 (brs, HN); ¹³C NMR δ 71.9 (CH₂CH₂OC), 66.9 (CCH₂OCH₂C), 58.6 (CH₂N), 47.7 (N(CH₃)₂), 44.7 (q C), 32.1 (CH₂CH₂CH₃), 29.8, 29.6, 29.5, 29.5, (tetradecyl CH₂), 26.4 (CH₂CH₂CH₂O), 22.8 (CH₂CH₃), 14.3 (Me); HR ESI MS m/z calc for C₃₇H₇₉N₂O₂(M−2Cl−H): 583.6142. Found: 583.6139.

### Example 10A

### N,N,N,N′,N′,N′-hexamethyl-2,2-bis(octyloxymethyl)-1,3-propanediammonium diiodide (10a)

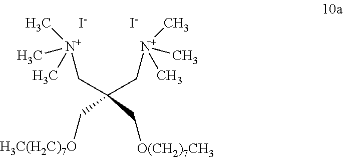

An aqueous NaOH solution (2 M, 30 mL) is added to salt 9a (Example 9A) (4.44 g, 9.1 mmol) and the resulting mixture is extracted with dichloromethane (3×50 mL). The combined extracts are washed with water and dried (MgSO₄) and concentrated to give a colourless syrup, N,N,N′,N′-tetramethyl-2,2-bis(octyloxymethyl)-1,3-propanediamine: yield 2.45 g, 64.8%; R_{F} 0.39 on basic alumina (hexanes, ethyl acetate, methanol 96:4:0.4); ¹H NMR δ 0.88 ppm (t, 6H, J=6.9 Hz, 2×Me), 1.26-1.32 (br s, 20H, 10×CH₂), 1.54 (pentet, 4H, J=7.0 Hz, 2 OCH₂CH₂), 2.26 (s, 12H, 2×N(CH₃)₂), 3.26 (s, 4H, 2×NCH₂), 3.26 (s, 4H, OCH₂C), 3.33 (t, J=6.5 Hz, 4H, octyl OCH₂); ¹³C NMR δ 71.2 (CH₂CH₂OC), 70.8 (CCH₂OCH₂C), 59.9 (CH₂N), 48.7 (N(CH₃)₂), 45.8 (q C), 32.0 (CH₂CH₂CH₃), 29.9, 29.6, 29.5, (3 octyl CH₂), 26.5 (CH₂CH₂CH₂O), 22.8 (CH₂CH₃), 14.2 (Me).

Methyl iodide (3.6 mL, 57.9 mmol, 10.0 eq) is added to a stirred solution of N,N,N′,N′-tetramethyl-2,2-bis(octyloxymethyl)-1,3-propanediamine (2.4 g, 5.79 mmol) in dry THF (15 mL) and the resulting solution is refluxed for 24 h, then concentrated. The title compound (10a), a light yellow crystalline solid, is recrystallized from ethyl acetate and acetone to give colourless crystals: yield 3.01 g, 74.5%; mp 160-162° C.; R_{F} on basic alumina 0.53 (chloroform acetone methanol 211); ¹H NMR δ 0.88 ppm (t, 6H, J=6.9 Hz, 2×Me), 1.27-1.29 (br s, 20H, 10×CH₂), 1.60 (pentet, 4H, J=6.6 Hz, 2 OCH₂CH₂), 3.51 (t, 4H, J=6.7 Hz, octyl OCH₂), 3.65 (s, 18H, 6×CH₃), 3.91 (s, 4H, OCH₂C), 4.45 (s, 4H, CH₂N); ¹³C NMR δ 72.2 (CH₂CH₂OC), 68.2 (CCH₂OCH₂C), 67.7 (CH₂N), 56.5 (N(CH₃)₃), 49.1 (q C), 31.9 (CH₂CH₂CH₃), 29.7, 29.4, 29.3, (octyl CH₂), 26.4 (CH₂CH₂CH₂O), 22.7 (CH₂CH₃), 14.2 (Me). HR ESI MS m/z calc for C₂₇H₆₀NIO₂ (M−I): 571.3700. found: 571.3702; calc. for C₅₄H₁₂₀N₄I₃O₄ (2M−I): 1269.6444. found: 1269.6443.

### Example 10B

### 2,2-Bis(decyloxymethyl)-N,N,N,N′,N′,N′-hexamethyl-1,3-propanediammonium diiodide (10b)

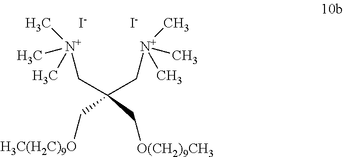

An aqueous NaOH solution (2 M, 20 mL) is added to salt 9b (Example 9B) (2.5 g, 4.37 mmol) then extracted with dichloromethane (3×25 mL) following the procedure of Example 10A to yield a colourless syrup of 2,2-bis(decyloxymethyl)-N,N,N′,N′-tetramethyl-1,3-propanediamine, yield: 1.7 g, 79%; R_{F} 0.42 on basic alumina (hexanes, ethyl acetate, methanol 96:4:0.4); ¹H NMR δ 0.88 ppm (t, 6H, J=6.9 Hz, 2×Me), 1.26-1.28 (br s, 28H, 14×CH₂), 1.54 (br m, 4H, 2 OCH₂CH₂), 2.26 (s, 16H, 2×N(CH₃)₂, 2×NCH₂), 3.26 (s, 4H, OCH₂C), 3.38 (t, J=6.5 Hz, 4H, decyl OCH₂); ¹³C NMR δ 71.1 (CH₂CH₂OC), 70.7 (CCH₂OCH₂C), 59.9 (CH₂N), 48.6 (N(CH₃)₂), 45.6 (q C), 32.1 (CH₂CH₂CH₃), 29.9, 29.8, 29.8, 29.7, 29.5, (decyl CH₂), 26.5 (CH₂CH₂CH₂O), 22.8 (CH₂CH₃), 14.2 (Me).

Methyl iodide (2.24 mL, 36.1 mmol, 10 eq) is added to a stirred solution of 2,2-bis(decyloxymethyl)-N,N,N′,N′-tetramethyl-1,3-propanediamine (1.7 g, 3.6 mmol) in dry THF (25 mL) following the procedure of Example 10A to give the title compound (10b) as a light yellow crystalline solid that is recrystallized from ether and acetone to give colourless crystals: yield 2.4 g, 73%; mp 75° C.; R_{F} on basic alumina 0.57 (chloroform acetone methanol 2 1 1); ¹H NMR δ 0.88 ppm (t, 6H, J=6.9 Hz, 2×Me), 1.26-1.30 (br s, 28H, 14×CH₂), 1.60 (pentet, 4H, J=6.5 Hz, 2 OCH₂CH₂), 3.51 (t, 4H, J=6.9 Hz, decyl OCH₂), 3.65 (s, 18H, 6×CH₃), 3.91 (s, 4H, OCH₂C), 4.44 (s, 4H, CH₂N); ¹³C NMR δ 71.9 (CH₂CH₂OC), 68.2 (CCH₂OCH₂C), 67.9 (CH₂N), 56.1 (N(CH₃)₃), 48.7 (q C), 31.7 (CH₂CH₂CH₃), 29.45, 29.43, 29.37, 29.14 (4 decyl CH₂), 29.25 (OCH₂CH₂), 26.2 (CH₂CH₂CH₂O), 22.5 (CH₂CH₃), 14.0 (Me); HR ESI-MS m/z calc. for C₃₁H₆₈N₂IO₂⁺627.4326 (M−I). found 627.4326; calc. for C₆₂H₁₃₆I₃N₄O₄⁺1381.7696 (2M−I). found 1381.7694.

### Example 10C

### 2,2-Bis(dodecyloxymethyl)-N,N,N,N′,N′,N′-hexamethyl-1,3-propanediammonium diiodide (10c)

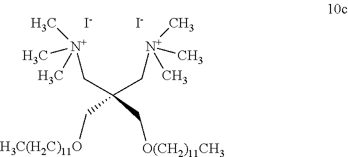

An aqueous NaOH solution (2 M, 50 mL) is added to salt 9c (Example 9C) (4.75 g, 7.9 mmol) and the resulting solution is extracted with dichloromethane following the procedure of Example 10A to yield a colourless syrup, 2,2-bis(dodecyloxymethyl)-N,N,N′N′-tetramethyl-1,3-propanediamine: yield 3.27 g, 78.4%; R_{F} 0.48 on basic alumina (hexanes, ethyl acetate, methanol 96:4:0.4); ¹H NMR δ 0.88 ppm (t, 6H, J=6.9 Hz, 2×Me), 1.26-1.33 (br s, 36H, 18×CH₂), 1.53 (pentet, 4H, J=7.0 Hz, 2 OCH₂CH₂), 2.26 (2s, 16H, 2×N(CH₃)₂, 2×NCH₂), 3.26 (s, 4H, OCH₂C), 3.33 (t, J=6.5 Hz, 4H, dodedecyl OCH₂); ¹³C NMR δ 71.2 (CH₂CH₂OC), 70.7 (CCH₂OCH₂C), 59.9 (CH₂N), 48.7 (N(CH₃)₂), 45.7 (q C), 32.1 (CH₂CH₂CH₃), 29.9, 29.8, 29.8, 29.7, 29.5 (dodecyl CH₂), 26.5 (CH₂CH₂CH₂O), 22.8 (CH₂CH₃), 14.3 (Me).

Methyl iodide (3.9 mL, 61.7 mmol, 10 eq) is added to a stirred solution of 2,2-bis(dodecyloxymethyl)-N,N,N′,N′-tetramethyl-1,3-propanediamine (3.25 g, 6.18 mmol) in dry THF (20 mL) following the procedure of Example 10A to give compound 10c as a light yellow crystalline solid that is recrystallized from ethyl acetate and acetone to give colourless crystals: yield 3.75 g, 75.5%; R_{F} on basic alumina 0.48 (hexanes, ethyl acetate, methanol 96:4:0.4); mp 130-132° C.; ¹H NMR δ 0.88 ppm (t, 6H, J=6.9 Hz, 2×Me), 1.26-1.30 (br s, 36H, 18×CH₂), 1.59 (pentet, 4H, J=6.4 Hz, 2 OCH₂CH₂), 3.51 (t, 4H, J=6.7 Hz, dodecyl OCH₂), 3.65 (s, 18H, 6×CH₃), 3.91 (s, 4H, OCH₂C), 4.45 (s, 4H, CH₂N); ¹³C NMR δ 72.1 (CH₂CH₂OC), 68.2 (CCH₂OCH₂C), 67.9 (CH₂N), 56.4 (N(CH₃)₃), 48.9 (q C), 31.9 (CH₂CH₂CH₃), 29.6, 29.5, 29.4, 29.4, (dodecyl CH₂), 26.3 (CH₂CH₂CH₂O), 22.7 (CH₂CH₃), 14.1 (Me); LR ESI MS m/z calc for C₃₅H₇₆N₂IO₂ 683.49. found 683.3 (M−I). Anal. Calc. for C35H76N2O2I2: C, 51.85; H, 9.45; N, 3.46. Found: C, 51.43; H, 9.32; N, 3.71.

### Example 10D

### N,N,N,N′,N′,N′-Hexamethyl-2,2-bis(tetradecyloxymethyl)-1,3-propanediammonium diiodide (10d)

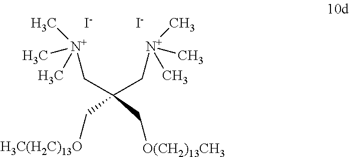

An aqueous NaOH solution (2 M, 40 mL) is added to salt 9d (Example 9D) (9.97 g, 15.2 mmol) and the resulting solution is extracted with dichloromethane (3×50 mL). The combined extracts are washed with water and dried (MgSO₄) and concentrated to colourless syrup, N,N,N′,N′-tetramethyl-2,2-bis(tetradecyloxymethyl)-1,3-propanediamine: yield 7.85 g, 65.9%; R_{F} 0.51 on basic alumina (hexanes, ethyl acetate, methanol 96:4:0.4); ¹H NMR δ 0.88 ppm (t, 6H, J=6.9 Hz, 2×Me), 1.26-1.31 (br s, 44H, 22×CH₂), 1.51 (pentet, 4H, J=7.0 Hz, 2 OCH₂CH₂), 2.26 (s, 16H, 2×N(CH₃)₂, 2×NCH₂), 3.26 (s, 4H, OCH₂C), 3.33 (t, J=6.5 Hz, 4H, tetradecyl OCH₂); ¹³C NMR δ 71.2 (CH₂CH₂OC), 70.7 (CCH₂OCH₂C), 59.9 (CH₂N), 48.7 (N(CH₃)₂), 45.7 (q C), 32.1 (CH₂CH₂CH₃), 29.91, 29.86, 29.85, 29.82, 29.81, 29.80, 29.65 (8 tetradecyl CH₂), 29.52 (OCH₂CH₂), 26.5 (CH₂CH₂CH₂O), 22.8 (CH₂CH₃), 14.3 (Me); LR ESI MS: m/z calc for C₃₇H₇₉N₂O₂ 583.61. Found 583.5.

Methyl iodide (4.3 mL, 68.7 mmol, 10 eq) is reacted with N,N,N′,N′-tetramethyl-2,2-bis(tetradecyloxymethyl)-1,3-propanediamine (4.0 g, 6.87 mmol) in dry THF (20 mL) following the procedure of Example 10A to give the title compound as a light yellow crystalline solid that is recrystallized from ethyl acetate and acetone to give colourless crystals: yield 5.7 g, 96%; R_{F} on basic alumina 0.63 (chloroform:acetone:methanol 2:1:1); mp 127-128° C.; ¹H NMR δ 0.88 ppm (t, 6H, J=7.0 Hz, 2×Me), 1.26-1.30 (br s, 44H, 22×CH₂), 1.57 (pentet, 4H, J=7.1 Hz, 2 OCH₂CH₂), 3.50 (t, 4H, J=6.8 Hz, tetradecyl OCH₂), 3.63 (s, 18H, 6×CH₃), 3.93 (s, 4H, OCH₂C), 4.48 (s, 4H, CH₂N); ¹³C NMR δ 72.2 (CH₂CH₂OC), 68.2 (CCH₂OCH₂C), 67.7 (CH₂N), 56.6 (N(CH₃)₃), 49.1 (q C), 32.0 (CH₂CH₂CH₃), 29.83, 29.80, 29.78, 29.78, 29.76, 29.54, 29.48 (8 tetradecyl CH₂), 29.68 (OCH₂CH₂), 26.5 (CH₂CH₂CH₂O), 22.8 (CH₂CH₃), 14.23 (Me); LR ESI MS m/z calc for C₃₉H₈₄N₂O₂I (M−I) 739.56. found 739.3. Anal. Calc. for C₃₉H₈₄N₂O₂I₂: C, 54.03; H, 9.77; N, 3.23. Found: C, 53.78; H, 9.76; N, 3.09.

### Example 11A

### 1,3-Bis(1-azacyclopentyl)-2,2-bis(octyloxymethyl)propane dihydrochloride (11a)

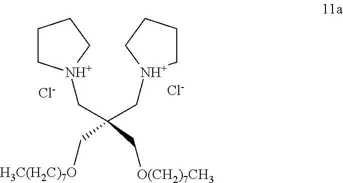

A stirred solution of compound 8a (Example 8A) (18.0 g, 31.0 mmol) in pyrrolidine (100 mL) containing potassium carbonate (10.7 g, 31.0 mmol, 2.5 eq) is refluxed under nitrogen for 48 h, allowed to cool to rt, then filtered. The solid is washed with dichloromethane (2×10 mL) and the filtrate and washings are combined and diluted with dichloromethane (100 mL). The resulting solution is washed with water (3×100 mL), dried (MgSO₄) and concentrated at 30° C. to give crude 1,3-bis(1-azacyclopentyl)-2,2-bis(octyloxymethyl)propane: yield 14.20 g. This product is taken up in dichloromethane (100 mL) and the resulting solution is shaken with ice cold 2 M HCl (100 mL). The aqueous layer is extracted with dichloromethane (2×100 mL), then the combined organic layers are washed with water (20 mL), dried (MgSO₄) and concentrated to give the title compound as a light yellow crystalline solid that is recrystallized from hexanes, ethyl acetate 2:1 to give colorless crystals: yield 12.5 g, 75%; mp 124-125° C., R_{F} 0.22 on basic alumina (hexanes, ethyl acetate, methanol, 96:4:0.4); ¹H NMR δ 0.88 ppm (t, 6H, J=6.9 Hz, 2×Me), 1.26-1.31 (br s, 20H, 10×CH₂), 1.55 (pentet, 4H, J=6.6 Hz, 2 OCH₂CH₂), 2.06 (YY′ part of AA′BB′XX′YY′ pattern, 4H, 1/2 of 2 N(CH₂CH₂)₂), 2.25 (XX′ part of AA′BB′XX′YY′ pattern, 4H, 1/2 of 2 N(CH₂CH₂)₂), 3.21 (BB′ part of AA′BB′XX′YY′ pattern, 4H, 1/2 of 2 N(CH₂)), 3.46 (t, 4H, J=6.6 Hz, octyl OCH₂), 3.59 (s, 4H, OCH₂C), 3.84 (s, 4H, CH₂N), 3.91 (AA′ part of AA′BB′XX′YY′ pattern, 4H, 1/2 of 2 N(CH₂)), 11.51 (br s, HN); ¹³C NMR δ 71.5 (CH₂CH₂OC), 67.5 (CCH₂OCH₂CH₂), 57.8 (NCH₂), 56.9 (CH₂N), 44.3 (q C), 31.7 (CH₂CH₂CH₃), 29.41, 29.25, 29.18 (3 octyl CH₂), 26.2 (CH₂CH₂CH₂O), 23.5 (NCH₂CH₂), 22.6 (CH₂CH₃), 14.0 (Me); HR ESI MS m/z calcd for C₂₉H₅₉N₂O₂ (M−H−2Cl) 467.4577. found 467.4578.

### Example 11B

### 1,3-Bis(1-azacyclopentyl)-2,2-bis(decyloxymethyl)propane dihydrochloride (11b)

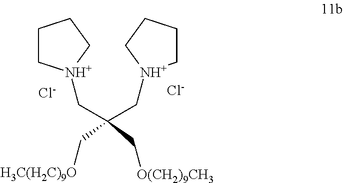

Treatment of a solution of compound 8b (Example 8B) (16.0 g, 25.0 mmol) in pyrrolidine (100 mL) containing potassium carbonate (8.69 g, 62.8 mmol, 2.5 eq) following the procedure of Example 11A gives a light brown syrup, 1,3-bis(1-azacyclopentyl)-2,2-bis(decyloxymethyl)propane: yield 11.93 g. Addition of ice cold 2 M HCl (100 mL) gives a light yellow crystalline solid that is recrystallized from ethyl acetate to give colorless crystals: yield 10.9 g, 76%; mp 125-126° C., R_{F} 0.24 on basic alumina (hexanes, ethyl acetate, methanol, 96:4:0.4); ¹H NMR δ 0.88 ppm (t, 6H, J=6.8 Hz, 2×Me), 1.26-1.28 (br s, 28H, 14×CH₂), 1.55 (pentet, 4H, J=6.3 Hz, 2 OCH₂CH₂), 2.05 (YY′ part of AA′BB′XX′YY′ pattern, 4H, 1/2 of 2 2 N(CH₂CH₂)₂), 2.25 (XX′ part of AA′BB′XX′YY′ pattern, 4H, 1/2 of 2 2 N(CH₂CH₂)₂), 3.21 (BB′ part of AA′BB′XX′YY′ pattern, 4H, N(CH₂)₂), 3.45 (t, 4H, J=6.6 Hz, decyl OCH₂), 3.58 (s, 4H, OCH₂C), 3.84 (s, 4H, CH₂N), 3.91 (BB′ part of AA′BB′XX′YY′ pattern, 4H, N(CH₂)₂), 11.5 (br s, HN); ¹³C NMR δ 71.7 (CH₂CH₂OC), 67.5 (CCH₂O), 57.9 (NCH₂), 57.0 ((CH₂)₂N), 44.5 (q C), 32.0 (CH₂CH₂CH₃), 29.71, 29.66, 29.59, 29.47, 29.40 (6 decyl CH₂), 26.3 (CH₂CH₂CH₂O), 23.6 (N(CH₂CH₂)₂), 22.8 (CH₂CH₃), 14.2 (Me); HR ESI MS m/z calcd for C₃₃H₆₇N₂O₂ (M−H−2Cl) 523.5203. found 523.5204.

### Example 11C

### 1,3-Bis(1-azacyclopentyl)-2,2-bis(dodecyloxymethyl)propane dihydrochloride (11c)

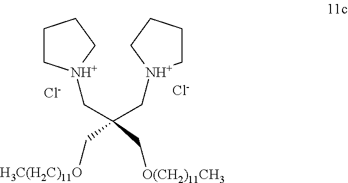

Treatment of a solution of compound 8c (Example 8C) (12.2 g, 17.6 mmol) in pyrrolidine (100 mL) containing potassium carbonate (6.1 g, 44 mmol, 2.5 eq) following the procedure of Example 11A gives an orange syrup, 1,3-bis(1-azacyclopentyl)-2,2-bis(dodecyloxymethyl)propane: yield 12.9 g. Addition of ice cold 2 M HCl (100 mL) and dichloromethane (30 mL) provided a light pink crystalline solid that is dissolved in dichloromethane (30 mL). The dichloromethane solution is washed with distilled water (10 mL), dried (MgSO₄) and concentrated to a crystalline solid that is recrystallized from ethyl acetate to give colorless crystals: yield 8.8 g, 77%; mp 127° C.; R_{F} 0.28 on basic alumina (hexanes, ethyl acetate, methanol, 96:4:0.4); ¹H NMR δ 0.88 ppm (t, 6H, J=6.9 Hz, 2×Me), 1.26-1.28 (br s, 36H, 18×CH₂), 1.55 (pentet, 4H, J=6.4 Hz, 2 OCH₂CH₂), 2.06 (YY′ part of AA′BB′XX′YY′ pattern, 4H, 1/2 of 2 2 N(CH₂CH₂)₂), 2.25 (XX′ part of AA′BB′XX′YY′ pattern, 4H, 1/2 of 2 2 N(CH₂CH₂)₂), 3.21 (BB′ part of AA′BB′XX′YY′ pattern, 4H, N(CH₂)₂), 3.45 (t, 4H, J=6.5 Hz, dodecyl OCH₂), 3.58 (s, 4H, OCH₂C), 3.83 (s, 4H, CH₂N), 3.91 (AA′ part of AA′BB′XX′YY′ pattern, 4H, N(CH₂)₂), 11.5 (br s, HN); ¹³C NMR δ 71.7 (CH₂CH₂OC), 67.6 (CCH₂O), 58.0 (NCH₂), 57.1 ((CH₂)₂N), 44.5 (q C), 32.0 (CH₂CH₂CH₃), 29.7, 29.6, 29.5, 29.4, (dodecyl CH₂), 26.3 (CH₂CH₂CH₂O), 23.6 (N(CH₂CH₂)₂), 22.8 (CH₂CH₃), 14.2 (Me); HR ESI MS ink calcd for C₃₇H₇₅N₂O₂ (M−H−2Cl) 579.5829. found 579.5826.

### Example 11D

### 1,3-Bis(1-azacyclopentyl)-2,2-bis(tetradecyloxymethyl)propane dihydrochloride (11d)

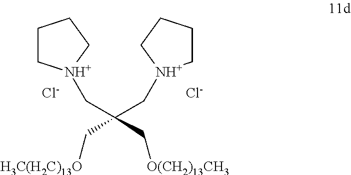

Treatment of a solution of compound 8d (Example 8D) (14.96 g, 31.29 mmol) in pyrrolidine (100 mL) containing potassium carbonate (10.78 g, 78.0 mmol, 2.5 eq) following the procedure of Example 11A gives a brown solid, 1,3-bis(1-azacyclopentyl)-2,2-bis(tetradecyloxymethyl)propane, yield 17.26 g. Addition of ice cold 2 M HCl (100 mL) and dichloromethane (30 mL) provided a yellow crystalline solid (18.11 g) that is dissolved in dichloromethane (30 mL). The solution is dried (MgSO₄) and concentrated to colorless crystalline solid that is recrystallized from ethyl acetate to give the title compound as colorless crystals: yield 17.5 g, 79%; mp 128° C.; R_{F} 0.33 on basic alumina (hexanes, ethyl acetate, methanol, 96:4:0.4); ¹H NMR δ 0.88 ppm (t, 6H, J=6.9 Hz, 2×Me), 1.26-1.28 (br s, 44H, 22×CH₂), 1.55 (pentet, 4H, J=6.3 Hz, 2 OCH₂CH₂), 2.05 (YY′ part of AA′BB′XX′YY′ pattern, 4H, 1/2 of 2 2 N(CH₂CH₂)₂), 2.25 (XX′ part of AA′BB′XX′YY′ pattern, 4H, 1/2 of 2 2 N(CH₂CH₂)₂), 3.21 (BB′ part of AA′BB′XX′YY′ pattern, 4H, N(CH₂)₂), 3.45 (t, 4H, J=6.5 Hz, tetradecyl OCH₂), 3.58 (s, 4H, OCH₂C), 3.84 (s, 4H, CH₂N), 3.91 (AA′ part of AA′BB′XX′YY′ pattern, 4H, N(CH₂)₂), 11.5 (br s, HN); ¹³C NMR δ 71.7 (CH₂CH₂OC), 67.6 (CCH₂O), 58.0 (NCH₂), 57.1 ((CH₂)₂)N), 44.6 (q C), 32.1 (CH₂CH₂CH₃), 29.83, 29.82, 29.79, 29.65, 29.57, 29.49 (8 tetradecyl CH₂), 26.4 (CH₂CH₂CH₂O), 23.7 (N(CH₂CH₂)₂), 22.8 (CH₂CH₃), 14.2 (Me); HR ESI MS m/z calcd for C₄₁H₈₃N₂O₂ (M−H−2Cl) 635.6455. found 635.6454.

### Example 12A

### 1,3-bis(1-methyl-1-azoniacyclopentyl)-2,2-bis(octyloxymethyl)propane diiodide (12a)

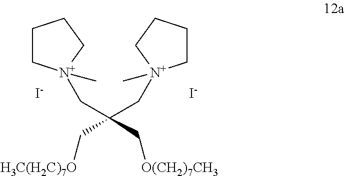

An aqueous 2 M NaOH solution (50 mL) is added to compound 11a (Example 11A) (12.5 g, 23.2 mmol) and the resulting mixture is extracted with dichloromethane (3×50 mL). The combined extracts are washed with water and dried (MgSO₄), and concentrated to give the free base, 1,3-bis(1-azacyclopentyl)-2,2-bis(octyloxymethyl)propane as a light yellow syrup: yield 10.3 g, 72%; R_{F} 0.32 on basic alumina (hexanes, ethyl acetate, methanol, 96:4:0.4); ¹H NMR δ 0.88 ppm (t, 6H, J=6.8 Hz, 2×Me), 1.27-1.34 (br s, 20H, 10×CH₂), 1.53 (pentet, 4H, J=6.7 Hz, 2 OCH₂CH₂), 1.68 (m, 8H, 2NCH₂CH₂), 2.48 (s, 4H CH₂N), 2.56 (m, 8H, 2N(CH₂)₂, 3.27 (s, 4H, OCH₂C), 3.32 (t, 4H, J=6.4 Hz, octyl OCH₂); ¹³C NMR δ 71.7 (CCH₂OCH₂CH₂), 71.1 (CH₂CH₂OC), 56.81 (CH₂N), 56.84 (NCH₂), 45.7 (q C), 31.9 (CH₂CH₂CH₃), 29.87, 29.55, 29.43 (3 octyl CH₂), 26.4 (CH₂CH₂CH₂O), 24.3 (NCH₂CH₂), 22.8 (CH₂CH₃), 14.1 (Me).

Methyl iodide (13.3 g, 21.3 mmol, 10 eq) is added to a stirred solution of 1,3-bis(1-azacyclopentyl)-2,2-bis(octyloxymethyl)propane (9.90 g, 21.3 mmol) in dry THF (50 mL). The resulting mixture is refluxed under nitrogen for 48 h, then concentrated. The title compound, a light brown crystalline solid, is recrystallized from ethyl acetate to give colorless crystals: yield 13 g, 81%; mp 92° C.; R_{F} 0.69 on basic alumina (chloroform, acetone, methanol, ammonia 2:2:1:0.5); ¹H NMR δ 0.89 ppm (t, 6H, J=6.9 Hz, 2×Me), 1.26-1.30 (br s, 20H, 10×CH₂), 1.57 (pentet, 4H, J=6.5 Hz, 2 OCH₂CH₂), 2.18 (YY′ part of AA′BB′XX′YY′ pattern, 4H, 1/2 of 2 2 N(CH₂CH₂)₂), 2.38 (XX′ part of AA′BB′XX′YY′ pattern, 4H, 1/2 of 2 2 N(CH₂CH₂)₂)), 3.46 (s, 6H, 2 NCH₃), 3.49 (t, 4H, J=6.6 Hz, octyl OCH₂), 3.96 (s, 4H, OCH₂C), 4.04 (m, 8H, 2 N(CH₂)₂), 4.63 (s, 4H, 2 CH₂N); ¹³C NMR δ 71.9 (CH₂CH₂OC), 68.3 (CCH₂OCH₂CH₂), 66.9 (NCH₂), 65.4 (CH₂N), 48.5 (CH₃N), 48.4 (q C), 31.8 (CH₂CH₂CH₃), 29.5, 29.3, 29.2 (3 octylCH₂), 26.3 (CH₂CH₂CH₂O), 22.6 (CH₂CH₃), 21.1 (NCH₂CH₂), 14.1 (Me); HR ESI-MS m/z calcd for C₃₁H₆₄IN₂O₂ 623.4013 (M−I). found 623.4011.

### Example 12B

### 2,2-Bis(decyloxymethyl)-1,3-bis(1-methyl-1-azoniacyclopentyl)propane diiodide (12b)

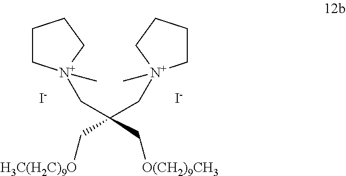

Aqueous 2 M NaOH solution (50 mL) and compound 11b (Example 11B) (10.9 g, 18.3 mmol) are reacted following the procedure of Example 12A to give a colorless syrup, 1,3-bis(1-azacyclopentyl)-2,2-bis(decyloxymethyl)propane: yield 9.50 g, 72.5%; R_{F} 0.34 on basic alumina (hexanes, ethyl acetate, methanol, 96:4:0.4); ¹H NMR δ 0.88 ppm (t, 6H, J=6.9 Hz, 2×Me), 1.26-1.31 (br s, 28H, 14×CH₂), 1.53 (pentet, 4H, J=Hz, 2 OCH₂CH₂), 1.68 (m, 8H, 2 N(CH₂CH₂)), 2.49 (s, 4H, NCH₂C, 2.57 (m, 8H, 2 N(CH₂)₂), 3.29 (s, 4H, OCH₂C), 3.33 (t, 4H, J=6.5 Hz, decyl OCH₂); ¹³C NMR δ 71.9 (CCH₂OCH₂CH₂), 71.3 (CH₂CH₂OC), 57.2 (CH₂N), 57.0 (N(CH₂)₂), 45.8 (q C), 32.1 (CH₂CH₂CH₃), 29.93, 29.85, 29.78 29.68, 29.52 (6 decyl CH₂), 26.5 (CH₂CH₂CH₂O), 24.4 (N(CH₂CH₂)₂), 22.8 (CH₂CH₃), 14.3 (Me).

Methyl iodide (16.6 g, 117 mmol, 10 eq) and 1,3-bis(1-azacyclopentyl)-2,2-bis(decyloxymethyl)propane (6.1 g, 11.6 mmol) in dry THF (50 mL) are treated following the procedure of Example 12A to give a light brown crystalline solid that is recrystallized from ethyl acetate to give an off white crystalline solid: yield 6.0 g, 81%; mp 95-96° C.; R_{F} 0.71 on basic alumina (chloroform, acetone, methanol, ammonia 2:2:1:0.5); ¹H NMR δ 0.88 ppm (t, 6H, J=6.9 Hz, 2×Me), 1.26-1.30 (br s, 28H, 14×CH₂), 1.57 (pentet, 4H, J=6.5 Hz, 2 OCH₂CH₂), 2.18 (YY′ part of AA′BB′XX′YY′ pattern, 4H, 1/2 of 2 2 N(CH₂CH₂)₂), 2.38 (XX′ part of AA′BB′XX′YY′ pattern, 4H, 1/2 of 2 2 N(CH₂CH₂)₂)), 3.46 (s, 6H, 2 NCH₃), 3.49 (t, 4H, J=6.6 Hz, decyl OCH₂), 3.96 (s, 4H, OCH₂C), 4.04 (m, 8H, 2N(CH₂)₂), 4.63 (s, 4H, 2 CH₂N); ¹³C NMR δ 72.1 (CH₂CH₂OC), 68.2 (CCH₂OCH₂CH₂), 66.9 (NCH₂), 65.2 (CH₂N), 48.6 (CH₃N), 48.3 (q C), 32.0 (CH₂CH₂CH₃), 29.71, 29.67, 29.66, 29.47, 29.39 (5 decyl CH₂), 26.5 (CH₂CH₂CH₂O), 22.8 (CH₂CH₃), 21.2 (NCH₂CH₂), 14.2 (Me); LR ESI MS m/z calcd for C₃₅H₇₂IN₂O₂(M−I) 679.64. found 679.3, calcd for (M−2I) 276.3. found 276.3. Anal. Calcd. for C₃₅H₇₂I₂N₂O₂: C, 52.11; H, 9.00; N, 3.47. Found: C, 52.08; H, 9.17; N, 3.69.

### Example 12C

### 2,2-Bis(dodecyloxymethyl)-1,3-bis(1-methyl-1-azoniacyclopentyl)propane diiodide (12c)

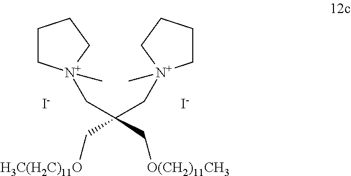

Reaction of 2 M NaOH (50 mL) with salt 11c (Example 11C) (8.8 g, 13.5 mmol) following the procedure of Example 12A gives 1,3-bis(1-azacyclopentyl)-2,2-bis(dodecyloxymethyl)propane as a colorless syrup: yield 7.5 g, 74%; R_{F} 0.36 on basic alumina (hexanes, ethyl acetate, methanol, 96:4:0.4); ¹H NMR δ 0.88 ppm (t, 6H, J=6.9 Hz, 2×Me), 1.26-1.31 (br s, 36H, 18×CH₂), 1.53 (pentet, 4H, J=7.0 Hz, 2 OCH₂CH₂), 1.69 (m, 8H, 2N(CH₂CH₂)₂), 2.49 (s, 4H, CH₂N), 2.57 (m, 8H, 2 N(CH₂)₂), 3.28 (s, 4H, OCH₂C), 3.33 (t, 4H, J=6.5 Hz, dodecyl OCH₂); ¹³C NMR δ 71.9 (CCH₂O), 71.3 (CH₂CH₂OC), 57.3 (CH₂N), 57.0 (N(CH₂)₂), 45.8 (q C), 32.0 (CH₂CH₂CH₃), 29.3, 29.8, 29.7 (dodecyl CH₂), 26.4 (CH₂CH₂CH₂O), 24.2 (N(CH₂CH₂)₂), 22.9 (CH₂CH₃), 14.3 (Me).

Methyl iodide (8 mL, 129.7 mmol, 10 eq) is reacted with 1,3-bis(1-azacyclopentyl)-2,2-bis(dodecyloxymethyl)propane (7.5 g, 13 mmol) in dry THF (50 mL) following the procedure of Example 12A to give the title compound, as an off white crystalline solid, that is recrystallized from ethyl acetate to give colorless crystals: yield 7.5 g, 84%; mp 100-101° C.; R_{F} 0.74 on basic alumina (chloroform, acetone, methanol, ammonia 2:2:1:0.5); ¹H NMR δ 0.88 ppm (t, 6H, J=6.9 Hz, 2×Me), 1.25-1.23 (br s, 36H, 18×CH₂), 1.57 (pentet, 4H, J=6.6 Hz, 2 OCH₂CH₂), 2.17 (YY′ part of AA′BB′XX′YY′ pattern, 4H, 1/2 of 2 2 N(CH₂CH₂)₂), 2.38 (XX′ part of AA′BB′XX′YY′ pattern, 4H, 1/2 of 2 2 N(CH₂CH₂)₂), 3.45 (s, 2 NCH₃), 3.49 (t, 4H, J=6.6 Hz, 2 dodecyl OCH₂), 3.96 (s, 4H, OCH₂C), 4.05 (m, 8H, NCH₂), 4.64 (s, 4H, CH₂N); ¹³C NMR δ 71.9 (CH₂CH₂OC), 68.3 (CCH₂OCH₂CH₂), 66.9 (NCH₂), 65.5 (CH₂N), 48.4 (CH₃N), 48.3 (q C), 31.8 (CH₂CH₂CH₃), 29.6, 29.5, 29.3, 29.3 (dodecyl CH₂), 26.3 (CH₂CH₂CH₂O), 22.6 (CH₂CH₃), 21.1 (NCH₂CH₂), 14.0 (Me); LR ESI MS m/z calcd for C₃₉H₈₀IN₂O₂ (M−I) 735.53. found 735.3; calcd for C₃₈H₇₇N₂O₂ (M−2I−Me) 593.60. found 593.4; calcd for (M−2I)/2 304.31. found 304.3. Anal. Calcd. for C₃₉H₈₀I₂N₂O₂: C, 54.29; H, 9.34; N, 3.47. Found: C, 54.16; H, 9.77; N, 3.38.

### Example 12D

### 1,3-Bis(1-methyl-1-azoniacyclopentyl)-2,2-bis(tetradecyloxymethyl)propane iodide (12d)

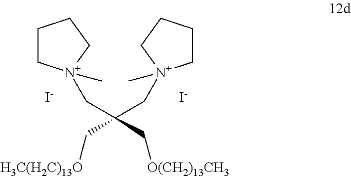

Reaction of 2 M NaOH (50 mL) with salt 11d (Example 11D) (17.5 g, 24.7 mmol) following the procedure of Example 12A gives 1,3-bis(1-azacyclopentyl)-2,2-bis(tetradecyloxymethyl)propane as a light yellow crystalline solid: yield 14.3 g, 72%; mp 100-102° C.; R_{F} 0.38 on basic alumina (hexanes, ethyl acetate, methanol, 96:4:0.4); ¹H NMR δ 0.88 ppm (t, 6H, J=6.9 Hz, 2×Me), 1.26-1.31 (br s, 44H, 22×CH₂), 1.53 (pentet, 4H, J=7.0 Hz, 2 OCH₂CH₂), 1.68 (m, 8H, 2 N(CH₂CH₂)₂), 2.49 (s, 4H, CH₂N), 2.57 (m, 8H, 2 N(CH₂)₂), 3.28 (s, 4H, OCH₂C), 3.33 (t, 4H, J=6.5 Hz, tetradecyl OCH₂); ¹³C NMR δ 71.7 (CCH₂O), 71.1 (CH₂CH₂OC), 57.1 (CH₂N), 56.9 (N(CH₂)₂), 45.6 (q C), 32.0 (CH₂CH₂CH₃), 29.80, 29.74, 29.70 29.55, 29.40 (8 tetradecyl CH₂), 26.4 (CH₂CH₂CH₂O), 24.2 (N(CH₂CH₂)₂), 22.7 (CH₂CH₃), 14.1 (Me).

Methylation of 1,3-bis(1-azacyclopentyl)-2,2-bis(tetradecyloxymethyl)propane (7.15 g, 11.3 mmol) in THF (50 mL) with methyl iodide (15.8 g, 112 mmol, 10 eq) following the procedure of Example 12A gives the title compound, a light brown crystalline solid, that is recrystallized from ethyl acetate to give an off-white crystalline solid: yield 7.6 g, 94%; mp 108° C.; R_{F} 0.76 on basic alumina (chloroform, acetone, methanol, ammonia 2:2:1:0.5); ¹H NMR δ 0.88 ppm (t, 6H, J=6.9 Hz, 2×Me), 1.26-1.29 (br s, 44H, 22×CH₂), 1.57 (pentet, 4H, J=6.5 Hz, 2 OCH₂CH₂), 2.17 (YY′ part of AA′BB′XX′YY′ pattern, 4H, 1/2 of 2 2 N(CH₂CH₂)₂), 2.38 (XX′ part of AA′BB′XX′YY′ pattern, 4H, 1/2 of 2 2 N(CH₂CH₂)₂), 3.45 (s, 2 NCH₃), 3.49 (t, 4H, J=6.6 Hz, tetradecyl OCH₂), 3.97 (s, 4H, OCH₂C), 4.05 (m, 8H, NCH₂), 4.67 (s, 4H, CH₂N); ¹³C NMR δ 72.0 (CH₂CH₂OC), 68.3 (CCH₂OCH₂CH₂), 66.9 (NCH₂), 65.3 (CH₂N), 48.6 (CH₃N), 48.3 (q C), 32.0 (CH₂CH₂CH₃), 29.72, 29.69, 29.62, 29.44, 29.39 (5 tetradecyl CH₂), 26.4 (CH₂CH₂CH₂O), 22.7 (CH₂CH₃), 21.2 (NCH₂CH₂), 14.2 (Me); LR ESI MS m/z calcd for C₄₃H₈₈IN₂O₂ (M−I) 791.59. found 791.3; calcd for M/2: 332.34. found 332.4; calcd for C₈₆H₁₇₆I₃N₄O₄ (2M−1) 1710.1. found 1709.3. Anal. Calcd for C₄₃H₈₈I₂N₂O₂.H₂O: C, 55.12; H, 9.68; N, 2.99. Found: C, 55.26; H, 9.46; N, 3.30.

### Example 13A

### 3,3-bis(octyloxymethyl)pentanedinitrile (13a)

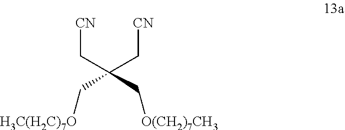

Potassium cyanide (0.80 g, 12.0 mmol, 3.0 eq) is added to a stirred solution of compound 8a (Example 8A) (2.4 g, 4.1 mmol) in dry DMF (25 mL). The resulting mixture is stirred at 80° C. for 24 h, and then allowed to cool to rt. A solid is deposited and the solution is decanted. The solid is washed with dichloromethane (2×10 mL). The combined solution and washings are concentrated to give a yellow oil that is taken up in dichloromethane (30 mL). The resulting solution is washed with water (3×25 mL), dried (MgSO₄) and concentrated to give the title compound as a light yellow oil: yield 1.3 g, 83%; R_{F} 0.42 (hexanes:ethyl acetate 9:1); ¹H NMR δ 0.88 (t, 6H, J=6.9 Hz, 2×Me), 1.23-1.33 (br s, 20H, 10×CH₂), 1.55 (pentet, 4H, J=7.0 Hz, 2 OCH₂CH₂), 2.57 (s, 4H, CH₂CN), 3.42 (s, 4H, OCH₂C), 3.44 (t, J=6.5 Hz, 4H, octyl OCH₂); ¹³C NMR δ 116.7 (CN), 71.9 (CH₂CH₂OC), 70.8 (CCH₂OCH₂C), 41.3 (q C), 32.9 (CH₂CH₂CH₃), 29.45, 29.34 (2 octyl CH₂), 29.47 (OCH₂CH₂), 26.2 (CH₂CH₂CH₂O), 22.7 (CH₂CH₃), 21.7 (CH₂CN) 14.2 (Me); HR ESI MS m/z calcd for C₂₃H₄₂N₂O₂Na (M+Na) 401.3138. found 401.3152.

### Example 13B

### 3,3-Bis(decyloxymethyl)pentanedinitrile (13b)

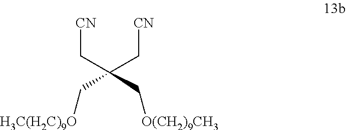

Treatment of compound 8b (Example 8B) (5.9 g, 9.3 mmol) in dry DMF (30 mL) with potassium cyanide (1.8 g, 28.0 mmol, 3.0 eq) following the procedure of Example 13A gives the title compound as a light yellow oil: yield 3.6 g, 91%; R_{F} 0.46 (hexanes:ethyl acetate 9:1); ¹H NMR δ 0.88 (t, 6H, J=6.9 Hz, 2×Me), 1.26-1.31 (br s, 28H, 14×CH₂), 1.53 (pentet, 4H, J=7.2 Hz, 2 OCH₂CH₂), 2.58 (s, 4H, CH₂N₃), 3.43 (s, 4H, OCH₂C), 3.45 (t, 4H, J=6.5 Hz, decyl OCH₂); ¹³C NMR δ 116.8 (CN), 72.0 (CH₂CH₂OC), 70.9 (OCH₂C), 41.3 (q C), 32.1 (CH₂CH₂CH₃), 29.75, 29.72, 29.53, 29.47 (4 decyl CH₂), 29.55 (OCH₂CH₂), 26.2 (CH₂CH₂CH₂O), 22.8 (CH₂CH₃), 21.7 (CH₂CN) 14.3 (Me); HR ESI MS m/z calcd for C₂₇H₅₀N₂O₂Na (M+Na) 457.3764. found 457.3781.

### Example 13C

### 3,3-Bis(dodecyloxymethyl)pentanedinitrile (13c)

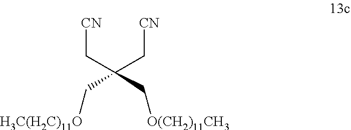

Treatment of compound 8c (Example 8C) (2.3 g, 3.3 mmol) in dry DMF (30 mL) with potassium cyanide (0.5 g, 8.4 mmol, 3.0 eq) following the procedure of Example 13A gives the title compound as a light yellow oil: yield 1.5 g, 91%; R_{F} 0.49 (hexanes:ethyl acetate 9:1); ¹H NMR δ 0.88 (t, 6H, J=6.9 Hz, 2×Me), 1.26-1.32 (br s, 36H, 18×CH₂), 1.54-1.58 (pentet, 4H, J=7.0 Hz, 2 OCH₂CH₂), 2.57 (s, 4H, CH₂CN), 3.38 (s, 4H, OCH₂C), 3.45 (t, 4H, J=7.0 Hz, 2 OCH₂CH₂), 2.57 (s, 4H, CH₂CN), 3.38 (s, 4H, OCH₂C), 3.45 (t, 4H, J=6.5 Hz, dodecyl OCH₂); ¹³C NMR δ 116.7 (CN), 72.0 (CH₂CH₂OC), 70.8 (CCH₂OCH₂C), 41.3 (q C), 32.0 (CH₂CH₂CH₃), 2×29.72, 2×29.70, 29.51, 29.45 (6 dodecyl CH₂), 29.53 (OCH₂CH₂), 26.2 (CH₂CH₂CH₂O), 22.8 (CH₂CH₃), 21.7 (CH₂CN) 14.3 (Me); HR ESI MS m/z calcd for C₃₁H₅₈N₂O₂Na (M+Na) 513.4391. found 513.4373.

### Example 13D

### 3,3-Bis(tetradecyloxymethyl)pentanedinitrile (13d)

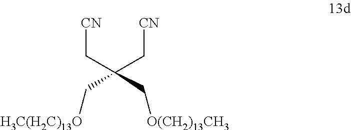

Treatment of compound 8d (Example 8D) (5.0 g, 6.7 mmol) in dry DMF (30 mL) with potassium cyanide (1.0 g, 16.7 mmol, 2.5 eq) following the procedure of Example 13A gives a light yellow oil that is taken up in hot 95% ethanol (50 mL). When this solution is kept at 5° C., the title compound (13d) precipitates as an amorphous solid, yield 2.24 g. Flash column chromatography of the residue yields an additional 0.57 g, total yield 2.81 g, 77%: R_{F} 0.56 (hexanes:ethyl acetate 9:1); mp 33-35° C.; ¹H NMR δ 0.88 (t, 6H, J=6.8 Hz, 2×Me), 1.22-1.36 (br s, 44H, 22×CH₂), 1.56 (pentet, 4H, J=6.7 Hz, 2OCH₂CH₂), 2.58 (s, 4H, CH₂CN), 3.43 (s, 4H, OCH₂C), 3.45 (t, J=6.5 Hz, 4H, tetradecyl OCH₂); ¹³C NMR δ 116.7 (CN), 72.0 (CH₂CH₂OC), 70.8 (CCH₂OCH₂C), 41.3 (q C), 32.1 (CH₂CH₂CH₃), 29.84, 29.78, 29.76, 29.72, 29.54, 29.51 (8 tetradecyl CH₂), 29.56 (OCH₂CH₂), 26.2 (CH₂CH₂CH₂O), 22.8 (CH₂CH₃), 21.8 (CH₂CN) 14.3 (Me); HR ESI MS m/z calcd for C₃₅H₆₆N₂O₂Na (M+Na) 569.5017. found 569.5002.

### Example 14A

### 3,3-bis(octyloxymethyl)pentanedioic acid (14a)

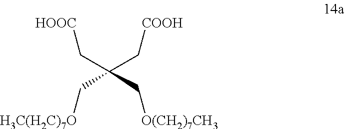

A mixture of compound 13a (Example 13A) (2.5 g, 5.61 mmol) in 1-propanol (40 mL) containing 35% NaOH (10 mL) is refluxed for 36 h. The reaction mixture is concentrated then the resulting aqueous reaction mixture is refluxed for another 24 h. The reaction mixture is cooled to 10° C. and acidified by adding a dilute HCl solution until the pH is 5 (pH paper). The mixture is extracted with ethyl acetate (2×50 mL) and the combined organic layers are washed with water (2×30 mL), brine (20 mL), dried (Na₂SO₄), and concentrated to give the crude product (2.5). The product is purified by flash column chromatography on silica gel using a gradient changing from 5% EtOAc in hexanes to 15% EtOAc in hexanes as eluent to give as a thick colorless syrup: yield 1.75 g (63%); R_{F} 0.5 (EtOAc:hexanes 1:1); ¹H NMR (CDCl₃) δ 0.88 (t, 6H, J=6.5 Hz, CH₃), 1.24-1.31 (m, 20H, 10×CH₂), 1.53 (pentet, 4H, J=6.5 Hz, OCH₂CH₂), 2.61 (s, 4H, COCH₂), 3.40 (t, 4H, J=6.5, OCH₂), 3.46 (s, 4H, OCH₂), 10.92 (bs, 2H, COOH); ¹³C NMR (CDCl₃) δ 176.8 (COOH), 73.2 (CCH₂O), 71.8 (OCH₂CH₂), 41.1 (q C), 37.5 (HOOCCH₂), 32.0 (CH₂CH₂CH₃), 29.60, 29.55, 29.42 (3 octyl CH₂), 26.3 (OCH₂CH₂CH₂), 22.8 (CH₃CH₂), 14.2 (CH₃); HR ESI MS m/z calcd for C₂₃H₄₃O₆ (M−H) 415.3065. found 415.3042.

### Example 14B

### 3,3-Bis(decyloxymethyl)pentanedioic acid (14b)

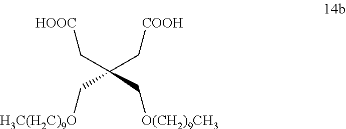

Reaction of compound 13b (Example 13B) (2.5 g, 5.8 mmol) in 1-propanol (40 mL) containing 35% NaOH (10 mL) following the procedure of Example 14A gives the title compound as a colorless solid: yield 1.2 g (44%); mp 68-71° C.′ R_{F} 0.4 (EtOAc:hexanes 1:1); ¹H NMR (CDCl₃) δ 0.87 (t, 6H, J=6.5 Hz, CH₃), 1.23-1.32 (m, 28H, 14×CH₂), 1.53 (pentet, 4H, J=6.5 Hz, OCH₂CH₂), 2.60 (s, 4H, COCH₂), 3.41 (t, 4H, J=6.5, OCH₂), 3.47 (s, 4H, OCH₂), 10.50 (bs, 2H, COOH); ¹³C NMR CDCl₃ δ 176.4 (COOH), 73.2 (CCH₂O), 71.8 (OCH₂CH₂), 41.1 (q C), 37.6 (HOOCCH₂), 32.1 (CH₂CH₂CH₃), 3×29.75, 29.60, 29.49 (5 decyl CH₂), 26.3 (OCH₂CH₂CH₂), 22.8 (CH₃CH₂), 14.3 (CH₃); HR ESI MS m/z calcd for C₂₇H₅₂O₆ (M−H) 471.3691. found 471.3667.

### Example 14C

### 3,3-Bis(dodecyloxymethyl)pentanedioic acid (14c)

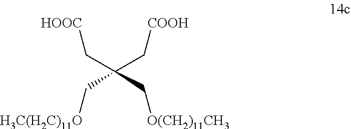

A mixture of compound 13c (Example 13C) (2.5 g, 5.8 mmol) in 1-propanol (40 mL) containing 35% NaOH (10 mL) is treated following the procedure of Example 14A except that the aqueous mixture is refluxed for 36 h to give the product as a colorless solid: yield 2.2 g (46%); mp 80-82° C.; R_{F} 0.46 (ethyl acetate:hexanes 1:1); ¹H NMR (CDCl₃) δ 0.88 (t, 6H, J=6.5 Hz, CH₃), 1.24-1.32 (m, 36H, 18×CH₂), 1.53 (pentet, 4H, J=6.5 Hz, OCH₂CH₂), 2.60 (s, 4H, COCH₂), 3.40 (t, 4H, J=6.5 Hz, OCH₂), 3.46 (s, 4H, OCH₂), 10.6 (bs, 2H, COOH); ¹³C NMR (CDCl₃) δ 176.5 (COOH), 73.2 (CCH₂O), 71.8 (OCH₂CH₂), 41.1 (qC), 37.6 (HOOCCH₂), 32.1 (CH₂CH₂CH₃), 3×29.84, 2×29.81, 29.61, 29.52 (7 dodecyl CH₂), 26.3 (OCH₂CH₂CH₂), 22.8 (CH₃CH₂), 14.3 (CH₃); HR ESI MS m/z calcd for C₃₁H₅₉O₆ (M−H) 527.4317. found 527.4324.

### Example 14D

### 3,3-Bis(tetradecyloxymethyl)pentanedioic acid (14d)

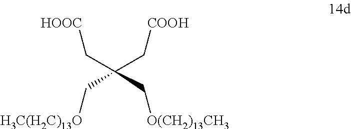

A mixture of compound 13d (Example 13D) (5.0 g, 6.7 mmol) in 1-propanol (40 mL) containing 35% NaOH (10 mL) is treated following the procedure of Example 14C to give the product as a colorless solid: yield: 2.2 g (46%); mp 84-86° C.; R_{F} 0.51 (ethyl acetate:hexanes 1:1); ¹H NMR (CDCl₃) δ 0.88 (t, 6H, J=6.5 Hz, CH₃), 1.22-1.32 (m, 44 H, 22×CH₂), 1.54 (pentet, 4H, J=6.5 Hz, OCH₂CH₂), 2.60 (s, 4H, COCH₂), 3.41 (t, 4H, J=6.5 Hz, OCH₂), 3.47 (s, 4H, OCH₂), 10.6 (bs, 2H, COOH); ¹³C NMR (CDCl₃) δ 175.6 (COOH), 73.3 (CCH₂O), 71.9 (OCH₂CH₂), 41.1 (qC), 37.8 (HOOCCH₂), 32.1 (CH₂CH₂CH₃), 29.86, 29.82, 29.60, 29.52 (tetradecyl CH₂), 26.3 (OCH₂CH₂CH₂), 22.8 (CH₃CH₂), 14.3 (CH₃); HR ESI MS m/z calcd for C₃₅H₆₈O₆ (M−H) 583.4943. found 583.4978.

### Example 15A

### N,N,N′,N′-tetramethyl-3,3-bis(octyloxymethyl)pentanediamide (15a)

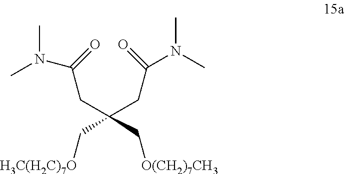

1-Hydroxybenzotriazole (HOBT, 1.03 g, 7.69 mmol) and N-(3-dimethylaminopropyl)-N′-ethylcarbodiimide hydrochloride (EDC.HCl, 1.54 g, 8.07 mmol) are added to a stirred solution of diacid 14a (Example 14A) (1.6 g, 3.9 mmol) and the reaction mixture is stirred for 1 h. Dimethylamine hydrochloride (1.25 g, 15.4 mmol) and triethylamine (2.7 g, 27 mmol) are added and the reaction mixture is stirred for another 24 h, then diluted with dichloromethane (50 mL). This mixture is washed with water (2×30 mL), brine (20 mL), dried (Na₂SO₄) and concentrated. The residue is purified by flash column chromatography using a gradient of 15 to 30% EtOAc in hexanes as eluent, giving compound 15a as a viscous liquid: yield 1.63 g (91%); R_{F} 0.5 (ethyl acetate:hexanes 2:1); ¹H NMR (CDCl₃) δ 0.88 (t, 6H, J=6.5 Hz, CH₃), 1.26-1.30 (m, 20H, 10×CH₂), 1.51 (pentet, 4H, J=6.5 Hz, OCH₂CH₂), 2.65 (s, 4H, COCH₂), 2.89 (s, 6H, NCH₃), 3.03 (s, 6H, NCH₃), 3.36 (t, 4H, J=6.5 Hz, OCH₂), 3.53 (s, 4H, OCH₂); ¹³C NMR (CDCl₃) δ 172.4 (CO), 72.7 (CCH₂O), 71.4 (OCH₂CH₂), 42.1 (qC), 37.9 (NCH₃), 35.5 (NCH₃), 33.6 (NCOCH₂), 32.0 (CH₂CH₂CH₂), 29.87, 29.63, 29.51 (2×3 octyl CH₂), 26.4 (OCH₂CH₂CH₂), 22.8 (CH₃CH₂), 14.2 (CH₃); HR ESI MS m/z calcd for C₂₇H₅₄N₂O₄Na (M+Na) 493.3976. found 493.3966.

### Example 15B

### 3,3-Bis(decyloxymethyl)-N,N,N′,N′-tetramethylpentanediamide (15b)

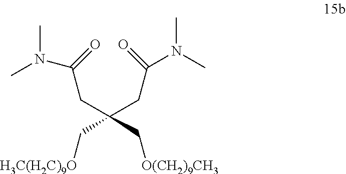

1-Hydroxybenzotriazole (HOBT, 1.14 g, 8.45 mmol), EDC.HCl (1.61 g, 8.45 mmol), diacid 14b (Example 14B) (1.9 g, 4.0 mmol), dimethylamine hydrochloride (1.31 g, 16.1 mmol) and triethylamine (3.26 g, 32.2 mmol) are reacted following the procedure of Example 15A, giving compound 15b as a viscous liquid: yield 2.0 g (95%); R_{F} 0.5 (ethyl acetate:hexanes 2:1); ¹H NMR (CDCl₃) δ 0.89 (t, 6H, J=6.5 Hz, CH₃), 1.26-1.35 (m, 28H, 14×CH₂), 1.53 (pentet, 4H, J=6.5 Hz, OCH₂CH₂), 2.67 (s, 4H, COCH₂), 2.91 (s, 6H, NCH₃), 3.04 (s, 6H, NCH₃), 3.38 (t, 4H, J=6.5 Hz, OCH₂), 3.54 (s, 4H, OCH₂); ¹³C NMR (CDCl₃) δ 172.4 (CO), 72.7 (CCH₂O), 71.4 (OCH₂CH₂), 42.1 (qC), 38.0 (NCH₃), 35.5 (NCH₃), 33.6 (NCOCH₂), 32.1 (CH₃CH₂CH₂), 2×29.87, 29.78, 29.69, 29.52 (2×4 decyl CH₂), 26.4 (OCH₂CH₂CH₂), 22.8 (CH₃CH₂), 14.3 (CH₃); HR ESI MS m/z calcd for C₃₁H₆₂N₂O₄Na (M+Na) 549.4602. found 549.4580.

### Example 15C

### 3,3-Bis(dodecyloxymethyl)-N,N,N′,N′-tetramethylpentanediamide (15c)

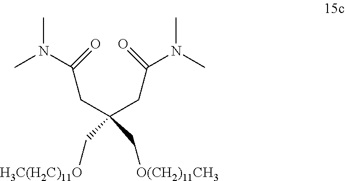

1-Hydroxybenzotriazole (HOBT, 0.96 g, 7.2 mmol), EDC.HCl (1.36 g, 7.15 mmol), diacid 14b (Example 14C) (1.8 g, 3.4 mmol), dimethylamine hydrochloride (1.11 g, 13.6 mmol) and triethylamine (2.06 g, 20.5 mmol) are reacted following the procedure of Example 15A to give the title compound (15c) as a viscous liquid: yield 1.7 g (86%); R_{F} 0.5 (ethyl acetate:hexanes 2:1); ¹H NMR (CDCl₃) δ 0.89 (t, 6H, J=6.5 Hz, CH₃), 1.22-1.34 (m, 36H, 18×CH₂), 1.50 (pentet, 4H, J=6.5 Hz, OCH₂CH₂), 2.65 (s, 4H, COCH₂), 2.89 (s, 6H, NCH₃), 3.03 (s, 6H, NCH₃), 3.36 (t, 4H, J=6.5 Hz, OCH₂), 3.52 (s, 4H, OCH₂); ¹³C NMR (CDCl₃) δ 172.4 (CO), 72.7 (CCH₂O), 71.4 (OCH₂CH₂), 42.1 (qC), 37.9 (NCH₃), 35.5 (NCH₃), 33.5 (NCOCH₂), 32.1 (CH₃CH₂CH₂), 29.88 (×3), 29.83 (×2), 29.70, 29.52 (7 dodecyl CH₂), 26.4 (OCH₂CH₂CH₂), 22.8 (CH₃CH₂), 14.2 (CH₃); HR ESI MS m/z calcd for C₃₅H₇₀N₂O₄Na (M+Na) 605.5228. found 605.5183.

### Example 15D

### N,N,N′,N′-Tetramethyl-3,3-bis(tetradecyloxymethyl)pentanediamide (15d)

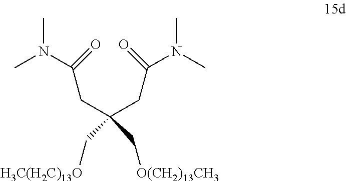

1-Hydroxybenzotriazole (HOBT, 2.0 g, 15 mmol), EDC.HCl (2.89 g, 15.1 mmol), diacid 14d (Example 14D) (4.2 g, 7.2 mmol), dimethylamine hydrochloride (2.40 g, 29.5 mmol) and triethylamine (5.81 g, 57.5 mmol) are reacted following the procedure of Example 15A to give the title compound (15d) as a solid: yield 4.1 g (89%); mp 52-54° C.; R_{F} 0.46 (ethyl acetate:hexanes 2:1); ¹H NMR (CDCl₃) δ 0.88 (t, 6H, J=7.0 Hz, CH₃), 1.26-1.42 (m, 44H, 22×CH₂), 1.51 (p, 4H, J=6.5 Hz, OCH₂CH₂), 2.65 (s, 4H, COCH₂), 2.89 (s, 6H, NCH₃), 3.02 (s, 6H, NCH₃), 3.36 (t, 4H, J=6.5 Hz, OCH₂), 3.52 (s, 4H, OCH₂); ¹³C NMR (CDCl₃) δ 172.4 (CO), 72.7 (CCH₂O), 71.4 (OCH₂CH₂), 42.1 (qC), 37.9 (NCH₃), 35.5 (NCH₃), 33.6 (NCOCH₂), 32.1 (CH₃CH₂CH₂), 29.9-29.8 (7C), 29.69, 29.52 (2×9 tetradecyl CH₂), 26.4 (OCH₂CH₂CH₂), 22.9 (CH₃CH₂), 14.3 (CH₃); HR ESI MS m/z calcd for C₃₉H₇₈H₂O₄Na (M+Na) 661.5854. found 661.5823.

### Example 16A

### N,N,N′,N′-Tetramethyl-3,3-bis(octyloxymethyl)-1,5-pentanediamine (16a)

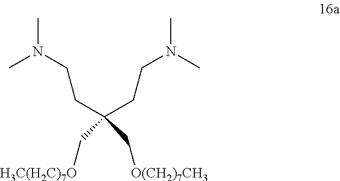

Diamide 15a (Example 15A) (0.9 g, 1.91 mmol) is added dropwise to a stirred suspension of LiAlH₄ (0.29 g, 7.65 mmol) in THF (50 mL) at 0° C., then the reaction mixture is stirred at rt for 6 h. Ethyl acetate (50 mL) is added dropwise, followed by water (0.3 mL), then 1M NaOH (0.3 mL). The mixture is filtered on a bed of Celite™ that is washed with hot ethyl acetate. The combined filtrate and washings are dried (Na₂SO₄), filtered and concentrated to a residue that is purified by flash column chromatography. Elution using a gradient of 5 to 15% MeOH in dichloromethane gives compound 16a as a light brown liquid: yield: 0.67 g (80%); R_{F} on basic alumina 0.46 (dichloromethane: methanol 96:4); ¹H NMR (CDCl₃) δ 0.88 (t, 6H, J=7.0 Hz, CH₃), 1.22-1.38 (m, 20H, 10×CH₂), 1.43-1.54 (m, 8H, NCH₂CH₂, OCH₂CH₂), 2.21 (s, 12H, NCH₃), 2.27 (t, 4H, J=8.0 Hz, NCH₂), 3.19 (s, 4H, OCH₂), 3.34 (t, 4H, J=6.5 Hz, OCH₂); ¹³C NMR (CDCl₃) δ 73.8 (CCH₂O), 71.5 (OCH₂CH₂), 54.5 (NCH₂), 45.8 (NCH₃), 40.2 (qC), 32.0 (CH₃CH₂CH₂), 30.3 (NCH₂CH₂), 29.89, 29.64, 29.50 (2×3 octyl CH₂), 26.5 (OCH₂CH₂CH₂), 22.8 (CH₃CH₂), 14.3 (CH₃); HR ESI MS m/z calcd for C₂₇H₅₈N₂O₂ (M+1) 443.4571. found 443.4558.

### Example 16B

### 3,3-Bis(decyloxymethyl)-N,N,N′,N′-tetramethyl-1,5-pentanediamine (16b)

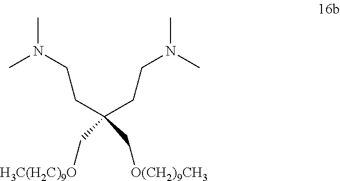

Reaction of diamide 15b (Example 15B) (1.9 g, 3.6 mmol) with LiAlH₄ (0.54 g, 14 mmol) in THF (50 mL) following the procedure of Example 16A gives compound 16b as light brown liquid: yield: 1.1 g (61%); R_{F} on basic alumina 0.44 (dichloromethane: methanol 96:4); ¹H NMR (CDCl₃) δ 0.88 (t, 6H, J=7.0 Hz, CH₃), 1.22-1.45 (m, 28H, 14×CH₂), 1.49-1.54 (m, 8H, NCH₂CH₂, OCH₂CH₂), 2.27 (s, 12H, NCH₃), 2.37 (t, 4H, J=7.5, NCH₂), 3.19 (s, 4H, OCH₂), 3.34 (t, 4H, J=6.5 Hz, OCH₂); ¹³C NMR (CDCl₃) δ 73.8 (CCH₂O), 71.6 (OCH₂CH₂), 54.5 (NCH₂), 45.5 (NCH₃), 40.3 (qC), 32.1 (CH₃CH₂CH₂), 30.01 (NCH₂CH₂), 29.87, 29.85, 29.79, 29.68, 29.52 (2×5 decyl CH₂), 26.5 (OCH₂CH₂CH₂), 22.9 (CH₃CH₂), 14.3 (CH₃); HR ESI MS m/z calcd for C₂₇H₅₈N₂O₂ (M+1) 443.4571. found 443.4558.

### Example 16C

### 3,3-Bis(dodecyloxymethyl)-N,N,N′,N′-tetramethyl-1,5-pentanediamine (16c)

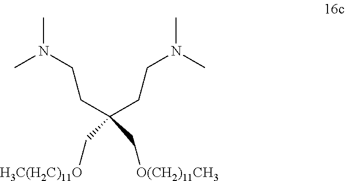

Reaction of diamide 15c (Example 15C) (1.9 g, 3.3 mmol) with LiAlH₄ (0.62 g, 16 mmol) in THF (50 mL) following the procedure of Example 16A gives compound 16c as a light brown liquid: yield 1.3 g (72%); R_{F} on basic alumina 0.52 (DCM: methanol 95:5); ¹H NMR (CDCl₃) δ 0.88 (t, 6H, J=7.0 Hz, CH₃), 1.22-1.45 (m, 36H, 18×CH₂), 1.45-1.54 (m, 8H, NCH₂CH₂, OCH₂CH₂), 2.21 (s, 12H, NCH₃), 2.26-2.29 (m, 4H, J=8.5 Hz, NCH₂), 3.18 (s, 4H, OCH₂), 3.34 (t, 4H, J=6.5 Hz, OCH₂); ¹³C NMR (CDCl₃) δ 73.8 (CCH₂O), 71.5 (OCH₂CH₂), 54.5 (NCH₂), 45.8 (NCH₃), 40.2 (qC), 32.1 (CH₂CH₂CH₂), 30.2 (NCH₂CH₂), 29.9-20.8 (4C), 29.69, 29.52 (2×7 dodecyl CH₂), 26.5 (OCH₂CH₂CH₂), 22.8 (CH₃CH₂), 14.3 (CH₃); HR ESI MS m/z calcd for C₃₅H₇₄N₂O₂(M+1) 555.5823. found 555.5854.

### Example 16D

### N,N,N′,N′-Tetramethyl-3,3-bis(tetradecyloxymethyl)-1,5-pentanediamine (16d)

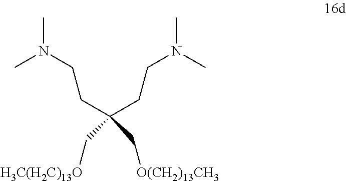

Reaction of diamide 15d (Example 15D) (2.2 g, 3.4 mmol) with LiAlH₄ (0.65 g, 17 mmol) in THF (50 mL) following the procedure of Example 16A gives a residue that is purified by flash column chromatography. Elution using a gradient of 10 to 15% MeOH in DCM gives compound 16d as a light brown liquid: yield 1.7 g (81%); R_{F} on basic alumina 0.55 (DCM:methanol 95:5); ¹H NMR (CDCl₃) δ 0.88 (t, 6H, J=7.0 Hz, CH₃), 1.21-1.37 (m, 44H, 22×CH₂), 1.46-1.54 (m, 8H, NCH₂CH₂, OCH₂CH₂), 2.23 (s, 12H, NCH₃), 2.30-2.33 (m, 4H, J=8.0 Hz, NCH₂), 3.18 (s, 4H, OCH₂), 3.34 (t, 4H, J=6.5 Hz, OCH₂); ¹³C NMR (CDCl₃) δ 73.7 (CCH₂O), 71.5 (OCH₂CH₂), 54.4 (NCH₂), 45.5 (NCH₃), 40.2 (qC), 32.1 (CH₂CH₂CH₃), 30.0 (NCH₂CH₂), 29.9-29.8, 29.68, 29.51 (tetradecyl CH₂), 26.9 (OCH₂CH₂CH₂), 22.8 (CH₃CH₂), 14.3 (CH₃); HR ESI MS m/z calcd for C₃₉H₈₂N₂O₂ (M+1) 611.6449. found 611.6466.

### Example 17A

### N,N,N,N′,N′,N′-hexamethyl-3,3-bis(octyloxymethyl)-1,5-pentanediammonium diiodide (17a)

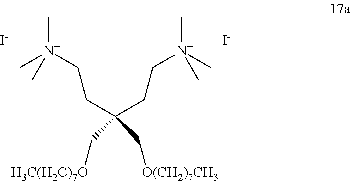

A solution of methyl iodide (1.6 g, 11 mmol) and diamine 16a (Example 16A) (0.50 g, 1.1 mmol) in THF (20 mL) is refluxed for 36 h, then concentrated. The solid residue is purified by flash column chromatography using 10% methanol in dichloromethane as eluent to give the title product as an off-white solid: yield 0.70 g (85%); mp 223-225° C.; R_{F} 0.5 on basic alumina (8% methanol in dichloromethane); ¹H NMR (CDCl₃) δ 0.88 (t, 6H, J=7.0 Hz, CH₃), 1.22-1.28 (m, 20H, 10×CH₂), 1.45 (t, 4H, J=6.0 Hz, OCH₂CH₂), 1.83 (AA′ part of AA′XX′ pattern, 4H, NCH₂CH₂), 3.13 (s, 4H, OCH₂), 3.15 (s, 18H, NCH₃), 3.18 (t, 4H, J=7.0 Hz, OCH₂), 3.73 (XX part of AA′XX′ pattern, 4H, NCH₂); ¹³C NMR (CDCl₃) δ 71.7 (CCH₂O), 71.5 (OCH₂CH₂), 62.8 (CH₂N(CH₃)₃), 54.2 (NCH₃), 41.6 (qC), 32.0 (CH₂CH₂CH₂), 29.82, 29.58, 29.50 (3 octyl CH₂), 26.6 (OCH₂CH₂CH₂), 24.6 (NCH₂CH₂), 22.8 (CH₃CH₂), 14.3 (CH₃); HR ESI MS m/z calcd for C₂₉H₆₄IN₂O₂ (M−I) 599.4007. found 599.3993.

### Example 17B

### 3,3-Bis(decyloxymethyl)-N,N,N,N′,N′,N′-hexamethyl-1,5-pentanediammonium diiodide (17b)

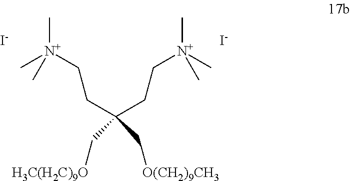

Reaction of methyl iodide (1.93 g, 13.7 mmol) and diamine 16b (Example 16B) (0.68 g, 1.4 mmol) in THF following the procedure of Example 17A gives the title product as an off-white solid: yield 0.80 g (80%); mp 214-216° C.; R_{F} 0.5 on basic alumina (7% methanol in dichloromethane); ¹H NMR (CDCl₃) δ 0.88 (t, 6H, J=7.0 Hz, CH₃), 1.24-1.35 (m, 28H, 14×CH₂), 1.52 (t, 4H, J=6.5 Hz, OCH₂CH₂), 1.93 (AA′ part of AA′XX′ pattern, 4H, NCH₂CH₂), 3.39 (s, 4H, OCH₂), 3.42 (s, 18H, NCH₃), 3.44 (t, 4H, J=7.0 Hz, OCH₂), 4.02 (XX part of AA′XX′ pattern, 4H, NCH₂); ¹³C NMR (CDCl₃) δ 71.7 (CCH₂O), 71.5 (OCH₂CH₂), 62.8 (CH₂N(CH₃)₃), 54.2 (NCH₃), 41.5 (qC), 32.1 (CH₂CH₂CH₂), 29.85, 29.81, 29.77, 29.62, 29.47 (5 decyl CH₂), 26.5 (OCH₂CH₂CH₂), 24.6 (NCH₂CH₂), 22.9 (CH₃CH₂), 14.4 (CH₃); HR ESI MS m/z calcd for C₃₃H₇₂IN₂O₂ (M−I) 655.4633. found 655.4614.

### Example 17C

### 3,3-Bis(dodecyloxymethyl)-N,N,N,N′,N′,N′-hexamethyl-1,5-pentanediammonium diiodide (17c)

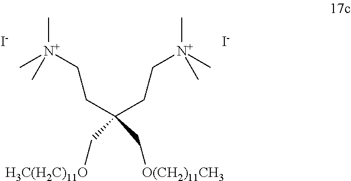

Reaction of methyl iodide (2.56 g, 18.0 mmol) with diamine 16c (Example 16C) (1.00 g, 1.80 mmol) following the procedure of Example 17A gives the title product as an off-white solid: yield 1.20 g (78%); mp 220-223° C.; R_{F} 0.5 on basic alumina (7% methanol in dichloromethane); ¹H NMR (CDCl₃) δ 0.88 (t, 6H, J=7.0 Hz, CH₃), 1.23-1.36 (m, 36 H, 18×CH₂), 1.52 (t, 4H, J=6.5 Hz, OCH₂CH₂), 1.89 (AA′ part of AA′XX′ pattern, 4H, NCH₂CH₂), 3.38 (s, 4H, OCH₂), 3.44 (t, 4H, J=7.0 Hz, OCH₂), 3.45 (s, 18H, NCH₃), 3.94 (XX′ part of AA′XX′ pattern, 4H, NCH₂); ¹³C NMR (CDCl₃) 71.7 (CCH₂O), 71.5 (OCH₂CH₂), 62.8 (CH₂N(CH₃)₃), 54.2 (NCH₃), 41.5 (qC), 32.1 (CH₂CH₂CH₂), 29.90-29.75 (5C), 29.62, 29.47 (7 dodecyl CH₂), 26.5 (OCH₂CH₂CH₂), 24.6 (NCH₂CH₂), 22.9 (CH₃CH₂), 14.3 (CH₃); HR ESI MS m/z calcd for C₃₇H₈₀IN₂O₂ (M−I) 711.5259. found 711.5239.

### Example 17D

### N,N,N,N′,N′,N′-Hexamethyl-3,3-bis(tetradecyloxymethyl)-1,5-pentanediammonium diiodide (17d)

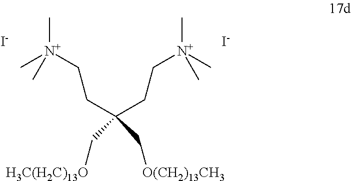

Reaction of methyl iodide (2.32 g, 16.4 mmol) and diamine 16d (Example 16D) (1.0 g, 1.6 mmol) following the procedure of Example 17A (without chromatography) gives a solid residue that is crystallized from dichloromethane and dried under vacuum to give compound 17d as an off-white shiny solid: yield 1.2 g (82%); mp 218-221° C.; R_{F} 0.6 on basic alumina (7% methanol in dichloromethane); ¹H NMR (CDCl₃) δ 0.88 (t, 6H, J=7.0 Hz, CH₃), 1.27 (m, 44H, 22×CH₂), 1.52 (t, 4H, J=6.5 Hz, OCH₂CH₂), 1.93 (AA′ part of AA′XX′ pattern, 4H, NCH₂CH₂), 3.39 (s, 4H, OCH₂), 3.43 (s, 18H, NCH₃), 3.44 (t, 4H, J=7.0 Hz, OCH₂), 4.03 (XX′ part of AA′XX′ pattern, 4H, NCH₂); ¹³C NMR (CDCl₃) δ 71.7 (CCH₂O), 71.5 (OCH₂), 62.8 (CH₂N(CH₃)₃), 54.1 (NCH₃), 41.4 (qC), 32.1 (CH₂CH₂CH₂), 29.90-29.75 (7C), 29.64, 29.50 (tetradecyl CH₂), 26.5 (OCH₂CH₂CH₂), 24.6 (NCH₂CH₂), 22.8 (CH₃CH₂), 14.2 (CH₃); HR ESI MS m/z calcd for C₄₁H₈₈IN₂O₂ (M−1) 767.5885. found 767.5907.

### Example 18A

### N,N,N′,N′-tetramethyl-4,4-bis(octyloxymethyl)-2,5-heptadienediamide (18a)

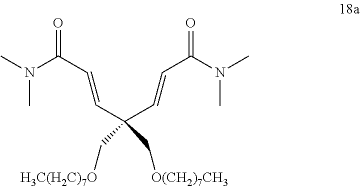

A solution of dry dimethyl sulfoxide (DMSO) (0.47 g, 6.11 mmol) in dichloromethane (2 mL) is added dropwise to a stirred solution of oxalyl chloride (0.38 g, 3.0 mmol) in dichloromethane (5 mL) at −78° C. After the reaction mixture had been stirred for 30 min, a solution of diol 2a (Example 2A) (0.50 g, 1.4 mmol) is added and the reaction mixture is stirred for 1.5 h at −78° C., then Et₃N (0.98 g, 9.7 mmol) is added slowly. The reaction mixture is stirred for 30 min, allowed to warm to rt, the quenched by addition of a saturated NH₄Cl solution (10 mL). The reaction mixture is extracted with dichloromethane (3×50 mL), and the combined organic layers are washed with 2M HCl (5 mL), water (2×5 mL), brine (5 mL), then dried (Na₂SO₄) and concentrated to give 2,2-bis(octyloxymethyl)propanedial as a colorless viscous oil: yield 0.47 g, 95%; R_{F} 0.56 (hexanes:ethyl acetate 8:2); ¹H NMR δ 0.88 (t, 6H, J=7.0 Hz, 2×Me), 1.26-1.32 (br s, 20H, 10×CH₂), 1.51 (p, 4H, J=7.2 Hz, 2 OCH₂CH₂), 3.42 (t, 4H, J=6.5 Hz, decyl OCH₂), 3.87 (s, 4H, OCH₂C), 9.74 (s, 2H, CHO); ¹³C NMR δ 199.4 (CHO), 72.3 (CH₂CH₂OC), 68.6 (OCH₂C), 43.8 (q C), 31.7 (CH₂CH₂CH₃), 29.76, 29.62, 29.48, 29.44, 29.37 (5 decyl CH₂), 26.12 (CH₂CH₂CH₂O), 22.8 (CH₂CH₃), 14.2 (Me); LR ESI m/z calcd for C₂₁H₄₀O₄Na.MeOH 411.31. found 411.3; for C₂₁H₄₀O₄Na.2MeOH 443.33. found 443.3; for 2C₂₁H₄₀O₄+Na+H₂O 753.54. found 753.6.

Diethyl N,N-dimethylcarbamoylmethylphosphonate (prepared by the method of Paul A. Bartlett, Nicholas I. Carruthers, Beat M. Winter, and Karen P. Long. J. Org. Chem. 47, 1284-1291, 1982) (4.1 g, 18 mmol) in THF (10 mL) is added in portions to a stirred suspension of NaH (0.45 g, 18 mmol) in THF (80 mL) at rt and the reaction mixture is stirred for 2 h, then cooled to 0° C. A solution of 2,2-bis(octyloxymethyl)propanedial (1.6 g, 4.5 mmol) in THF (10 mL) is added and the reaction mixture is stirred for 24 h at rt. A saturated aqueous ammonium chloride solution (20 mL) is added to the reaction mixture and then the volatile organic components are removed by concentration. The resulting solution is extracted with ethyl acetate (3×30 mL). The combined organic layers are washed with water (2×20 mL), brine (20 mL), dried (Na₂SO₄), filtered and concentrated. The residue is purified by flash column chromatography using a gradient of 80 to 90% EtOAc in hexanes as eluent, yielding compound 18a as a viscous liquid: yield 1.4 g (63%); R_{F} 0.35 (dichloromethane:methanol 94:6); ¹H NMR δ 0.88 (t, 6H, J=7 Hz, 2×Me), 1.26-1.34 (br s, 20H, 10×CH₂), 1.53 (pentet, 4H, J=7 Hz, 2 OCH₂CH₂), 2.99 (s, 3H, NCH₃), 3.05 (s, 3H, NCH₃), 3.39 (t, 4H, J=6.5 Hz, decyl OCH₂), 3.49 (s, 4H, OCH₂C), 6.36 (d, 2H, J=16 Hz, COCHCH), 6.86 (d, 2H, J=16 Hz, COCHCH); ¹³C NMR δ 166.8 (C═O), 145.3 (CH═CHC), 121.9 (COCH═), 72.9 (CH₂CH₂OC), 71.9 (OCH₂C), 48.9 (q C), 37.5 (NCH₃), 35.8 (NCH₃), 32.0 (CH₂CH₂CH₃), 29.77, 29.61, 29.45 (3 octyl CH₂), 26.4 (CH₂CH₂CH₂O), 22.8 (CH₂CH₃), 14.2 (Me); HR ESI MS m/z calcd for C₂₉H₅₄N₂O₄Na (M+Na) 517.3976. found 517.3971.

### Example 18B

### 4,4-Bis(decyloxymethyl)-N,N,N′,N′-tetramethyl-2,5-heptadienediamide (18b)

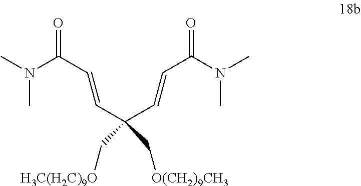

Treatment of diol 2b (Example 2B) (2.3 g, 5.5 mmol) in dry dichloromethane with the Swern oxidation mixture [DMSO (1.89 g, 24.3 mmol), oxalyl chloride (1.54 g, 12.2 mmol)] followed by workup, following the procedure of Example 18A, gives 2,2-bis(decyloxymethyl)propanedial as a light yellow oil: yield 2.1 g, 92%; R_{F} 0.7 (hexanes:ethyl acetate 8:2); ¹H NMR δ 0.88 (t, 6H, J=6.5 Hz, 2×Me), 1.25-1.31 (brs, 28H, 14×CH₂), 1.51 (pentet, 4H, J=7.2 Hz, 2 OCH₂CH₂), 3.41 (t, 4H, J=6.5 Hz, decyl OCH₂), 3.87 (s, 4H, OCH₂C), 9.74 (s, 2H, CHO); ¹³C NMR δ 199.5 (CHO), 72.3 (CH₂CH₂OC), 68.6 (OCH₂C), 43.7 (q C), 32.0 (CH₂CH₂CH₃), 29.72, 29.53, 29.46 (decyl CH₂), 26.13 (CH₂CH₂CH₂O), 22.8 (CH₂CH₃), 14.3 (Me); LR ESI MS m/z calcd for C₂₅H₄₈O₄Na.MeOH 467.37. found 467.4.

A mixture of diethyl N,N-dimethylcarbamoylmethylphosphonate (4.54 g, 20.4 mmol), NaH (0.49 g, 20.6 mmol) and 2,2-bis(decyloxymethyl)propanedial (2.0 g, 4.8 mmol) in THF (100 mL) is treated following the procedure of Example 18A to give compound 18b as a colorless liquid: yield, 1.7 g (64%); R_{F} 0.39 (dichloromethane: methanol 95:5); ¹H NMR δ 0.88 (t, 6H, J=7 Hz, 2×Me), 1.26-1.31 (br s, 28H, 14×CH₂), 1.52 (pentet, 4H, J=7 Hz, 2 OCH₂CH₂), 2.99 (s, 3H, NCH₃), 3.02 (s, 3H, NCH₃), 3.39 (t, 4H, J=6.5 Hz, decyl OCH₂), 3.49 (s, 4H, OCH₂C), 6.36 (d, 2H, J=15.5 Hz, COCH═), 6.86 (d, 2H, J=15.5 Hz, ═CHC); ¹³C NMR δ 166.8 (C═O), 145.3 (CH═CHC), 121.9 (COCH═), 72.9 (CH₂CH₂OC), 71.9 (OCH₂C), 48.9 (q C), 37.5 (NCH₃), 35.8 (NCH₃), 32.0 (CH₂CH₂CH₃), 29.79, 29.76, 29.66, 29.49 (5 decyl CH₂), 26.4 (CH₂CH₂CH₂O), 22.8 (CH₂CH₃), 14.2 (Me); HR ESI MS m/z calcd for C₃₃H₆₂N₂O₄Na (M+Na) 573.4602. found 573.4592.

### Example 18C

### 4,4-Bis(dodecyloxymethyl)-N,N,N′,N′-tetramethyl-2,5-heptadienediamide (18c)

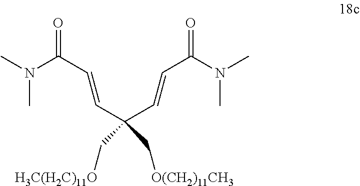

Treatment of diol 2c (Example 2C) (1.3 g, 2.8 mmol) in dry dichloromethane (30 mL) with the Swern oxidation mixture [DMSO (1.74 g, 22.3 mmol), oxalyl chloride (1.42 g, 11.2 mmol)] following the procedure of Example 18A gives 2,2-bis(dodecyloxymethyl)propanedial as a light yellow oil: yield 1.2 g, 92%; R_{F} 0.7 (hexanes:ethyl acetate 8:2); ¹H NMR δ 0.88 (t, 6H, J=7 Hz, 2×Me), 1.25-1.32 (br s, 36H, 18×CH₂), 1.52 (pentet, 4H, J=7.2 Hz, 2 OCH₂CH₂), 3.41 (t, 4H, J=6.5 Hz, decyl OCH₂), 3.87 (s, 4H, OCH₂C), 9.74 (s, 2H, CHO); ¹³C NMR δ 199.4 (CHO), 72.3 (CH₂CH₂OC), 68.6 (OCH₂C), 43.7 (q C), 32.1 (CH₂CH₂CH₃), 29.81, 29.78, 29.72, 29.53, 29.44, (decyl CH₂), 26.1 (CH₂CH₂CH₂O), 22.8 (CH₂CH₃), 14.2 (Me).

A mixture of diethyl N,N-dimethylcarbamoylmethylphosphonate (2.67 g, 11.9 mmol), NaH (0.29 g, 12 mmol) and 2,2-bis(dodecyloxymethyl)propanedial (1.1 g, 2.3 mmol) in THF (100 mL) is treated following the procedure of Example 18A to give compound 18c as a colorless solid: yield 1.25 g (69%); mp 52-54° C.; R_{F} 0.42 (dichloromethane:methanol 95:5); ¹H NMR δ 0.88 (t, 6H, J=7 Hz, 2×Me), 1.26-1.32 (br s, 36H, 18×CH₂), 1.52 (pentet, 4H, J=7 Hz, 2 OCH₂CH₂), 2.99 (s, 3H, NCH₃), 3.05 (s, 3H, NCH₃), 3.39 (t, 4H, J=6.5 Hz, decyl OCH₂), 3.49 (s, 4H, OCH₂C), 6.36 (d, 2H, J=15.5 Hz, COCH═), 6.86 (d, 2H, J=15.5 Hz, ═CHC); ¹³C NMR δ 166.8 (C═O), 145.3 (CH═CHC), 121.9 (COCH═), 72.9 (CH₂CH₂OC), 71.9 (OCH₂C), 48.9 (qC), 37.5 (NCH₃), 35.8 (NCH₃), 32.1 (CH₂CH₂CH₃), 29.86, 29.82, 29.68, 29.52 (5 dodecyl CH₂), 26.4 (CH₂CH₂CH₂O), 22.8 (CH₂CH₃), 14.3 (Me); HR ESI MS m/z calcd for C₃₇H₇₀N₂O₄Na (M+Na) 629.5228. found 629.5244.

### Example 18D

### N,N,N′,N′-Tetramethyl-4,4-bis(tetradecyloxymethyl)-2,5-heptadienediamide (18d)

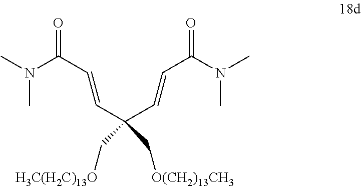

Treatment of diol 2d (Example 2D) (3.0 g, 5.7 mmol) in dry DCM (30 mL) with the Swern oxidation mixture [DMSO (2.03 g, 26.1 mmol), oxalyl chloride (1.65 g, 13.0 mmol)] following the procedure of Example 18A gives 2,2-bis(tetradecyloxymethyl)propanedial as a light yellow oil: yield 2.5 g, 86%; R_{F} 0.7 (hexanes:ethyl acetate 8:2); ¹H NMR δ 0. 0.88 (t, 6H, J=7 Hz, 2×Me), 1.25-1.32 (br s, 36H, 18×CH₂), 1.51 (pentet, 4H, J=7.2 Hz, 2 OCH₂CH₂), 3.40 (t, 4H, J=6.5 Hz, decyl OCH₂), 3.87 (s, 4H, OCH₂C), 9.74 (s, 2H, CHO); ¹³C NMR δ 199.5 (CHO), 72.4 (CH₂CH₂OC), 68.6 (OCH₂C), 43.7 (q C), 32.1 (CH₂CH₂CH₃), 29.81, 29.77, 29.52, 29.45 (decyl CH₂), 26.1 (CH₂CH₂CH₂O), 22.9 (CH₂CH₃), 14.2 (Me); LR ESI MS m/z calcd for C₃₃H₆₄O₄Na.MeOH, 579.50. found 579.5.

A mixture of diethyl N,N-dimethylcarbamoylmethylphosphonate (4.68 g, 20.9 mmol), NaH (0.51 g, 21 mmol) and 2,2-bis(tetradecyloxymethyl)propanedial (2.5 g, 4.7 mmol) in THF (100 mL) is treated following the procedure of Example 18A to give compound 18d as a colorless solid: yield 2.0 g (64%); mp 59-61° C.; R_{F} 0.43 (dichloromethane:methanol 95:5); ¹H NMR δ 0.88 (t, 6H, J=7 Hz, 2×Me), 1.26-1.32 (br s, 44H, 22×CH₂), 1.53 (pentet, 4H, J=7 Hz, 2 OCH₂CH₂), 2.99 (s, 3H, NCH₃), 3.05 (s, 3H, NCH₃), 3.39 (t, 4H, J=6.5 Hz, decyl OCH₂), 3.50 (s, 4H, OCH₂C), 6.37 (d, 2H, J=15.5 Hz, COCH═), 6.86 (d, 2H, J=15.5 Hz, ═CHC); ¹³C NMR δ 166.8 (C═O), 145.3 (CH═CHC), 121.9 (COCH═), 72.9 (CH₂CH₂OC), 71.9 (OCH₂C), 48.9 (q C), 37.5 (NCH₃), 35.8 (NCH₃), 32.1 (CH₂CH₂CH₃), 29.86, 29.82, 29.77, 29.68, 29.52 (5 tetradecyl CH₂), 26.4 (CH₂CH₂CH₂O), 22.8 (CH₂CH₃), 14.3 (Me); HR ESI MS m/z calcd for C₄₁H₇₈N₂O₄Na (M+Na) 685.5854. found 685.5814.

### Example 19A

### N,N,N′,N′-Tetramethyl-4,4-bis(octyloxymethyl)heptanediamide (19a)

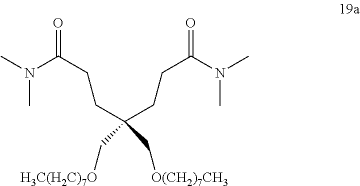

A mixture of compound 18a (Example 18A) (0.5 g, 1.0 mmol) and 10% Pd/C wet Degussa type catalyst (50 mg) in ethyl acetate (50 mL) is stirred under H₂ at atmospheric pressure for 24 h. The reaction mixture is filtered using a Celite™ bed and the filtrate is concentrated to give compound 19a as a colorless liquid: yield 0.46 g (91%); R_{F} 0.5 (dichloromethane:methanol 95:5); ¹H NMR δ 0.88 (t, 6H, J=6.5 Hz, 2×Me), 1.25-1.31 (br s, 20H, 10×CH₂), 1.50 (pentet, 4H, J=7 Hz, 2 OCH₂CH₂), 1.61 (4H, AA′XX′ pattern, CCH₂), 2.32 (4H, AA′XX′ pattern, COCH₂), 2.99 (s, 3H, NCH₃), 3.07 (s, 3H, NCH₃), 3.22 (s, 4H, OCH₂C), 3.34 (t, 4H, J=6.5 Hz, decyl OCH₂); ¹³C NMR δ 173.8 (C═O), 73.9 (CH₂CH₂OC), 71.7 (OCH₂C), 40.6 (qC), 37.4 (NCH₃), 35.6 (NCH₃), 32.0 (CH₂CH₂CH₃), 29.90, 29.64, 29.48 (3 octyl CH₂), 28.04, 27.94 (COCH₂CH₂, COCH₂), 26.5 (CH₂CH₂CH₂O), 22.8 (CH₂CH₃), 14.3 (Me); HR ESI MS m/z calcd for C₂₉H₅₈N₂O₄Na (M+Na) 521.4289. found 521.4278.

### Example 19B

### 4,4-Bis(decyloxymethyl)-N,N,N′,N′-tetramethyl-2,5-heptanediamide (19b)

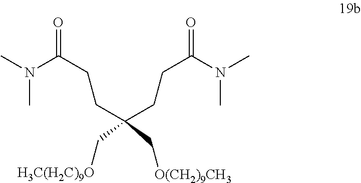

Hydrogenation of compound 18b (Example 18B) (1.0 g, 1.8 mmol) following the procedure of Example 19A gives the title compound as a colorless liquid: yield 0.90 g (89%); R_{F} 0.39 (dichloromethane:methanol 95:5); ¹H NMR δ 0.88 (t, 6H, J=7 Hz, 2×Me), 1.25-1.31 (br s, 28H, 14×CH₂), 1.51 (pentet, 4H, J=7 Hz, 2 OCH₂CH₂), 1.61 (4H, AA′XX′ pattern, CCH₂), 2.32 (4H, AA′XX′ pattern, COCH₂), 2.92 (s, 3H, NCH₃), 3.00 (s, 3H, NCH₃), 3.22 (s, 4H, OCH₂C), 3.34 (t, 4H, J=6.5 Hz, decyl OCH₂); ¹³C NMR δ 173.8 (C═O), 73.9 (CH₂CH₂OC), 71.7 (OCH₂C), 40.6 (q C), 37.4 (NCH₃), 35.6 (NCH₃), 32.0 (CH₂CH₂CH₃), 29.90, 29.82, 29.77, 29.67, 29.51 (5 decyl CH₂), 28.04, 27.94 (COCH₂CH₂, COCH₂), 26.5 (CH₂CH₂CH₂O), 22.8 (CH₂CH₃), 14.3 (Me); HR ESI MS m/z calcd for C₃₃H₆₆N₂O₄Na 577.4915. found 577.4918.

### Example 19C

### 4,4-Bis(dodecyloxymethyl)-N,N,N′,N′-tetramethyl-2,5-heptanediamide (19c)

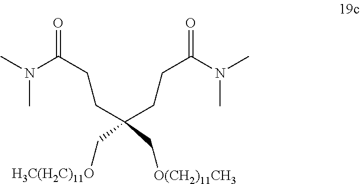

Hydrogenation of compound 18c (Example 18C) (0.70 g, 1.2 mmol) following the procedure of Example 19A gives compound 19c as a colorless solid: yield 0.65 g (92%); mp 46-48° C.; R_{F} 0.42 (dichloromethane:methanol 95:5); ¹H NMR δ 0.88 (t, 6H, J=7 Hz, 2×Me), 1.25-1.32 (br s, 36H, 18×CH₂), 1.52 (pentet, 4H, J=7 Hz, 2 OCH₂CH₂), 1.62 (4H, AA′XX′ pattern, CCH₂), 2.33 (4H, AA′XX′ pattern, COCH₂), 2.92 (s, 3H, NCH₃), 3.00 (s, 3H, NCH₃), 3.22 (s, 4H, OCH₂C), 3.34 (t, 4H, J=6.5 Hz, decyl OCH₂); ¹³C NMR δ 173.8 (C═O), 73.9 (CH₂CH₂OC), 71.7 (OCH₂C), 40.6 (q C), 37.4 (NCH₃), 35.5 (NCH₃), 32.1 (CH₂CH₂CH₃), 29.90, 29.83, 29.70, 29.52 (7 dodecyl CH₂), 28.05, 27.94 (COCH₂CH₂, COCH₂), 26.5 (CH₂CH₂CH₂O), 22.8 (CH₂CH₃), 14.3 (Me); HR ESI MS m/z calcd for C₃₇H₇₄N₂O₄Na (M+Na) 633.5541. found 633.5562.

### Example 19D

### N,N,N′,N′-Tetramethyl-4,4-bis(tetradecyloxymethyl)heptanediamide (19d)

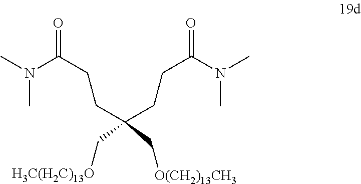

Hydrogenation of compound 18d (Example 18D) (1.60 g, 2.4 mmol) following the procedure of Example 19A gives the title compound as a colorless solid: yield, 1.50 g (94%); mp 47-49° C.; R_{F} 0.43 (dichloromethane:methanol 95:5); ¹H NMR S 0.88 (t, 6H, J=7 Hz, 2×Me), 1.25-1.32 (br s, 36H, 18×CH₂), 1.50 (pentet, 4H, J=7 Hz, 2 OCH₂CH₂), 1.62 (4H, AA′XX′ pattern, CCH₂), 2.33 (4H, AA′XX′ pattern, COCH₂), 2.92 (s, 3H, NCH₃), 3.00 (s, 3H, NCH₃), 3.22 (s, 4H, OCH₂C), 3.33 (t, 4H, J=6.5 Hz, decyl OCH₂); ¹³C NMR δ 173.8 (C═O), 74.0 (CH₂CH₂OC), 71.7 (OCH₂C), 40.6 (q C), 37.4 (NCH₃), 35.5 (NCH₃), 32.1 (CH₂CH₂CH₃), 29.87, 29.84, 29.70, 29.53 (9 tetradecyl CH₂), 28.04, 27.94 (COCH₂CH₂, COCH₂), 26.5 (CH₂CH₂CH₂O), 22.9 (CH₂CH₃), 14.4 (Me); HR ESI MS m/z calcd for C₄₁H₈₂N₂O₄Na (M+Na) 689.6167. found 689.6152.

### Example 20A

### N,N,N′,N′-Tetramethyl-4,4-bis(octyloxymethyl)-1,7-heptanediamine (20a)

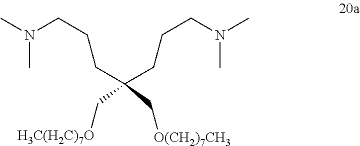

Diamide 19a (Example 19A) (0.6 g, 1.2 mmol) is added dropwise to a stirred suspension of LiAlH₄ (0.18 g, 4.8 mmol) in THF at 0° C. The reaction mixture is stirred at rt for 6 h, then the excess of LiAlH₄ is decomposed by dropwise addition of ethyl acetate (50 mL), water (0.3 mL), then 1M NaOH (0.3 mL) at 10° C. The mixture is filtered on a bed of Celite™, which is then washed with hot ethyl acetate. The combined filtrate and washings are dried (Na₂SO₄), then concentrated to a residue that is purified by flash column chromatography. Elution using a gradient of 5 to 15% MeOH in dichloromethane gives the title compound as a light brown liquid: yield 0.45 g (80%); R_{F} on basic alumina 0.71 (dichloromethane:methanol 94:6); ¹H NMR (CDCl₃) δ 0.88 (t, 6H, J=7.0 Hz, CH₃), 1.21-1.33 (m, 24H, 10 octyl CH₂, 2 CCH₂), 1.37-1.43 (m, 4H, NCH₂CH₂), 1.51 (pentet, 4H, J=7 Hz, OCH₂CH₂), 2.201 (t, 4H, J=7.5 Hz, NCH₂), 2.206 (s, 12H, NCH₃), 3.17 (s, 4H, OCH₂), 3.33 (t, 4H, J=6.5 Hz, OCH₂); ¹³C NMR (CDCl₃) δ 73.5 (CCH₂O), 71.5 (OCH₂), 60.9 (NCH₂), 45.7 (NCH₃), 41.0 (qC), 32.0 (CH₂CH₂CH₃), 29.85, 29.76, 29.65, 29.50 (3 octyl CH₂, CCH₂), 26.5 (OCH₂CH₂CH₂), 22.8 (CH₂CH₃), 21.45 (NCH₂CH₂), 14.3 (Me); HR ESI MS m/z calcd for C₂₉H₆₃N₂O₂ (M+H) 471.4884. found 471.4885.

### Example 20B

### 4,4-Bis(decyloxymethyl)-N,N,N′,N′-tetramethyl-1,7-heptanediamine (20b)

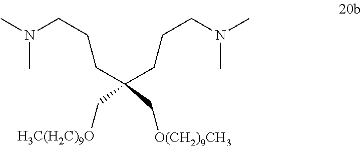

Diamide 19b (Example 19B) (0.90 g, 1.6 mmol) is reacted with LiAlH₄ (0.3 g, 8 mmol) following the procedure of Example 20A to give the title compound as a light brown liquid: yield 0.60 g (70%); R_{F} on basic alumina 0.52 (DCM:methanol 95:5); ¹H NMR (CDCl₃) δ 0.88 (t, 6H, J=7.0 Hz, CH₃), 1.21-1.33 (m, 32H, 14 decyl CH₂, 2 CCH₂), 1.39-1.44 (m, 4H, NCH₂CH₂), 1.51 (pentet, 4H, J=7 Hz, 2×OCH₂CH₂), 2.202 (t, 4H, J=7.5 Hz, NCH₂), 2.205 (s, 12H, NCH₃), 3.17 (s, 4H, OCH₂), 3.33 (t, 4H, J=6.5 Hz, OCH₂); ¹³C NMR (CDCl₃) δ 73.5 (CCH₂O), 71.53 (OCH₂), 60.9 (NCH₂), 45.7 (NCH₃), 41.0 (qC), 32.1 (CH₂CH₂CH₃), 29.85, 29.77, 29.70, 29.52 (5 decyl CH₂, CCH₂), 26.5 (OCH₂CH₂CH₂), 22.8 (CH₂CH₃), 21.4 (NCH₂CH₂), 14.3 (Me); HR ESI MS m/z calcd for C₃₃H₇₁N₂O₂ (M+H) 527.5510. found 527.5502.

### Example 20C

### 4,4-Bis(dodecyloxymethyl)-N,N,N′,N′-tetramethyl-1,7-heptanediamine (20c)

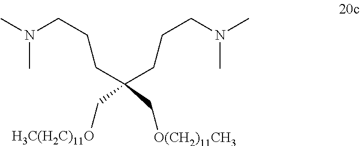

Diamide 19c (Example 19C) (0.64 g, 1.0 mmol) is reacted with LiAlH₄ (0.16 g, 4.2 mmol) following the procedure of Example 20A to give the title compound as a light brown liquid: yield 0.55 g (90%); R_{F} on basic alumina 0.7 (DCM:methanol 93:7); ¹H NMR (CDCl₃) δ 0.88 (t, 6H, J=7.0 Hz, CH₃), 1.21-1.33 (m, 40H, 18 dodecyl CH₂, 2 CCH₂), 1.37-1.43 (m, 4H, NCH₂CH₂), 1.51 (pentet, 4H, J=7 Hz, 2×OCH₂CH₂), 2.186 (t, 4H, J=7.5 Hz, NCH₂), 2.189 (s, 12H, NCH₃), 3.17 (s, 4H, OCH₂), 3.33 (t, 4H, J=6.5 Hz, OCH₂); ¹³C NMR (CDCl₃) δ 73.5 (CCH₂O), 71.5 (OCH₂), 60.9 (NCH₂), 45.7 (NCH₃), 40.9 (qC), 32.1 (CH₂CH₂CH₃), 29.85, 29.82, 29.74, 29.52 (7 dodecyl CH₂, CCH₂), 26.5 (OCH₂CH₂CH₂), 22.8 (CH₃CH₂), 21.4 (NCH₂CH₂), 14.3 (Me); HR ESI MS m/z calcd for C₃₇H₇₉N₂O₂ (M+H) 583.6136. found 583.6123.

### Example 20D

### N,N,N′,N′-Tetramethyl-4,4-bis(tetradecyloxymethyl)-1,7-heptanediamine (20d)

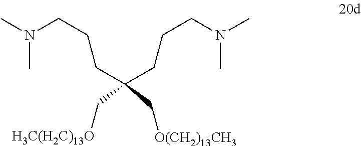

Diamide 19d (Example 19D) (1.0 g, 1.5 mmol) is reacted with LiAlH₄ (0.23 g, 6.0 mmol) following the procedure of Example 20A to give the title compound as a light brown liquid: yield 0.60 g (68%); R_{F} on basic alumina 0.52 (dichloromethane:methanol 96:4); ¹H NMR (CDCl₃) δ 0.88 (t, 6H, J=7.0 Hz, CH₃), 1.21-1.35 (m, 48H, 22 tetradecyl CH₂, 2 CCH₂), 1.38-1.43 (m, 4H, NCH₂CH₂), 1.51 (pentet, 4H, J=7 Hz, 2×OCH₂CH₂), 2.209 (t, 4H, J=7.5 Hz, NCH₂), 2.211 (s, 12H, NCH₃), 3.17 (s, 4H, OCH₂), 3.33 (t, 4H, J=6.5 Hz, OCH₂); ¹³C NMR (CDCl₃) δ 73.5 (CCH₂O), 71.5 (OCH₂), 60.9 (NCH₂), 45.6 (NCH₃), 40.9 (qC), 32.1 (CH₂CH₂CH₃), 29.87, 29.77, 29.71, 29.52 (9 tetradecyl CH₂, CCH₂), 26.5 (OCH₂CH₂CH₂), 22.9 (CH₂CH₃), 21.4 (NCH₂CH₂), 14.3 (Me); HR ESI MS m/z calcd for C₄₁H₈₇N₂O₂ (M+1) 639.6762. found 639.6744.

### Example 21A

### N,N,N,N′,N′,N′-Hexamethyl-4,4-bis(octyloxymethyl)-1,7-heptanediammonium diiodide (21a)

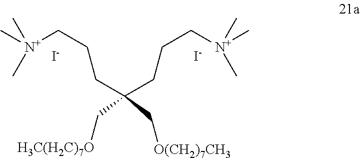

Methyl iodide (1.51 g, 10.6 mmol) is added to a stirred solution of amine 20a (Example 20A) (0.5 g, 1.0 mmol) in THF (30 mL). The reaction mixture is refluxed for 12 h, then concentrated. The solid residue is purified by flash column chromatography using 10% methanol in dichloromethane as eluent to give the title compound as an off-white solid, yield: 0.75 g (94%); mp 233-236° C.; R_{F} 0.5 on basic alumina (7% methanol in dichloromethane); ¹H NMR (CDCl₃) δ 0.88 (t, 6H, J=7.0 Hz, CH₃), 1.23-1.31 (m, 20H, 10×CH₂), 1.43 (m, 4H, J=8.2 Hz NCH₂CH₂CH₂), 1.51 (pentet, 4H, J=6.5 Hz, 2×OCH₂CH₂), 1.88 (m, 4H, NCH₂CH₂), 3.21 (s, 4H, OCH₂), 3.36 (t, 4H, J=6.5 Hz, OCH₂), 3.44 (s, 18H, NCH₃), 3.73 (m, 2H, NCH₂); ¹³C NMR (CDCl₃) δ 72.5 (CCH₂O), 71.6 (OCH₂), 67.8 (NCH₂), 54.5 (NCH₃), 41.4 (qC), 32.0 (CH₂CH₂CH₃), 29.82, 29.62, 29.53 (octyl CH₂), 26.7 (CCH₂), 26.5 (OCH₂CH₂CH₂), 22.8 (CH₃CH₂), 17.8 (NCH₂CH₂), 14.3 (Me); HR ESI MS m/z calcd for C₃₁H₆₈IN₂O₂ (M−I) 627.4320. found 627.4267.

### Example 21B

### 4,4-Bis(decyloxymethyl)-N,N,N,N′,N′,N′-hexamethyl-1,7-heptanediammonium diiodide (21b)

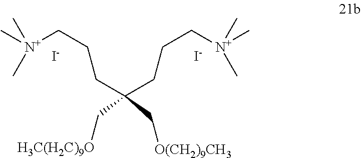

Alkylation of amine 20b (Example 20B) (0.5 g, 0.9 mmol) with methyl iodide (1.34 g, 9.43 mmol) following the procedure of Example 21A gives the title product as an off-white solid: yield 0.70 g (91%); mp 240-243° C.; R_{F} 0.5 on basic alumina (7% methanol in dichloromethane); ¹H NMR (CDCl₃) δ 0.88 (t, 6H, J=7.0 Hz, CH₃), 1.23-1.33 (m, 28H, 14×CH₂), 1.43 (m, 4H, NCH₂CH₂CH₂), 1.51 (pentet, 4H, J=6.5 Hz, 2×OCH₂CH₂), 1.88 (m, 4H, NCH₂CH₂), 3.22 (s, 4H, OCH₂), 3.37 (t, 4H, J=6.5 Hz, OCH₂), 3.45 (s, 18H, NCH₃), 3.72 (m, 2H, NCH₂); ¹³C NMR (CDCl₃) δ 72.5 (CCH₂O), 71.6 (OCH₂), 67.8 (NCH₂), 54.4 (NCH₃), 41.4 (qC), 32.1 (CH₂CH₂CH₃), 29.88, 29.79, 29.67, 29.50 (5 decyl CH₂), 26.7 (CCH₂), 26.5 (OCH₂CH₂CH₂), 22.8 (CH₃CH₂), 17.9 (NCH₂CH₂), 14.3 (Me); HR ESI MS m/z calcd for C₃₅H₇₆IN₂O₂ (M−I) 683.4946. found 683.4895.

### Example 21C

### 4,4-Bis(dodecyloxymethyl)-N,N,N,N′,N′,N′-hexamethyl-1,7-heptanediammonium diiodide (21c)

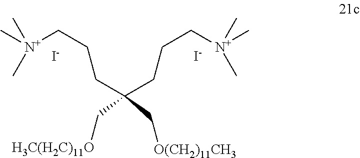

Alkylation of amine 20c (Example 20C) (0.50 g, 0.8 mmol) with methyl iodide (1.2 g, 8.4 mmol) following the procedure of Example 21A gives the title product as an off-white solid: yield 0.65 g (88%); mp 252-254° C.; R_{F} 0.5 on basic alumina (7% methanol in dichloromethane); ¹H NMR (CDCl₃) δ 0.88 (t, 6H, J=7.0 Hz, CH₃), 1.22-1.34 (m, 20H, 10×CH₂), 1.41 (m, 4H, NCH₂CH₂CH₂), 1.51 (pentet, 4H, J=6.5 Hz, 2×OCH₂CH₂), 1.87 (m, 4H, NCH₂CH₂), 3.22 (s, 4H, OCH₂), 3.37 (t, 4H, J=6.5 Hz, OCH₂), 3.46 (s, 18H, NCH₃), 3.71 (m, 4H, 2H, NCH₂); ¹³C NMR (CDCl₃) δ 72.5 (CCH₂O), 71.6 (OCH₂), 67.8 (NCH₂), 54.4 (NCH₃), 41.3 (qC), 32.1 (CH₂CH₂CH₃), 29.86, 29.82, 29.69, 29.51 (decyl CH₂), 26.72, 26.48 (OCH₂CH₂CH₂), 22.83 (CH₃CH₂), 17.8 (NCH₂CH₂), 14.3 (Me); HR ESI MS m/z calcd for C₃₉H₈₄IN₂O₂ (M−I) 739.5572. found 739.5548.

### Example 21D

### N,N,N,N′,N′,N′-Hexamethyl-4,4-bis(tetradecyloxymethyl)-1,7-heptanediammonium diiodide (21d)

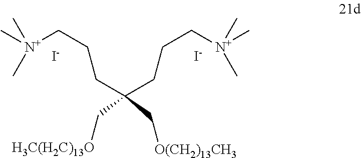

Alkylation of amine 20d (Example 20D) (0.5 g, 0.9 mmol) with methyl iodide (0.89 g, 6.2 mmol) following the procedure of Example 21A gives the title product as an off-white solid: yield 0.51 g (88%); mp 238-241° C.; R_{F} 0.47 on basic alumina (8% methanol in dichloromethane); ¹H NMR (CDCl₃) δ 0.88 (t, 6H, J=7.0 Hz, CH₃), 1.22-1.32 (m, 44H, 22×CH₂), 1.44 (m, 4H, NCH₂CH₂CH₂), 1.51 (pentet, 4H, J=6.5 Hz, 2×OCH₂CH₂), 1.89 (m, 4H, NCH₂CH₂), 3.22 (s, 4H, OCH₂), 3.37 (t, 4H, J=6.5 Hz, OCH₂), 3.44 (s, 18H, NCH₃), 3.74 (m, 4H, NCH₂); ¹³C NMR (CDCl₃) δ 72.4 (CCH₂O), 71.6 (OCH₂), 67.8 (NCH₂), 54.5 (NCH₃), 41.3 (qC), 32.1 (OCH₂CH₂), 29.87, 29.82, 29.70, 29.52 (decyl CH₂), 26.61, 26.48 (OCH₂CH₂CH₂), 22.8 (CH₃CH₂), 17.9 (NCH₂CH₂), 14.3 (Me); HR ESI MS m/z calcd for C₄₃H₉₂IN₂O₂, 795.6198. found 795.6215.

### Example 22A

### Sodium 2,2-bis(butyloxymethyl)-1,3-propanediol disulfate (22a)

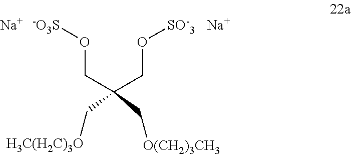

NaH (60% oil dispersion, washed with hexanes, 7.15 g, 0.18 mol, 7.2 eq) is added to an ice cold DMF (100 mL) solution of 1-butanol (4.15 g, 0.056 mol, 2.2 eq), and the reaction mixture is stirred for 1.5 h. A solution of pentaerythritol bicyclic sulfate (XIII, Scheme 5, prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386) (6.40 g, 0.025 mol) in DMF (50 mL) is added, followed, after 2 h, by an additional aliquot of NaH (60% oil dispersion, washed with hexanes, 2.06 g, 0.052 mol, 2.0 eq). After being stirred for 24 h at 60° C., the reaction mixture is cooled to 0° C. and MeOH is added slowly dropwise until foaming ceased. 20% HCl is then added dropwise to neutralize (pH paper) the mixture. The reaction mixture is concentrated to give a brown solid which is recrystallized from 1:19 water/MeOH to give the title compound (22a, 7.34 g, 0.016 mol, 65%): recrystallized from methanol-ethyl acetate as colorless opaque needles: mp 200-203° C., becomes transparent, 225° C. starts turning brown, 245-250° C.; ¹H NMR (DMSO-d₆) δ 3.70 (s, 4H, —OSO₃CH₂C), 3.33 (t, 4H, J=6.5 Hz, OCH₂CH₂), 3.29 (s, 4H, CCH₂), 1.44 (p, 4H, J=6.9 Hz, OCH₂CH₂), 1.32 (sextet, 4H, J=7.3 Hz, CH₂CH₂CH₃), 0.86 (t, 6H, J=7.4 Hz, CH₃); ¹³C NMR δ 70.5 (OCH₂CH₂), 69.4 (CCH₂), 65.1 (—OSO₃CH₂), 43.4 (q C), 31.3 (CH₃CH₂CH₂), 18.4 (CH₃CH₂), 13.8 (CH₃); ESI MS m/z calc for C₁₃H₂₇O₁₀S₂: 407.10. found: 406.9; for C₁₃H₂₆O₁₀S₂Na: 429.09. found: 429.3; for (C₁₃H₂₆O₁₀S₂)/2: 203.05. found: 203.1. Anal. Calc. for C₁₃H₂₈O₁₀S₂Na₂.H₂O: C, 33.19; H, 6.00. Found: C, 32.97; H, 5.77.

### Example 22B

### Sodium 2,2-bis(hexyloxymethyl)-1,3-propanediol disulfate (22b)

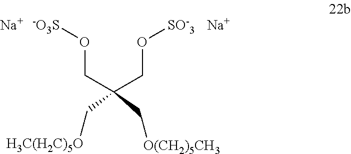

A hexanes-washed 60% oil dispersion of NaH (5.20 g, 0.13 mol, 2.8 eq) is added to an ice cold DMF (200 mL) solution of 1-hexanol (4.68 g, 0.046 mol, 2.1 eq) then the mixture is stirred for 30 min. A DMF (25 mL) solution of the bicyclic sulfate XIII (Scheme 5, prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386) (5.60 g, 0.022 mol) is added to the ice cold reaction mixture, followed after 2 h, by an additional aliquot of hexanes-washed NaH (4.40 g, 0.11 mol, 2.4 eq). After being stirred for 48 h at rt, the reaction mixture is cooled to 0° C. and MeOH is added slowly dropwise until foaming ceased. 5% HCl is then added dropwise to neutralize (pH paper) the mixture. The mixture is then concentrated to give a yellow solid which is recrystallized with a 1:19 water/MeOH solvent system to give the title compound (6.63 g, 0.0215 mol, 61%). The analytical sample is recrystallized from methanol: mp 185-187° C.; ¹H NMR (DMSO-d₆) δ 3.70 (s, 4H, —OSO₃CH₂C), 3.32 (t, 4H, J=6.5 Hz, OCH₂CH₂), 3.29 (s, 4H, CCH₂), 1.46 (p, 4H, J=6.7 Hz, OCH₂CH₂), 1.23-1.32 (complex m, 12H, 2×(CH₂)₃), 0.86 (t, 6H, J=6.9 Hz, CH₃); ¹³C NMR δ 70.8 (OCH₂CH₂), 69.4 (CCH₂), 65.1 (—OSO₃CH₂), 43.4 (q C), 31.1 (CH₃CH₂CH₂), 29.1 (OCH₂CH₂), 25.3 (OCH₂CH₂CH₂), 22.1 (CH₃CH₂), 13.9 (CH₃); ESI MS m/z calc for C₁₇H₃₅O₁₀S₂: 463.17. found: 462.9; for C₁₇H₃₄O₁₀S₂Na: 485.15. found: 485.3; for (C₁₇H₃₄O₁₀S₂)/2: 231.08. found: 231.2. Anal. Calc. for C₁₇H₃₆O₁₀S₂Na₂.H₂O: C, 38.78; H, 6.89. Found: C, 38.29; H, 6.92.

### Example 22C

### Sodium 2,2-bis(octyloxymethyl)-1,3-propanediol disulfate (22c)

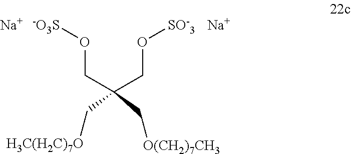

A 60% oil dispersion of sodium hydride (6.08 g, 6.6 eq, 0.152 mol) is washed exhaustively with dried hexanes then added to an ice cold DMF (200 mL) solution of 1-octanol (6.60 g, 2.2 eq, 0.0506 mol). The solution is left to stir under N₂ and in an ice bath for 20 min. A DMF solution (50 mL) of the bicyclic sulfate XIII (Scheme 5, prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386) (6.00 g, 0.0230 mol) is added slowly dropwise to the ice cold mixture over a 15 min period. The mixture is then allowed to slowly reach room temperature and left to stir for 24 h. MeOH is added dropwise to an ice cold reaction mixture until no more gas is formed. This is followed by dropwise addition of 20% H₂SO₄ until a neutral (pH paper) solution is obtained. Concentration gives a yellow viscous liquid which is extracted with boiling MeOH (˜1.5 L). A concentrated extract is allowed to undergo water promoted crystallization. The title compound (9.40 g, 72.3%) is a white powder with complex melting behavior: at 37.3° C. softening occurs and the sample becomes slightly transparent, at 108.0° C., it solidifies and becomes more opaque, at 159.8° C., it becomes increasingly opaque, at 167.1° C., it softens and becomes transparent, at 184.9° C., it melts; R_{F} 0.53 (butanol:water:methanol 10:5:2); ¹H NMR (DMSO-d₆) δ 3.71 (s, 4H, —OSO₃CH₂), 3.31 (t, 4H, J=6.5 Hz, OCH₂CH₂), 3.29 (s, 4H, CCH₂), 1.46 (p, 4H, J=7.0 Hz, OCH₂CH₂), 1.22-1.31 (complex m, 20H, 2×(CH₂)₅), 0.85 (t, 6H, J=7.0 Hz, CH₃); ¹³C NMR δ 70.8 (OCH₂CH₂), 69.3 (CCH₂), 65.3 (—OSO₃CH₂), 43.4 (q C), 31.3 (CH₃CH₂CH₂), 29.2 (OCH₂CH₂), 28.9, 28.7 (2 alkyl C), 25.7 (OCH₂CH₂CH₂), 22.1 (CH₃CH₂), 14.0 (CH₃); ESI MS m/z calc for C₂₁H₄₃O₁₀S₂: 519.23. found: 519.1; for C₂₁H₄₂O₁₀S₂Na: 541.21. found 541.3; for (C₂₁H₄₂O₁₀S₂)/2: 259.11. found: 259.3. Anal. Calc. for C₂₁H₃₂O₁₀S₂Na₂: C, 44.67; H, 7.50. Found: C, 44.18; H, 7.84.

### Example 22D

### Sodium 2,2-bis(decyloxymethyl)-1,3-propanediol disulfate (22d)

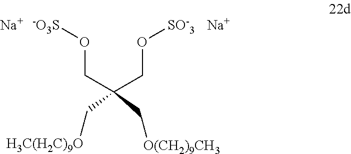

A 60% oil dispersion of sodium hydride (5.53 g, 6.6 eq, 0.138 mol) is washed exhaustively with dried hexanes then added to an ice cold solution of 1-decanol (6.60 g, 2.2 eq, 0.0461 mol) in DMF (200 mL). The mixture is left to stir in an ice bath under N₂ for 20 min and then a solution of the bicyclic sulfate (XIII, Scheme 5, prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386) (5.45 g, 0.0209 mol) in DMF (50 mL) is added dropwise over 30 min. The mixture is then allowed to reach room temperature and left to stir for 24 hrs. The reaction mixture is cooled in an ice bath, then methanol is added dropwise until gas evolution ceased. The solution is neutralized with 20% H₂SO₄ (pH paper). Concentration gives a yellow viscous liquid which is extracted with boiling MeOH (˜1.5 L). A concentrated extract is allowed to undergo water promoted crystallization. The title compound (7.60 g, 58.4%) is a colourless powder. Complex melting behavior is observed: at 38.0° C., it softens and becomes slightly transparent, at 62.0° C., it becomes a white translucent paste, at 105.0° C., an opaque white liquid, and at 196.0° C., it melts; R_{F} 0.55 (butanol:water:methanol 10:5:2); ¹H NMR (DMSO-d₆) δ 3.70 (s, 4H, —OSO₃CH₂C), 3.31 (t, 4H, J=6.5 Hz, OCH₂CH₂), 3.28 (s, 4H, CCH₂), 1.46 (quintet, 4H, J=6.5 Hz, OCH₂CH₂), 1.21-1.33 (complex m, 28H, 2×(CH₂)₇), 0.85 (t, 6H, J=6.5 Hz, CH₃); ¹³C NMR δ 70.8 (OCH₂CH₂), 69.4 (CCH₂O), 65.3 (⁻OSO₃CH₂), 43.4 (q C), 31.3 (CH₃CH₂CH₂), 29.16 (OCH₂CH₂), 29.12, 29.03, 28.92, 28.74 (4 alkyl C), 25.7 (OCH₂CH₂CH₂), 22.1 (CH₃CH₂), 14.0 (CH₃); ESI MS m/z calc for C₂₅H₅₁O₁₀S₂: 575.29. found: 575.1; for C₂₅H₅₀O₁₀S₂Na: 597.27. found: 597.5; for (C₂₅H₅₀O₁₀S₂)/2: 287.14. found: 287.3. Anal. Calc. for C₂₅H₅₀O₁₀S₂Na₂: C, 48.38; H, 8.12. Found: C, 48.37; H, 8.15.

### Example 22E

### Sodium 2,2-bis(dodecyloxymethyl)-1,3-propanediol disulfate (22e)

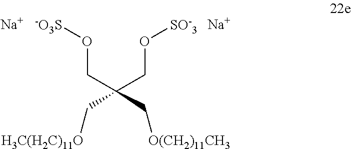

A 60% oil dispersion of sodium hydride (2.20 g, 10.5 eq, 0.093 mol) is washed exhaustively with dried hexanes then added to an ice cold solution of 1-dodecanol (3.49 g, 2.1 eq, 0.0186 mol) in DMF (50 mL). The flask is flushed with N₂ and the solution is left to stir in an ice bath for 2 h. A solution (10 mL) of the bicyclic sulfate (XIII, Scheme 5, prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386) (2.35 g, 0.0209 mol) in DMF (10 mL) is added to the ice cold mixture. The mixture is allowed to reach room temperature and left to stir for 5 h before additional NaH oil dispersion (1.90 g) is added. After 48 h, MeOH is added dropwise to an ice cold reaction mixture until gas evolution ceased. The reaction mixture is neutralized with 20% H₂SO₄ (pH paper). Concentration gives a yellow viscous liquid which is extracted with boiling MeOH (˜1.5 L). The solid residue obtained after removal of the MeOH is dissolved in water (100 mL) and extracted with CH₂Cl₂ (50 mL). The water layer is concentrated to give a white solid that is crystallized from 10% H₂O in MeOH. The title compound (2.10 g, 14%) is collected as a colourless powder: complex melting behavior is observed; at 52.0° C., it softens, at 102.6° C., it becomes a slightly transparent solid, at 116.2° C., a colourless opaque fluid, at 141.3° C., an increasingly transparent semi-solid, at 181.3° C., it melts; R_{F} 0.58 (butanol:water:methanol 10:5:2); ¹H NMR (DMSO-d₆) δ 3.69 (s, 4H, —OSO₃CH₂), 3.31 (t, 4H, J=6.5 Hz, OCH₂CH₂), 3.28 (s, 4H, CCH₂), 1.46 (p, 4H, J=6.5 Hz, OCH₂CH₂), 1.21-1.32 (complex m, 36H, 2×(CH₂)₉), 0.85 (t, 6H, J=7.0 Hz, CH₃); ¹³C NMR δ 70.8 (OCH₂CH₂), 69.4 (CCH₂), 65.3 (—OSO₃CH₂), 43.4 (q C), 31.4 (CH₃CH₂CH₂), 30.7, 29.2, 29.2, 29.2, 29.1, 29.9, 29.0, 28.8 (alkyl CH₂), 25.7 (OCH₂CH₂CH₂), 22.2 (CH₃CH₂), 14.0 (CH₃); ESI MS m/z calc for C₂₉H₅₉O₁₀S₂: 631.35, found: 631.2; for C₂₉H₅₈O₁₀S₂Na: 653.34. found: 553.5; for (C₂₉H₅₈O₁₀S₂)/2: 315.17. found: 315.3. Anal. Calc. for C₂₉H₅₈O₁₀S₂Na₂: C, 51.46; H, 8.54. Found: C, 51.40; H, 8.76.

### Example 22F

### Sodium 2,2-bis(tetradecyloxymethyl)-1,3-propanediol disulfate (22f)

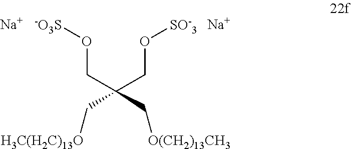

NaH (4.70 g, 60% oil dispersion, 6.6 eq, 0.1175 mol) is washed exhaustively with dried hexanes then added to an ice cold solution of 1-tetradecanol (8.40 g, 2.2 eq, 0.0392 mol) in DMF (200 mL). The solution is flushed with N₂ and left to stir in an ice bath for about 20 min. A DMF solution (50 mL) of the bicyclic sulfate (XIII, Scheme 5, prepared by the methods of Gulyás, H.; Dobó, A.; Bakos, J. Can. J. Chem. 2001, 79, 1040-1048 and/or Gulyás, H.; Árva, P.; Bakos, J. Chem. Commun. 1997, 2385-2386) (4.63 g, 0.0178 mol) is added slowly dropwise to the ice cold mixture over a 30 min period and then the temperature is allowed to rise slowly to rt. The reaction is monitored by recording NMR spectra of aliquots using singlets at ˜5.00 ppm and ˜4.70 ppm to monitor bicyclic sulfate and monoproduct, respectively. If reaction is still incomplete after 72 h, the reaction mixture is warmed to 45° C. for 2 h before being quenched by dropwise addition of MeOH. 20% H₂SO₄ is added to neutralize the reaction mixture. Concentration gives a colourless viscous liquid which is extracted with boiling MeOH (1.5 L). The extract is concentrated. On trituration with water, the residue solidified to a white powder (9.7 g, 74.6%): mp 195° C.; R_{F} 0.60 (butanol:water:methanol 10:5:2); ¹H NMR (DMSO-d6) δ 3.71 (s, 4H, —OSO₃CH₂), 3.31 (t, 4H, J=6.5 Hz, OCH₂CH₂), 3.29 (s, 4H, CCH₂O), 1.47 (p, 4H, J=6.5 Hz, 2 OCH₂CH₂), 1.21-1.32 (complex m, 44H, 2×11CH₂), 0.85 (t, 6H, J=7.0 Hz, 2 CH₃); ¹³C NMR δ 70.7 (OCH₂CH₂), 69.3 (CCH₂O), 65.2 (—OSO₃CH₂), 43.3 (q C), 31.3 (CH₃CH₂CH₂), 29.14, 29.11, 29.09, 29.05, 29.04, 28.92, 28.72 (alkyl C), 25.7 (OCH₂CH₂CH₂), 22.1 (CH₃CH₂), 13.9 (CH₃); ESI MS m/z calc for C₃₃H₆₆O₁₀S₂Na: 709.40. found: 709.6. Anal. Calc. for C₃₃H₆₆O₁₀S₂Na₂: C, 54.08; H, 9.08. Found: C, 53.72; H, 9.00.

### Example 23A

### Sodium 5,5-bis(octyloxymethyl)-3,7-dioxa-1,9-nonanediol disulfate (23a)

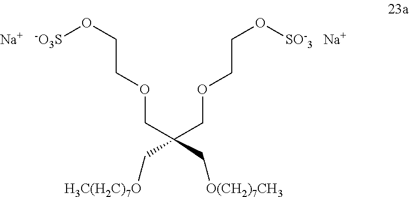

To a stirred ice-bath cooled solution of 2,2-bis(octyloxymethyl)-1,3-propanediol 2a (Example 2A) (5.40 g, 0.015 mol) in THF (100 mL) is added a hexanes-washed 60% oil dispersion of sodium hydride (2.60 g, 0.065 mol, 4.3 eq). The cooled reaction mixture is stirred for 30 min, then a solution of ethylene sulfate (prepared by the method of Baker, W.; Field, F. B. J. Chem. Soc. 1932, 86-91 and/or Brimacombe, J. S.; Foster, A. B.; Hancock, E. B.; Overend, W. G.; Stacey, M. J. Chem. Soc. 1960, 201-211) (4.0 g, 0.032 mol, 2.2 eq) in THF (50 mL) is added dropwise to the reaction mixture over a 20 min period. The reaction mixture is stirred for 19 h at rt, then MeOH is added carefully dropwise until foaming ceased. 10% HCl is added to neutralize the reaction (pH paper). Concentration gives a white paste that is purified using column chromatography with a solvent gradient changing from pure EtOAc to MeOH:EtOAc 2:3. The product is obtained as a white solid (6.87 g, 70.2%): mp 143.0-145.5° C., ¹H NMR (500.13 MHz, MeOD:CDCl₃, 50:50) δ 0.89 (t, J=6.8 Hz, 6H, 2×CH₃), 1.31 (br m, 20H, alkyl protons), 1.54 (quintet, J=7.0 Hz, 4H, 2×OCH₂CH₂), 3.38 (s, 4H, CH₂CH₂CH₂OCH₂C), 3.39 (t, J=6.5 Hz, 4H, 2×OCH₂CH₂), 3.49 (s, 4H, O₃SOCH₂CH₂OCH₂C), 3.68 (XX′ part of AA′XX′ pattern, J_{AX}+J_{A′X}=10.0 Hz, 4H, 2×⁻O₃SOCH₂CH₂), 4.14 (AA′ part of AA′XX′ pattern, J_{AX}+J_{A′X}=10.0 Hz, 4H, 2×⁻O₃SOCH₂CH₂); ¹³C NMR (125.77 MHz, MeOD) δ 14.3 (2×CH₃), 23.2 (2×CH₂CH₃), 26.8 (2×OCH₂CH₂CH₂), 30.0, 30.1, 30.2 (2×OCH₂CH₂ and alkyl chain carbons), 32.5 (2×CH₂CH₂CH₃), 46.3 (q C), 67.6 (2×OSO₃CH₂), 70.1 (2×CH₂OCH₂CH₂CH₂), 70.7 (2×OSO₃CH₂CH₂), 71.1 (2×O₃SOCH₂CH₂OCH₂), 72.4 (2×OCH₂CH₂CH₂); ESI MS (neg ion mode): calc for C₂₅H₅₀O₁₂S₂Na (M−Na) 629.26. found 629.3; calc for (M−2Na)/2 303.14. found 303.3, calc for M−2Na+H, 607.28. found 607.1; calc for 2M−Na 1281.52. found 1281.1. Anal. Calc. for C₂₅H₅₀O₁₂S₂Na₂: C, 46.00; H, 7.72. Found: C, 46.11; H, 7.47.

### Example 23B

### Sodium 5,5-bis(decyloxymethyl)-3,7-dioxa-1,9-nonanediol disulfate (23b)

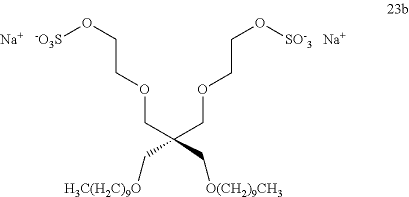

A solution of 2,2-bis(decyloxymethyl)-1,3-propanediol 2b (Example 2B) (6.86 g, 0.0166 mol) in THF (100 mL) is cooled in an ice-bath and then hexane-washed sodium hydride (60% oil dispersion, 2.20 g, 0.055 mol, 3.3 eq) is added. After the cooled reaction mixture has stirred for 30 min, a THF (50 mL) solution of ethylene sulfate (prepared by the method of Baker, W.; Field, F. B. J. Chem. Soc. 1932, 86-91 and/or Brimacombe, J. S.; Foster, A. B.; Hancock, E. B.; Overend, W. G.; Stacey, M. J. Chem. Soc. 1960, 201-211) (4.5 g, 0.036 mol, 2.2 eq) is added dropwise over a 20 min period. The cooling bath is removed and the reaction mixture is stirred for 24 hrs. MeOH is added carefully dropwise until foaming ceased. 10% HCl is added to neutralize the reaction (wet pH paper), then the reaction mixture is concentrated to a white paste. The product is crystallized from EtOAc:MeOH 75:25: yield; mp 165-172° C.; ¹H NMR (500.13 MHz, DMSO) δ 0.84 (t, J=6.5 Hz, 6H, 2×CH₃), 1.24 (br m, 28H, alkyl protons), 1.46 (quintet, J=7.2 Hz, 4H, 2×OCH₂CH₂), 3.26 (s, 4H, 2×CH₂CH₂CH₂OCH₂C), 3.38 (s, 4H, 2×O₃SOCH₂CH₂OCH₂C), 3.39 (t, J=6.6 Hz, 4H, 2×OCH₂CH₂), 3.47 (XX′ part of AA′XX′ pattern, J_{AX}+J_{A′X}=10.0 Hz, 4H, 2×⁻O₃SOCH₂CH₂), 3.77 (AA′ part of AA′XX′ pattern, J_{AX}+J_{A′X}=10.0 Hz, 4H, 2×⁻O₃SOCH₂CH₂); ¹³C NMR (125.77 MHz, MeOD) δ 13.9 (2×CH₃), 22.1 (2×CH₂CH₃), 25.6 (2×OCH₂CH₂CH₂), 28.7, 28.8, 29.0, 29.0, 29.1 (2×OCH₂CH₂ and 14 alkyl chain carbons), 31.3 (2×CH₂CH₂CH₃), 45.1 (q C), 64.8 (2×OSO₃CH₂), 68.9 (2×CH₂OCH₂CH₂CH₂), 69.6 (2×OSO₃CH₂CH₂), 70.0 (2×OSO₃CH₂CH₂OCH₂), 70.7 (2×OCH₂CH₂CH₂). Anal. Calc. for C₂₉H₅₈O₁₂S₂Na₂: C, 49.14, H, 8.25. Found: C, 49.36, H, 8.38.

### Example 23C

### Sodium 5,5-bis(dodecyloxymethyl)-3,7-dioxa-1,9-nonanediol disulfate (23c)

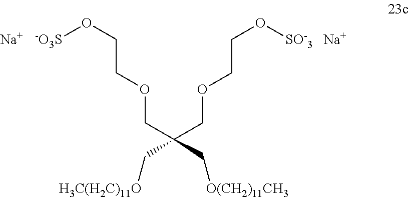

To an ice-bath cooled solution of 2,2-bis(dodecyloxymethyl)-1,3-propanediol 2c (Example 2C) (7.08 g, 0.015 mol) in THF (100 mL) is added a hexanes-washed 60% oil dispersion of NaH (2.20 g, 0.055 mol, 3.6 eq). The cooled reaction mixture is stirred for 30 min then a solution of ethylene sulfate (prepared by the method of Baker, W.; Field, F. B. J. Chem. Soc. 1932, 86-91 and/or Brimacombe, J. S.; Foster, A. B.; Hancock, E. B.; Overend, W. G.; Stacey, M. J. Chem. Soc. 1960, 201-211) (4.0 g, 0.032 mol, 2.2 eq) in THF (50 mL) is added dropwise over a 20 min period. After the reaction mixture has been stirred for 21 h at rt, not all starting material has been consumed (TLC). More ethylene sulfate (0.1 g, 0.8 mmol) is added and the reaction mixture is stirred for another 3 h. MeOH is added carefully dropwise until foaming ceases, then 10% HCl is added until the reaction mixture is neutral (pH paper). Concentration yields a white paste that is taken up in a 1:1 CH₂Cl₂/MeOH. The mixture is heated to a boil then filtered. The filtrate is then heated until the mixture became clear. The solution is allowed to cool to rt before being refrigerated. The product is collected by filtration (9.53 g, 0.0125 mol, 83.3%): mp 144.5-146.0° C.; ¹H NMR (500.13 MHz, MeOD) δ 0.90 (t, J=7.1 Hz, 6H, 2×CH₃), 1.29 (br m, 36H, alkyl protons), 1.54 (quintet, J=7.2 Hz, 4H, 2×OCH₂CH₂), 3.38 (s, 4H, 2×CH₂CH₂CH₂OCH₂C), 3.39 (t, J=6.6 Hz, 4H, 2×OCH₂CH₂CH₂), 3.47 (s, 4H, O₃SOCH₂CH₂OCH₂C), 3.64 (AA′ part of AA′XX′ pattern, J_{AX}+J_{A′X}=10.0 Hz, 4H, 2×⁻O₃SOCH₂CH₂), 4.09 (XX′ part of AA′XX′ pattern, J_{AX}+J_{A′X}=10.0 Hz, 4H, 2×⁻O₃SOCH₂CH₂); ¹³C NMR (125.77 MHz, MeOD) δ 14.6 (2×CH₃), 23.9 (2×CH₂CH₃), 27.6 (2×OCH₂CH₂CH₂), 30.6, 30.8, 30.9, 30.9, 31.0 (2×OCH₂CH₂ and 12 alkyl chain carbons), 33.2 (2×CH₂CH₂CH₃), 47.0 (q C), 68.3 (2×OSO₃CH₂), 70.7 (CH₂OCH₂CH₂CH₂), 71.3 (2×OSO₃CH₂CH₂), 71.4 (2×OSO₃CH₂CH₂OCH₂), 72.7 (2×OCH₂CH₂CH₂); ESI MS (neg ion mode): calc for (M+Na) m/z C₃₃H₆₆O₁₂S₂Na 741.39. found 741.5; calc for (M)/2, 359.20. found 359.3; calc for M+H, 719.40. found 719.2; calc for 2M+Na, 1505.77. found 1506.3. Anal. Calc. for C₃₃H₆₄O₁₂S₂Na₂: C, 51.81; H, 8.70. Found: C, 52.04; H, 8.86.

### Example 23D

### Sodium 5,5-bis(tetradecyloxymethyl)-3,7-dioxa-1,9-nonanediol disulfate (23d)

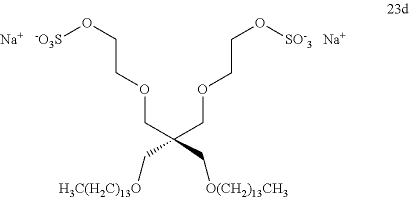

To an ice-bath cooled solution of 2,2-bis(tetradecyloxymethyl)-1,3-propanediol 2d (Example 2D) (2.5 g, 0.0047 mol) in THF (75 mL) is added a hexanes-washed 60% oil dispersion of NaH (1.0 g, 0.0236 mol, 5.0 eq). The cooled reaction mixture is stirred for 30 min then a solution of ethylene sulfate (prepared by the method of Baker, W.; Field, F. B. J. Chem. Soc. 1932, 86-91 and/or Brimacombe, J. S.; Foster, A. B.; Hancock, E. B.; Overend, W. G.; Stacey, M. J. Chem. Soc. 1960, 201-211) (1.29 g, 0.0104 mol, 2.2 eq) in THF (20 mL) is added dropwise. When the addition is complete, the reaction mixture is heated to 40° C., then stirred for 24 h. After 24 h, more NaH (0.19 g 1.0 eq) and ethylene sulfate (0.645 g, 0.005 mol, 1.1 eq) are added at 0° C., then the reaction mixture is allowed to warm to 40° C. and then stirred for another 24 h. MeOH is added carefully dropwise until foaming ceases, then 10% HCl is added until the reaction mixture is neutral (pH paper). Concentration yields a light yellow solid that is purified using flash column chromatography eluting with ethyl acetate-methanol mixtures changing from 95:05 to 80:20 to afford the title compound (23d) as a colorless powder, that is precipitated by addition of water and then recrystallized from ethyl acetate methanol to yield a colorless crystalline product: yield: 2.34 g, 60.3%; mp 145° C., became transparent, 160° C. melted; ¹H NMR (500.13 MHz, MeOD) δ 0.90 (t, J=7.5 Hz, 6H, 2×CH₃), 1.29 (br m, 40H, alkyl protons), 1.54 (quintet, J=6.5 Hz, 4H, 2×OCH₂CH₂), 3.35 (s, 4H, 2×CH₂CH₂CH₂OCH₂C), 3.39 (t, J=6.5 Hz, 4H, 2×OCH₂CH₂CH₂), 3.46 (s, 4H, O₃SOCH₂CH₂OCH₂C), 3.64 (AA′ part of AA′XX′ pattern, J_{AX}+J_{A′X}=10.0 Hz, 4H, 2×⁻O₃SOCH₂CH₂), 4.09 (XX′ part of AA′XX′ pattern, J_{AX}+J_{A′X}=10.0 Hz, 4H, 2×⁻O₃SOCH₂CH₂); ¹³C NMR (125.77 MHz, MeOD) δ 14.4 (2×CH₃), 23.7 (2×CH₂CH₃), 27.4 (2×OCH₂CH₂CH₂), 30.5, 30.6, 30.8, 30.8, 30.8 (2×OCH₂CH₂ and 12 alkyl chain carbons), 33.1 (2×CH₂CH₂CH₃), 46.8 (q C), 68.1 (2×OSO₃CH₂), 70.5 (CH₂OCH₂CH₂CH₂), 71.2 (2×OSO₃CH₂CH₂), 71.3 (2×OSO₃CH₂CH₂OCH₂), 72.5 (2×OCH₂CH₂CH₂); HRMS calc for (M−Na) C₃₇H₇₄O₁₂S₂Na 797.452. found 797.442.

### Example 24A

### Disodium 6,6-bis(octyloxymethyl)-4,8-dioxa-1,1′-undecanedisulfonate

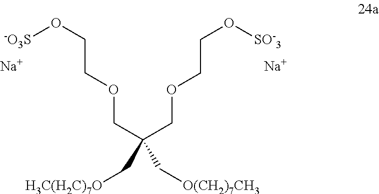

1,3-Propanesultone (4.1 mL, 10 eq.) in THF (5 mL) is added to a stirred solution of 2,2-bis(octyloxymethyl)-1,3-propanediol (Compound 2a, Example 2A) (1.7 g, 4.7 mmol) in THF (5 mL) under an Ar atmosphere and then a suspension of hexane-washed sodium hydride (60% oil dispersion, 0.43 g, 10.6 mmol, 2.25 eq) in THF (3 mL) is added dropwise over a 20-30 min. period. The resulting mixture is stirred for 18 h at 40° C. Another addition of a suspension of sodium hydride (60% oil dispersion, 0.21 g, 5.34 mmol, 1.13 eq) in THF (3 mL) is made and the reaction mixture is stirred for an additional 6 h at 40° C., then for 24 h at rt. Methanol is added dropwise to the ice-bath cooled reaction mixture until foaming ceases. 10% HCl is added until the reaction mixture is neutral (pH paper). Concentration gave a white solid that is purified using flash column chromatography initially eluting with an ethyl acetate/ethanol mixture (80:20 v:v) and then eluting with ethanol/ethyl acetate (80:20 v:v) to afford the title compound as a white solid that is precipitated out from ethanol/water and then recrystallized from ethyl acetate/methanol to yield colorless granules: yield 2.31 g, 76%; mp 185° C. becomes transparent, 220-235° C., decomposes; R_{F} 0.34 (butanol water methanol: 10 2.5 1.5); ¹H NMR (500.13 MHz, methanol-d₄) δ 0.90 (t, J=6.9 Hz, 6H, 2×CH₃), 1.31 (br m, 20H, alkyl protons), 1.52 (pentet, J=6.7 Hz, 4H, 2×OCH₂CH₂CH₂CH₂), 2.02 (m, 4H, 2×⁻O₃SCH₂CH₂), 2.87 (m, 4H, 2×⁻O₃SCH₂), 3.36 (s, 4H, 2×CH₂CH₂CH₂CH₂OCH₂C), 3.377 (t, 4H, J=6.3 Hz, 2×OCH₂CH₂CH₂), 3.383 (s, 4H, 2×⁻O₃SCH₂CH₂CH₂OCH₂C), 3.48 (t, 4H, J=6.1 Hz, 2×⁻O₃SCH₂CH₂CH₂); ¹³C NMR (125.77 MHz, methanol-d₄) δ 14.4 (2×CH₃), 23.7 (2×CH₂CH₃), 23.7 (2×⁻O₃SCH₂CH₂), 26.7 (2×OCH₂CH₂CH₂), 27.4, 30.47, 30.54, 30.74 (2×OCH₂CH₂ and alkyl chain carbons), 33.0 (2×CH₂CH₂CH₃), 46.7 (q C), 50.0 (2×⁻O₃SCH₂), 70.7 (2×CH₂OCH₂CH₂CH₂CH₂), 70.9 (2×⁻O₃SCH₂CH₂CH₂OCH₂), 71.2 (2×⁻O₃SCH₂CH₂CH₂O), 72.5 (OCH₂CH₂CH₂CH₂); HR ESI MS m/z calc for C₂₇H₅₄O₁₀S₂Na (M−Na) 625.3051. found 625.3082.

### Example 24B

### Disodium 6,6-bis(decyloxymethyl)-4,8-dioxa-1,1′-undecanedisulfonate

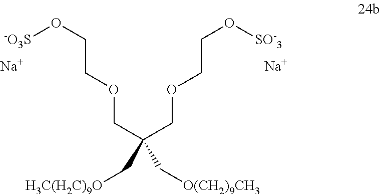

Compound 24b is made following the procedure of Example 24A except that workup of the neutralized reaction mixture is changed. 2,2-Bis(decyloxymethyl)-1,3-propanediol (Compound 2b, Example 2B) (1.72 g, 4.13 mmol) in THF (5 mL) is reacted with 1,3 propanesultone (4.0 mL, 10 eq) under Ar using two additions of suspensions of hexane-washed 60% oil dispersions of sodium hydride (0.37 g, 9.3 mmol, 2.25 eq) in THF (3 mL) and (0.186 g, 5.17 mmol, 1.13 eq) in THF (3 mL). The colorless solid resulting from concentration of the neutralized reaction mixture is washed repeatedly with ethyl acetate until NMR indicated that all sultone has been removed. The product is then extracted with hot ethanol. The hot ethanol extract is concentrated then the residue is taken up in boiling ethanol containing a few drops of water. Cooling results in a precipitate that is crystallized from ethyl acetate/methanol to give colorless granules of the title compound: yield 2.4 g, 83%; mp 187° C., becomes transparent, 215-230° C. decomposes; R_{F} 0.38 (butanol water methanol: 10 2.5 1.5); ¹H NMR (500.13 MHz, methanol-d₄) δ 0.89 (t, J=6.9 Hz, 6H, 2×CH₃), 1.34 (br m, 28H, alkyl protons), 1.54 (pentet, J=6.5 Hz, 4H, 2×OCH₂CH₂CH₂CH₂); 2.02 (m, 4H, 2×⁻O₃SCH₂CH₂), 2.89 (m, 4H, 2×⁻O₃SCH₂), 3.36 (s, 4H, 2×CH₂CH₂CH₂CH₂OCH₂C), 3.377 (t, 4H, J=6.3 Hz, 2×OCH₂CH₂CH₂), 3.382 (s, 4H, 2×⁻O₃SCH₂CH₂CH₂OCH₂C), 3.48 (t, 4H, J=6.1 Hz, 2×⁻O₃SCH₂CH₂CH₂); ¹H NMR (500.13 MHz, DMSO-d₆) δ 0.86 (t, J=6.8 Hz, 6H, 2×CH₃), 1.24 (br m, 28H, alkyl protons), 1.46 (pentet, J=6.6 Hz, 4H, 2×OCH₂CH₂CH₂CH₂), 1.77 (m, 4H, 2×⁻O₃SCH₂CH₂), 2.44 (m, 4H, 2×⁻O₃SCH₂), 3.255, 3.257 (2s, 8H, 2×CH₂CH₂CH₂CH₂OCH₂C, 2×⁻O₃SCH₂CH₂CH₂OCH₂C), 3.31 (t, 4H, J=6.4 Hz, 2×⁻O₃SCH₂CH₂CH₂), 3.36 (t, 4H, J=6.3 Hz, 2×⁻O₃SCH₂CH₂CH₂); ¹³C NMR (125.77 MHz, methanol-d₄) δ 14.4 (2×CH₃), 23.7 (2×CH₂CH₃), 23.7 (2×⁻O₃SCH₂CH₂), 26.7 (2×OCH₂CH₂CH₂), 27.4, 30.47, 30.58, 30.72, 30.81 (2×OCH₂CH₂ and alkyl chain carbons), 33.1 (2×CH₂CH₂CH₃), 46.7 (q C), 50.0 (2×⁻O₃SCH₂), 70.7 (2×CH₂O(CH₂)₉), 70.9 (2×⁻O₃SCH₂CH₂CH₂OCH₂), 71.2 (2×⁻O₃SCH₂CH₂CH₂O), 72.5 (OCH₂(CH₂)₈); HR ESI MS m/z calcd for C₃₁H₆₂NaO₁₀S₂ (M−Na) 681.3677. Found 681:3706.

### Example 24C

### Disodium 6,6-bis(dodecyloxymethyl)-4,8-dioxa-1,1′-undecanedisulfonate

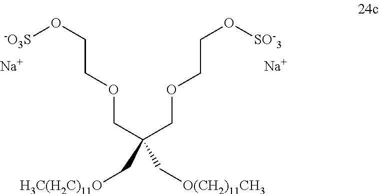

Compound 24c is made following the procedure of Example 24B. 2,2-Bis(dodecyloxymethyl)-1,3-propanediol (Compound 2c, Example 2C) (1.32 g, 2.79 mmol) in THF (5 mL) is reacted with 1,3 propanesultone (2.5 mL, 28 mmol, 10 eq) in THF (5 mL) under Ar using two additions of suspensions of hexane-washed 60% oil dispersion of sodium hydride (0.25 g, 6.3 mmol, 2.25 eq) in THF (3 mL) and 1.13 eq in THF (3 mL). The colorless solid resulting from concentration of the neutralized reaction mixture is washed repeatedly with ethyl acetate until NMR indicates that all sultone had been removed. The product is then extracted with hot ethanol. The hot ethanol extract is concentrated then the residue is taken up in boiling ethanol containing a few drops of water. Cooling results in a precipitate that is crystallized from ethyl acetate/methanol to give colorless granules of the title compound: yield 1.80 g, 85%; mp 180° C., becomes transparent, 215-240° C., decomposes; R_{F} 0.41 (butanol water methanol: 10 2.5 1.5); ¹H NMR (500.13 MHz, methanol-d₄) δ 0.89 (t, J=6.9 Hz, 6H, 2×CH₃), 1.29-1.35 (br s, 36H, 18×CH₂), 1.56 (pentet, J=6.6 Hz, 4H, 2×OCH₂CH₂CH₂CH₂); 2.02 (m, 4H, 2×⁻O₃SCH₂CH₂), 2.87 (m, 4H, 2×⁻O₃SCH₂), 3.36 (s, 4H, 2×(CH₂)₁₁OCH₂C), 3.377 (t, 4H, J=6.3 Hz, 2×OCH₂(CH₂)₁₀), 3.382 (s, 4H, 2×⁻O₃SCH₂CH₂CH₂OCH₂C), 3.48 (t, 4H, J=6.1 Hz, 2×⁻O₃SCH₂CH₂CH₂); ¹³C NMR (125.77 MHz, MeOD) δ 14.5 (2×CH₃), 23.7 (2×CH₂CH₃), 23.7 (2×⁻O₃SCH₂CH₂), 26.7 (2×OCH₂CH₂CH₂), 27.4, 30.49, 30.59, 30.65, 30.78, 30.82 (2×OCH₂CH₂ and alkyl chain carbons), 33.1 (2×CH₂CH₂CH₃), 46.7 (q C), 50.0 (2×⁻O₃SCH₂), 70.7 (2×CH₂O(CH₂)₁₁), 70.9 (2×⁻O₃SCH₂CH₂CH₂OCH₂), 71.2 (2×⁻O₃S(CH₂)₂CH₂O), 72.5 (OCH₂(CH₂)₁₀); HR ESI MS m/z calc for C₃₅H₇₀NaO₁₀S₂ (M−Na) 737.4303. found 737.4256.

### Example 24D

### Disodium 4,8-dioxa-6,6-bis(tetradecyloxymethyl)-1,11-undecanedisulfonate

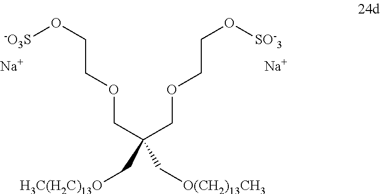

Compound 24d is made following the procedure of Example 24B from 1,3-propanesultone (2 mL, 21 mmol, 10 eq) in THF (5 mL), compound 2d (Example 2D) (1.13 g, 2.14 mmol) in dry THF (5 mL) using two identical additions of suspensions of 60% oil dispersions of sodium hydride (0.2 g, 4.3 mmol, 2.25 eq), each followed by stirring for 12 h at 40° C., then a third addition (0.1 g, 2.4 mmol, 1.13 eq) followed by stirring at 35° C. for 24 h and then for 12 h at rt. Normal work up gives a white solid that is purified by column chromatography as for Example 24A followed by crystallization from ethyl acetate/methanol to give an amorphous colorless solid: yield 1.43 g, 82%; mp 175-180° C., becomes transparent, 210-245° C. decomposes; R_{F} 0.44 (butanol water methanol: 10 2.5 1.5); ¹H NMR (500.13 MHz, methanol-d₄) δ 0.89 (t, J=6.8 Hz, 6H, 2×CH₃), 1.28-1.31 (br s, 44H, 22×CH₂), 1.54 pentet, J=6.5 Hz, 4H, 2×OCH₂CH₂(CH₂)₁₁); 2.05 (m, 4H, 2×⁻O₃SCH₂CH₂), 2.91 (m, 4H, 2×⁻O₃SCH₂), 3.36 (s, 4H, 2×(CH₂)₁₃OCH₂C), 3.377 (t, 4H, J=6.3 Hz, 2×OCH₂(CH₂)₁₃), 3.382 (s, 4H, 2×⁻O₃S(CH₂)₃OCH₂C), 3.48 (t, 4H, J=6.1 Hz, 2×⁻O₃S(CH₂)₂CH₂O); ¹³C NMR (125.77 MHz, D₂O) δ 13.9 (2×CH₃), 22.8 (2×CH₂CH₃), 24.7 (2×⁻O₃SCH₂CH₂), 26.4 (2×OCH₂CH₂CH₂), 29.7-30.1 (2×OCH₂CH₂ and alkyl chain carbons), 32.1 (2×CH₂CH₂CH₃), 45.3 (q C), 48.3 (2×⁻O₃SCH₂), 69.2 (2×CH₂O(CH₂)₁₃), 69.6 (2×⁻O₃S(CH₂)₃OCH₂), 70.1 (2×⁻O₃S(CH₂)₂CH₂O), 71.5 (OCH₂(CH₂)₁₂); HR ESI MS m/z calc for C₃₉H₇₈NaO₁₀S₂ (M−Na) 793.4929. found 793.4929.

### Example 25

### Physicochemical Properties of Gemini Surfactants

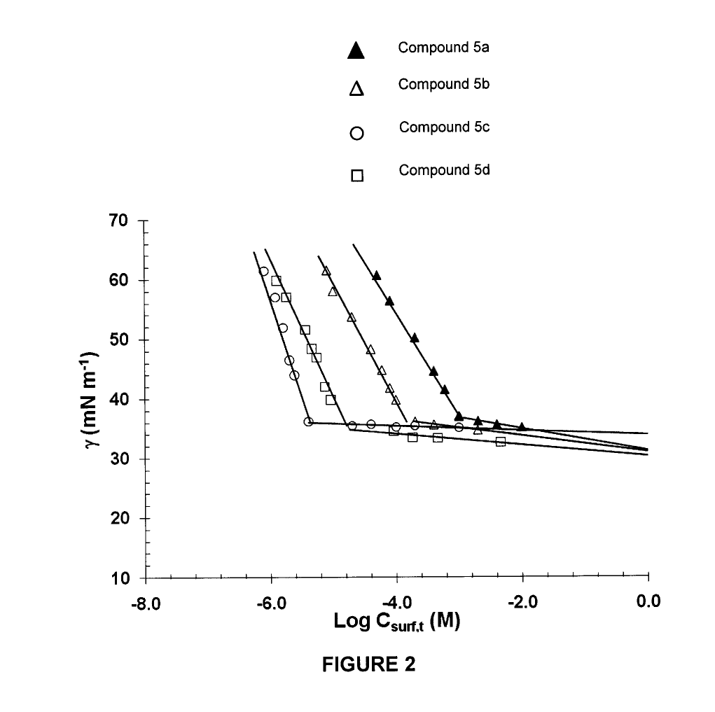

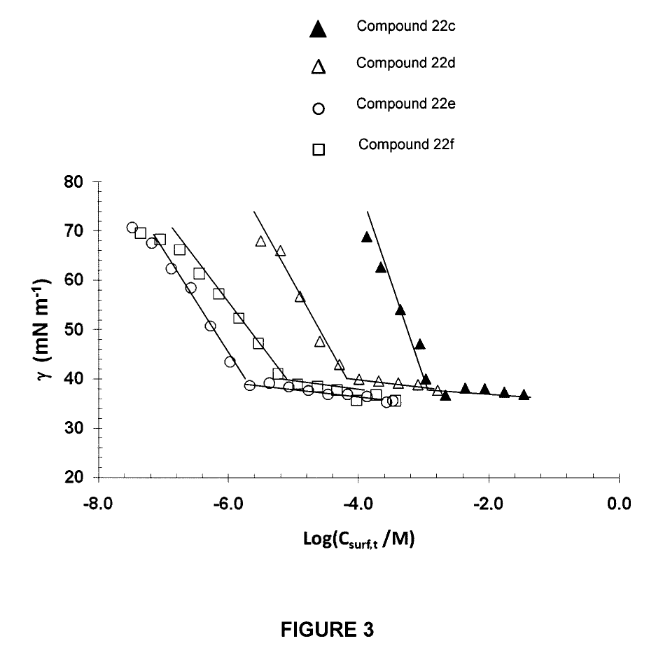

Equilibrium surface tension measurements (γ values) are performed using the Wilhelmy plate technique. Measurements of surface tension are performed with either a KRUSS K8 manual or K10 digital tensiometer; the accuracy is ±0.1 mN·m⁻¹. All measurements are done in a jacketed beaker at 20.0° C., using either a Haake or Neslab refrigerated bath (±0.2° C.). In general, 10-15 concentration points for each surfactant/water system are obtained. The results from either duplicate or triplicate trials are averaged to obtain the surface tension (γ) versus log of the total surfactant concentration (C_{surf,t}) profiles. FIG. 2 shows a plot of surface tension (mN·m⁻¹) versus log₁₀ of the total surfactant concentration (molar) for compounds 5a-5d (Examples 5A-5D) and FIG. 3 is a plot of surface tension (mN·m⁻¹) versus log₁₀ of the total surfactant concentration (molar) for compounds 22c-22f (Examples 22C-22F).

Critical micelle concentration (CMC) is determined by a linear regression analysis of both the pre and post-micellar lines of each surface tension plot to identify the intersection point. (Dreger, E. E.; Keim, G. I.; Miles, G. D.; Shedlovsky, L.; Ross, J. Ind. Eng. Chem. 1944, 36, 610-617; Boucher, E. A.; Grinchuk, T. M.; Zettlemo, A. C. J. Colloid. Interface Sci. 1967, 23, 600-603). The surface excess concentration (F) of a surfactant can be approximated as the actual surface concentration without introducing considerable error (Song, L. D.; Rosen, M. J. Langmuir 1996, 12, 1149-1153). The surfactant concentration at the interface is calculated from the surface tension data using the Gibbs equation:

$$Γ = - \frac{1}{nRT} {( \frac{\partial γ}{\partial ln ( C )} )}_{T}$$

The differential term in the Gibbs equation is obtained from the slope of a plot of γ (surface tension) versus the natural logarithm of surfactant concentration at constant temperature. For dimeric surfactants, n=3. When γ is in mN·m⁻¹ and R=8.314 J·mol⁻¹·K⁻¹, F will have units of mol/1000 m².

The area of one monomer (Aₘᵢₙ) at the interface can be determined from the surface excess using the following equation (Rosen, M. J. Chemtech 1993, 23, 30-33; Boucher, E. A.; Grinchuk, T. M.; Zettlemo, A. C. J. Colloid. Interface Sci. 1967, 23, 600-603; Song, L. D.; Rosen, M. J. Langmuir 1996, 12, 1149-1153; Rosen, M. J.; Song, L. D. J. Colloid. Interface Sci. 1996, 179, 261-268):

$${A}_{min} = \frac{1}{{N}_{Avo} Γ}$$

where N_{Avo}=Avogadro's number and Γ is in mol/m². The CMC values are substituted into the linear regression equation of the pre-micellar line to determine the surface tension at the CMC. The surfactant concentration which lowers the surface tension by 20 mN/m (C₂₀) is determined by substituting 52 mN·m⁻¹ into the linear regression equation for the pre-micellar line and solving for the appropriate concentration. The CMC values for compounds 5a to 5d (Examples 5A to 5D) are given in Table 1.

TABLE 1
CMC and surface tension derived quantities
for compounds 5a-5d (Examples 5A-5D).

| | Chain | CMC | | γ_{CMC} | Aₘᵢₙ |
|---|---|---|---|---|---|
| Compound | Length | (10⁻³ mol L⁻¹) | pC₂₀ | (mN/M) | (nm²) |
| 5a | 8 | 1.19 | 3.59 | 31.9 | 1.53 |
| 5b | 10 | 0.165 | 4.70 | 32.2 | 1.61 |
| 5c | 12 | 0.00159 | 5.92 | 33.1 | 0.355 |
| 5d | 14 | 0.000565 | 6.41 | 32.6 | 0.302 |

From the results in Table 1, it can be seen that, in at least one embodiment, the CMC values of the compounds of formula I decrease as the alkyl chain length increases. A corresponding trend in C₂₀ values is seen. The area/monomer at the interface is relatively large, consistent with the presence of the two cationic head groups at the interface.

A comparison of CMC values for some surfactants of the present invention with other conventional and known gemini surfactants is given in Table 2.

TABLE 2
Comparison of CMC and surface tension quantities for some compounds of formula I and common conventional and dimeric surfactants

| | | CMC | | γ_{CMC} | Aₘᵢₙ |
|---|---|---|---|---|---|
| Surfactant | | (mmol · L⁻¹) | pC₂₀ | (mN · m⁻¹) | (nm²) |
| N-Dodecyl-N,N,N-trimethylammonium bromide (12-TAB)¹ | | 15.5 | 2.10 | 39 | 71 |
| Sodium dodecyl sulfate (SDS)¹ | | 8.3 | 2.50 | 39 | 76 |
| Compound 5c (Example 5C) | | 0.0159 | 5.92 | 33.1 | 0.36 |
| Compound 10c (Example 10C) | | 0.0533 | 5.43 | 31.5 | 1.00 |
| Compound 21c (Example 21C) | | 0.0281 | 5.20 | 33.0 | 0.71 |
| Compound 5n (Example 5N) | | 0.0163 | 5.44 | 34.8 | 0.80 |
| Compound 22e (Example 22E) | | 0.0255 | 5.77 | 36.1 | 2.10 |
| N,N′-didodecyl-N,N,N′,N′-tetramethyl-1,4-butanediammonium bromide (12-4-12)² | | 1.00 | 3.40 | 39.8 | 1.16 |
| N,N′-didodecyl-N,N,N′,N′-tetramethyl-1,6-hexanediammonium bromide (12-6-12)² | | 1.12 | 3.30 | 42.5 | 1.43 |
| 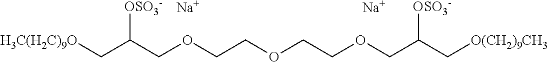 | ¹ | 0.060 | 5.0 | 36.0 | — |

¹Menger, F. M.; Keiper, J. S. Gemini Surfactants. Angewandte Chemie International Edition 2000, 39, 1906-1920.
²Alami, E.; Levy, H.; Zana, R. Alkanediyl-α,ω-Bis(Dimethylalkylammonium Bromide) Surfactants. 2. Structure of the Lyotropic Mesophases in the Presence of Water. Langmuir 1993, 9, 940-949.

It can be seen from Table 2 that, in at least one embodiment, the CMC values of the compounds of formula I are lower than that of single-headed, single-tailed and two-headed surfactants of various types. Furthermore, for at least one cationic series of the compounds of formula I, the CMC value increases with an increase in the number of CH₂ groups in the methylene spacer, reaching a maximum with four methylene chains and decreasing thereafter. In addition, in at least one embodiment, CMC values appear to be the smallest when the spacer group is a short, slightly hydrophilic chain or a flexible hydrophobic chain.

In the following Tables 3 to 6, physicochemical properties of the surfactants of the present invention are compared against those of known monomeric and dimeric surfactants. Comparative monomeric surfactants, denoted n-TAB, where n is the number of carbon atoms in the CₙH₂ₙ₊₁ alkyl chain, have the following chemical structure (Menger, F. M.; Keiper, J. S. Gemini Surfactants. Angewandte Chemie International Edition 2000, 39, 1906-1920):

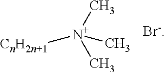

Comparative dimeric surfactants, denoted n-5-n, where n is the number of carbon atoms in the CₙH₂ₙ₊₁ alkyl chain, have the following chemical structure (Alami, E.; Levy, H.; Zana, R. Alkanediyl-α,ω-Bis(Dimethylalkylammonium Bromide) Surfactants. 2. Structure of the Lyotropic Mesophases in the Presence of Water. Langmuir 1993, 9, 940-949):

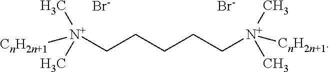

The CMC results of the compounds of formula (I) as well as comparative monomeric and dimeric surfactants are compared in the following table.

TABLE 3
CMC comparisons for the compounds of
formula I with comparator compounds

| | Comparative | Comparative | |
|---|---|---|---|
| | monomeric | dimeric | Compounds of |
| Chain | surfactants | surfactants | formula I |
| Length | (n-TAB) | (n-5-n) | (Examples 5b-5d) |
| (n) | (M) | (M) | (M) |
| 10 | 6.2 × 10⁻² | 1.7 × 10⁻² | 1.7 × 10⁻⁴ |
| 12 | 1.4 × 10⁻² | 1.2 × 10⁻³ | 1.6 × 10⁻⁶ |
| 14 | 3.6 × 10⁻³ | 1.1 × 10⁻⁴ | 5.7 × 10⁻⁷ |

The n-5-n dimeric surfactants are known to have CMC values that are commonly lower than those of their analogous monomeric surfactants (Rosen, M. J.; Tracy, D. J. J. Surfact. Det. 1998, 1, 547-554); this is observed here. The results in Table 3 indicate that in at least one embodiment, compounds of formula I can form micelles at a lower concentration than either the n-5-n or n-TAB compounds. This result indicates that in at least one embodiment, surfactants of the present invention could be used in concentrations appreciably smaller than those of conventional dimeric surfactants while continuing to provide their maximum surface tension lowering effect.

TABLE 4
Comparison of pC₂₀ for the compounds of
formula I with comparator compounds.

| | Comparative | Comparative | |
|---|---|---|---|
| Chain | monomeric | dimeric | Compounds of |
| Length | surfactants | surfactants | formula I |
| (n) | (n-TAB) | (n-5-n) | (Examples 5b-5c) |
| 10 | 2.1 | 4.0 | 4.7 |
| 12 | 2.8 | 4.9 | 5.9 |

From these results, it can be seen that the C₂₀ value decreases going from the monomeric n-TAB surfactants to the dimeric n-5-n surfactants; the results for surfactants of the present invention show a further decrease. These results support the low concentration of at least one embodiment of the compounds of formula I necessary to have a large effect on the surface tension.

TABLE 5
Comparison of γ_{CMC} values for the compounds
of formula I with comparator compounds.

| | Comparative | Comparative | |
|---|---|---|---|
| Chain | monomeric | dimeric | Compounds of |
| Length | surfactants | surfactants | formula I |
| (n) | (n-TAB) | (n-5-n) | (Examples 5c-5d) |
| 12 | 39 | 40 | 32 |
| 14 | 38 | 39 | 33 |

The results in Table 5 show that, in at least one embodiment, the γ_{CMC} values for the compounds of formula I are lower than those of the comparator compounds.

TABLE 6
Comparison of Aₘᵢₙ at the air water interface for the
compounds of formula I with comparator compounds

| | Comparative | Comparative | |
|---|---|---|---|
| Chain | monomeric | dimeric | Compounds of |
| Length | surfactants | surfactants | formula I |
| (n) | (n-TAB) | (n-5-n) | (Examples 5b-5d) |
| 10 | | | 1.24 |
| 12 | 0.49 | 1.70 | 0.34 |
| 14 | 0.61 | 1.56 | 0.29 |

The results shown in Table 6 show that, in at least one embodiment, the Aₘᵢₙ for the compounds of formula I can be slightly lower than that for the monomeric surfactants.

One of the main reasons for the extensive use of surfactants in commercial and industrial applications is to reduce the surface tension of water or the interfacial tension of a hydrocarbon/water interface, for example. Two key parameters related to the equilibrium surface tension lowering ability of surfactants are the C₂₀ value (the concentration of surfactant required to lower the surface tension of the solvent by 20 dynes/cm) and the surface tension at the CMC (the γ_{CMC} value). It is clear from the data in Tables 4 and 5 that for at least one embodiment of the surfactants of the present invention listed, the C₂₀ values are lower than those of comparable monomeric and dimeric surfactants. Furthermore, the γ_{CMC} values of at least one embodiment of the surfactants of the present invention are comparable to or lower than those of comparator compounds.

Because of these properties, it is envisioned that in at least one embodiment, the compounds of formula I can be more cost-effective than are conventional surfactants. For example, in at least one embodiment, the compounds of formula I can act to reduce surface tension and/or can form micelles at concentrations lower than those required of conventional surfactants. In addition, in at least one embodiment, the compounds of formula I can have C₂₀ values (the concentration of surfactant required to lower the surface tension by 20 dynes/cm) lower than those of conventional surfactants. Thus, in at least one embodiment, the amount of the compounds of formula I required can be substantially reduced compared to the amount of conventional surfactants required for the same application. Furthermore, in at least one embodiment, the compounds of formula I can be easy to dispose of, potentially providing further cost savings.

The previous detailed description is provided to enable any person skilled in the art to make or use the present invention. Various modifications to those embodiments will be readily apparent to those skilled in the art, and the generic principles defined herein may be applied to other embodiments without departing from the spirit or scope of the invention described herein. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims.

1. A compound of formula IA 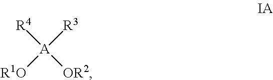
wherein A is a core derived from an organic polyhydroxy compound;
R¹ and R² are each independently a hydrophobic group; and
R³ and R⁴ are each independently a surfactant headgroup.

2. The compound according to claim 1 wherein A is a core derived from pentaerythritol, such that the compound is a compound of formula I 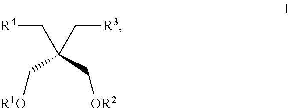
wherein R¹, R², R³ and R⁴ are defined as in claim 1.

3. The compound according to claim 1 wherein R¹ is identical to R².

4. The compound according to claim 1 wherein R¹ and R² are each independently a hydrophobic group selected from (C₁₋₂₄)alkyl, aryl(C₁₋₂₄)alkyl and (C₁₋₂₀)hydroxyalkylpolyoxyalkylene; wherein the aryl(C₁₋₂₄)alkyl is optionally substituted with from one to three (C₁₋₂₄)alkyl groups; and wherein the (C₁₋₂₄)alkyl is optionally substituted with hydroxyl, (C₁₋₂₄)alkoxy, (C₁₋₂₄)alkyl-C(═O)NH—, or (C₁₋₂₄)alkyl-NHC(═O)—.

5. The compound according to claim 1 wherein R³ is identical to R⁴.

6. The compound according to claim 1 wherein R³ and R⁴ are each independently a surfactant headgroup selected from —OH, —SO₃⁻, —(C₁₋₆)alkyl-SO₃⁻, —O(C₁₋₆)alkyl-SO₃⁻, —OSO₃⁻, —(C₁₋₆)alkyl-OSO₃⁻, —O(C₂₋₆)alkyl-OSO₃⁻, —COO⁻, —(C₁₋₆)alkyl-COO⁻, —O(C₁₋₆)alkyl-COO⁻, —PO₃²⁻, —(C₁₋₆) alkyl-PO₃²⁻, —O(C₁₋₆)alkyl-PO₃²⁻, —PO₃H⁻, —(C₁₋₆) alkyl-PO₃H⁻, —O(C₁₋₆)alkyl-PO₃H⁻, —OPO₃²⁻, —(C₁₋₆) alkyl-OPO₃²⁻, —O(C₂₋₆) alkyl-OPO₃²⁻, —OPO₃H⁻, —(C₁₋₆) alkyl-OPO₃H⁻, —O(C₂₋₆) alkyl-OPO₃H⁻, —N(R⁵)(R⁶)(R⁷)⁺, —(C₁₋₆) alkyl-N(R⁵)(R⁶)(R⁷)⁺, and —O(C₂₋₆) alkyl-N(R⁵)(R⁶)(R⁷)⁺;
wherein R⁵, R⁶ and R⁷ are each independently in each instance H, —(C₁₋₆)alkyl, —(C₂₋₆)alkyl-OH, —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻, —(C₁₋₆)alkyl-COO⁻, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

7. The compound according to claim 6 wherein at least one of R³ and R⁴ is an anionic surfactant headgroup selected from —SO₃⁻, —(C₁₋₆)alkyl-SO₃⁻, —O(C₁₋₆)alkyl-SO₃⁻, —OSO₃⁻, —(C₁₋₆)alkyl-OSO₃⁻, —O(C₂₋₆)alkyl-OSO₃⁻, —COO⁻, —(C₁₋₆)alkyl-COO⁻, —O(C₁₋₆)alkyl-COO⁻, —PO₃²⁻, —(C₁₋₆)alkyl-PO₃²⁻, —O(C₁₋₆)alkyl-PO₃²⁻, —PO₃H⁻, —(C₁₋₆)alkyl-PO₃H⁻, —O(C₁₋₆)alkyl-PO₃H⁻, —OPO₃²⁻, —(C₁₋₆)alkyl-OPO₃²⁻, —O(C₂₋₆)alkyl-OPO₃²⁻, —OPO₃H⁻, —(C₁₋₆) alkyl-OPO₃H⁻ and —O(C₂₋₆) alkyl-OPO₃H⁻.

8. The compound according to claim 6 wherein at least one of R³ and R⁴ is a cationic surfactant headgroup selected from —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance H, —(C₁₋₆)alkyl, —(C₂₋₆)alkyl-OH, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

9. The compound according to claim 6 wherein at least one of R³ and R⁴ is a zwitterionic surfactant headgroup selected from —N(R⁵)(R⁶)(R⁷)⁺, —(C₁₋₆)alkyl-N(R⁵)(R⁶)(R⁷)⁺, and —O(C₂₋₆) alkyl-N(R⁵)(R⁶)(R⁷)⁺;
wherein one of R⁵, R⁶ and R⁷ is —(C₁₋₆)alkyl-SO₃⁻, —(C₂₋₆)alkyl-OSO₃⁻, —(C₁₋₆) alkyl-PO₃H⁻, —(C₂₋₆) alkyl-OPO₃H⁻ or —(C₁₋₆)alkyl-COO⁻ and the remaining two of R⁵, R⁶ and R⁷ are each independently in each instance H, —(C₁₋₆)alkyl, —(C₂₋₆)alkyl-OH, or the remaining two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

10. A compound of formula I 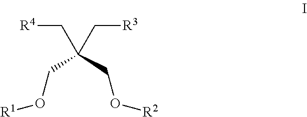
wherein R⁴ and R² are each independently a hydrophobic group selected from (C₁₋₂₄)alkyl, aryl(C₁₋₂₄)alkyl and (C₁₋₂₀)hydroxyalkylpolyoxyalkylene; wherein the aryl(C₁₋₂₀)alkyl is optionally substituted with from one to three (C₁₋₂₄) alkyl groups; and wherein the (C₁₋₂₄)alkyl is optionally substituted with hydroxyl, (C₁₋₂₄)alkoxy, (C₁₋₂₄)alkyl-C(═O)NH—, or (C₁₋₂₄)alkyl-NHC(═O)—; and
R³ and R⁴ are each independently a surfactant headgroup selected from —OH, —SO₃⁻, —(C₁₋₆)alkyl-SO₃⁻, —O(C₁₋₆)alkyl-SO₃⁻, —OSO₃⁻, —(C₁₋₆)alkyl-OSO₃⁻, —O(C₂₋₆) alkyl-OSO₃⁻, —COO⁻, —(C₁₋₆)alkyl-COO⁻, —O(C₁₋₆) alkyl-COO⁻, —PO₃²⁻, —(C₁₋₆)alkyl-PO₃²⁻, —O(C₁₋₆)alkyl-PO₃²⁻, —PO₃H⁻, —(C₁₋₆)alkyl-PO₃H⁻, —O(C₁₋₆)alkyl-PO₃H⁻, —OPO₃²⁻, —(C₁₋₆)alkyl-OPO₃²⁻, —O(C₂₋₆)alkyl-OPO₃²⁻, —OP₃H⁻, —(C₁₋₆)alkyl-OPO₃H⁻, —O(C₂₋₆)alkyl-OPO₃H⁻, —N(R⁵)(R⁶)(R⁷)⁺, —(C₁₋₆)alkyl-N(R⁵)(R⁶)(R⁷)⁺, and —O(C₂₋₆)alkyl-N(R⁵)(R⁶)(R⁷)⁺;
wherein R⁵, R⁶ and R⁷ are each independently in each instance H, —(C₁₋₆)alkyl, —(C₂₋₆)alkyl-OH, —(C₁₋₆)alkyl-SO₃⁻, —(C₂₋₆)alkyl-OSO₃⁻, —(C₁₋₆)alkyl-PO₃H⁻, —(C₂₋₆)alkyl-OPO₃H⁻, —(C₁₋₆)alkyl-COO⁻, or at least two of R⁵, R⁶ and R⁷ are joined, together with the N atom to which they are attached, to form a heterocycle containing one N heteroatom and optionally from 1 to 3 further heteroatoms each independently selected from N, O and S, the heterocycle being optionally substituted with from one to three substituents each independently selected from (C₁₋₆)alkyl and aryl.

11. Use of a compound according to claim 1 as a surfactant.

12. A fluid for the production or recovery of petroleum from petroleum-bearing formations, the fluid comprising a compound according to claim 1, a base fluid and optionally at least one chemical additive.

13. A detergent composition comprising a compound according to claim 1 and at least one adjuvant, diluent or additive.

14. An emulsion composition comprising a compound according to claim 1, water, and at least one oil component.

15. Use of a compound according to claim 1 in the preparation of a fluid for the production or recovery of petroleum from petroleum-bearing formations.

16. Use of a compound according to claim 1 in the preparation of a detergent composition.

17. Use of a compound according to claim 1 in the preparation of an emulsion composition.

18. Use of a compound according to claim 1 as a scouring agent, a foaming agent, a defoamer, a demulsifying agent, a dispersant, a wetting agent, a dissolving agent, a lustering agent, a delustering agent, a softening agent, a water repellent, a flame repellent, an antistatic agent, or a flotation agent.
